SEQUENCE LISTING (26 sequences)

SEQ ID NO: 1 - DNA, 18 bp, Artificial Sequence
  PPAR Forward Primer
tgaccatggttgacaccg

SEQ ID NO: 2 - DNA, 20 bp, Artificial Sequence
  PPAR Reverse Primer
aagcatgaactccatagtgg

SEQ ID NO: 3 - DNA, 20 bp, Artificial Sequence
  C/EPB alpha Forward Primer
aaggccaagaagtcggtgga

SEQ ID NO: 4 - DNA, 20 bp, Artificial Sequence
  C/EBP alpha Reverse Primer
cagttcacggctcagctgtt

SEQ ID NO: 5 - DNA, 21 bp, Artificial Sequence
  GATA-1 Forward Primer
tgcaacacaccacccgatacc

SEQ ID NO: 6 - DNA, 21 bp, Artificial Sequence
  GATA-2 Reverse Primer
caatttgcacaacaggtgccc

SEQ ID NO: 7 - DNA, 22 bp, Artificial Sequence
  GATA-3 Forward Primer
tctcactctcgaggcagcatga

SEQ ID NO: 8 - DNA, 21 bp, Artificial Sequence
  GATA-3 Reverse Primer
ggtaccatctcgccgccacag

SEQ ID NO: 9 - DNA, 18 bp, Artificial Sequence
  Beta-Actin Forward Primer
cgctcgttgccaatagtg

SEQ ID NO: 10 - DNA, 20 bp, Artificial Sequence
  Beta-Actin Reverse Primer
gctgtgctatgttgctctag

SEQ ID NO: 11 - DNA, 24 bp, Artificial Sequence
  SREBP-1 Forward Primer
tatggagggcatgaaacccgaagt

SEQ ID NO: 12 - DNA, 24 bp, Artificial Sequence
  SREBP-1 Reverse Primer
ttgacctggctatcctcaaaggct

SEQ ID NO: 13 - DNA, 24 bp, Artificial Sequence
  PPAR gamma Forward Primer
tggaattagatgacagtgacttgg

SEQ ID NO: 14 - DNA, 21 bp, Artificial Sequence
  PPAR gamma Reverse Primer
ctctgtgacgatctgcctgag

SEQ ID NO: 15 - DNA, 24 bp, Artificial Sequence
  C/EBP alpha Forward Primer
agaagtcggtggacaagaacagca

SEQ ID NO: 16 - DNA, 24 bp, Artificial Sequence
  C/EBP alpha Reverse Primer
gcgttgtttggctttatctcggct

SEQ ID NO: 17 - DNA, 24 bp, Artificial Sequence
  LDLR Forward Primer
caacaatggtggctgttcccacat

SEQ ID NO: 18 - DNA, 24 bp, Artificial Sequence
  LDLR Reverse Primer
actcacacttgtagctgccttcca

SEQ ID NO: 19 - DNA, 24 bp, Artificial Sequence
  Adiponectin Forward Primer
agacctggccactttctcctcatt

SEQ ID NO: 20 - DNA, 24 bp, Artificial Sequence
  Adiponectin Reverse Primer
agaggaacaggagagcttgcaaca

SEQ ID NO: 21 - DNA, 24 bp, Artificial Sequence
  FAS Forward Primer
ggtgtggtgggtttggtgaattgt

SEQ ID NO: 22 - DNA, 24 bp, Artificial Sequence
  FAS Reverse Primer
tcacgaggtcatgctttagcacct

SEQ ID NO: 23 - DNA, 24 bp, Artificial Sequence
  Beta-Actin Forward Primer
tgtgatggtgggaatgggtcagaa

SEQ ID NO: 24 - DNA, 24 bp, Artificial Sequence
  Beta-Actin Reverse Primer
tgtggtgccagatcttctccatgt

SEQ ID NO: 25 - DNA, 20 bp, Artificial Sequence
  5-HTTLPR Allele Forward Primer
ggcgttgccgctctgaatgc

SEQ ID NO: 26 - DNA, 23 bp, Artificial Sequence
  5-HTTLPR Allele Reverse Primer
gagggactgagctggacaaccac

(19) United States
(12) Patent Application Publication
(10) Pub. No.: US 2011/0281852 A1
(43) Pub. Date: Nov. 17, 2011

(54) Pharmaceutical compositions containing berberine for treatment or prevention of weight gain and obesity associated with anti-psychotic drugs

(76) Inventors: Gareth Davies, Madison, SD (US); Yueshan Hu, Brookings, SD (US)

(21) Appl. No.: 13/069,375

(22) Filed: Mar. 22, 2011

Related U.S. Application Data
(60) Provisional application No. 61/340,728, filed on Mar. 22, 2010.

(51) Int. Cl. A61K 31/4375; A61K 31/519; A61P 3/04; A61K 31/551

(52) U.S. Cl. 514/220; 514/280; 514/259.41

(57) ABSTRACT

The compositions and methods disclosed herein are of use for the treatment and prevention of weight gain and obesity. In particular, the compositions and methods include treatment of weight gain and obesity with berberine and/or berberine analogs alone or in combination thereof to cause a reduction in an individual's weight or prevent weight gain or obesity. In certain embodiments, the weight gain and obesity are associated with administration of anti-psychotic drugs. In an alternative embodiment, the compositions and methods provide berberine or berberine analogs alone or for coadministration with an anti-psychotic agent. In an additional embodiment, the compositions and methods further include a natural product. The usefulness of the present invention is that berberine and berberine analogs do not have synergistic effects with other drugs and administration results in few side effects. Such characteristics of berberine or berberine analogs are a great improvement able to support the widespread use of the compositions and methods of the present invention as therapeutics for the treatment and prevention of weight gain and obesity.

### CROSS REFERENCE TO RELATED APPLICATION

This application claims benefit to U.S. Provisional Application No. 61/340,728 filed Mar. 22, 2010, under 35 U.S.C. 119(e), the contents of which are incorporated by reference herein in their entireties.

### BACKGROUND OF THE INVENTION

1. Field of the Invention

The present invention relates to a pharmaceutical composition for treating or preventing weight gain and obesity. The present invention also relates to a pharmaceutical composition for treating or preventing weight gain and obesity associated with administration of anti-psychotic drugs. More particularly, the present invention relates to a pharmaceutical composition comprising berberine (BBR) as an effective ingredient, where administration of the composition is associated with treatment or prevention of weight gain and obesity.

2. Background Information

There is irrefutable evidence that obesity is a threat to world health. The worldwide obesity epidemic has been strongly associated with the major risks of developing diseases such as cardiovascular disease, Type II diabetes, hypertension, hyperlipidemia, hypercholesterolemia, certain metabolic diseases and cancer. Obesity also leads to a large number of preventable deaths each year. Thus, the prevention and treatment of obesity is critical in maintaining worldwide health.

Unfortunately, there are not many effective pharmacologic treatments and many patients suffering from obesity are not willing or able to maintain a strict diet and exercise regime. In light of the extreme nature of this health problem and a general unwillingness by the majority of patients to diet and exercise, there is an urgent need for pharmacological agents to treat or prevent obesity.

### SUMMARY OF THE INVENTION

The present invention describes methods for the administration of berberine and/or its analogs to treat or prevent weight gain and obesity. The disclosure provides a therapeutic composition and method to treat individuals with obesity or at risk of developing it due to a predisposition by administering amounts of berberine and/or berberine analogs, either alone or in combination with known pharmacologic agents known to cause weight gain and obesity. The therapeutic composition and method to treat individuals may also include natural products in combination with the berberine and/or berberine analogs administered either alone or in combination with known pharmacologic agents. A predisposition may be due to a genetic cause and/or due to an individual taking a pharmacologic agent known to cause weight gain and obesity.

In accordance with the disclosure, a therapeutic amount of a berberine selected from the group consisting essentially of berberine, berberine analogs or combinations thereof is effective in treating individuals demonstrating obesity and/or at risk of developing it. The composition may further provide one or more natural products. In accordance with the disclosure, administration of a therapeutic amount of berberine, berberine analogs, or combination thereof is effective in reducing weight and/or preventing weight gain weight and the general detrimental characteristics associated with obesity. These detrimental characteristics include, but are not limited to, development of other conditions such as cardiovascular disease, Type II diabetes, hypertension, hyperlipidemia, hypercholesterolemia, certain metabolic diseases and cancer.

In accordance with the disclosure, a therapeutic amount of a berberine selected from the group consisting essentially of berberine, berberine analogs or combinations thereof is co-administered with pharmacologic agents known to cause obesity to treat individuals demonstrating obesity and/or at risk of developing it. The berberine may also include one or more natural products. An illustrative example of a natural product is amentoflavone. One skilled in the art is familiar with many types of such natural products. Illustrative examples of pharmacologic agents known to cause obesity include, but are not limited to, anti-psychotics, tranquilizers, antidepressants, anticonvulsants, and the like.

Berberine analogs useful in accordance with the instant disclosure are berberine salts, those compounds with very similar chemical classification as berberine, moieties and/or fragments, which demonstrate bioactivity similar or greater than berberine. Examples include berberine chloride, berberine phosphate, berberine sulphate, berberine bi-sulphate, berberine tannate, berberine hemisulphate, berberine citrate; or compounds with very similar chemical classification as berberine such as Sanguinarine, Coptisine, and Goldenseal.

### BRIEF DESCRIPTION OF THE DRAWINGS

The following figures form part of the present specification and are included to further demonstrate certain embodiments. These embodiments may be better understood by reference to one or more of these figures in combination with the detailed description of specific embodiments presented herein.

FIG. 1 illustrates the chemical structure of Berberine Chloride.

FIG. 2 illustrates the effects of Berberine on cell viabilities of 3T3-L1 cells during proliferation (A) and differentiation (B).

FIG. 3 illustrates berberine inhibiting 3T3-L1 adipocyte differentiation induced by differentiation medium (DM, includes differentiation medium 1 and 2) after 8 days. Cells were stained with Oil Red O in culture medium (A), DM with 0 μM (B), 1 μM (C), 2 μM (D), 4 μM (E), and 8 μM (F).

FIG. 4 illustrates berberine decreasing the amount of fat droplets in differentiated 3T3-L1 cells. CM: culture medium, DM: differentiation medium (includes differentiation medium 1 and 2), 1, 2, 4, 8 μM: DM with 1, 2, 4, 8 μM Berberine.

FIG. 5 (A) illustrates the effects of berberine on mRNA expression of PPARγ in 3T3-L1 cells. Lane 1: cells in culture medium (CM); Lane 2: Cells in differentiation medium (DM, includes differentiation medium 1 and 2); Lane 3-6: DM added with different concentrations of Berberine. (B), Relative mRNA expression of PPARγ compared to β-actin, values were expressed as means±S.D. (n=3). * denotes significant difference compared with DM group (p<0.05). (C), Effects of Berberine on mRNA expression of C/EBPα in 3T3-L1 cells. Lane 1: cells in culture medium (CM); Lane 2: cells in differentiation medium (DM); Lanes 3-6: Cells in DM added with different concentrations of Berberine. (D), Relative mRNA expression of C/EBPα compared to β-Actin, values were expressed as means±S.D. (n=3). * denotes significant difference compared with DM group (p<0.05).

FIG. 6 (A) illustrates the effects of berberine on relative mRNA expression of GATA-2 in 3T3-L1 cells. Lane 1: cells in culture medium (CM); Lane 2: Cells in differentiation medium (DM, includes differentiation medium 1 and 2); Lanes 3-6: DM with different concentrations of Berberine.

FIG. 6 (B) illustrates the relative mRNA expression of GATA-2 compared to β-Actin, values were expressed as means±S.D. (n=3). * denotes significant difference compared with DM group (p<0.05).

FIG. 6 (C) illustrates the effects of berberine on relative mRNA expression of GATA-3 in 3T3-L1 cells. Lane 1: cells in culture medium (CM); Lane 2: Cells in differentiation medium (DM); Lane 3-6: Cells in DM with different concentrations of Berberine.

FIG. 6 (D) illustrates the relative mRNA expression of GATA-3 compared to β-Actin, values were expressed as means±S.D. (n=3). * denotes significant difference compared with DM group (p<0.05).

FIG. 7 illustrates the effects of berberine on protein expression of GATA-2 and 3 in 3T3-L1 cells. A) illustrates protein bands of GATA-2 and β-Actin according to an embodiment of the present invention. Lane 1: cells in culture medium (CM); Lane 2: Cells in differentiation medium (DM), includes differentiation medium 1 and 2); Lanes 3-6: Cells in DM added with different concentrations of berberine. Values were expressed as means±S.D. * denotes significant difference compared with DM group (p<0.05). B) illustrates the relative protein expression of GATA-2 compared to β-Actin, C) illustrates protein bands of GATA-3 and β-Actin. D) illustrates relative protein expression of ratio of GATA-3 compared to β-Actin. Lane 1: cells in culture medium (CM); Lane 2: differentiation medium (DM); Lanes 3-6: Cells in DM added with different concentrations of Berberine. Values were expressed as means±S.D. * denotes significant difference comparing with DM group (p<0.05).

FIG. 8 illustrates the chemical structure of berberine chloride (A).

FIG. 9 illustrates the effects of clozapine and its combination of berberine (A), risperidone and its combination of berberine (B) on cell viabilities of 3T3-L1 during differentiation. Values given are the means±S.D. (n=8).

FIG. 10 illustrates the Oil-Red-O staining of 3T3-L1 cells after differentiation induction for 8 days and treated with vehicle (A), 15 μM clozapine (B), 15 μM clozapine and 8 berberine (C); and vehicle (D), 25 μM risperidone (E), 50 μM risperidone and 8 μM berberine (F). Quantification of Oil-Red-O staining in clozapine and berberine treatments was shown in FIG. G, quantification of Oil Red stain in risperidone and berberine treatments was shown in FIG. H. Values given are the means±S.D. (n=3). * denotes significant difference comparing with control group without berberine treatment (p<0.05). ** denotes significant difference comparing with no berberine treated group (p<0.05).

FIG. 11 illustrates the effects of clozapine and its combination of berberine (A), risperidone and its combination of berberine (B) on mRNA expression of SREBP-1, PPARγ, C/EBPα, LDLR, Adiponectin, and FAS relative to 3-actin during 3T3-L1 differentiation. Values given are the means±S.D. (n=3). * denotes significant difference comparing with control group without berberine treatment (p<0.05). ** denotes significant difference comparing with no berberine treated group (p<0.05).

FIG. 12 illustrates the effects of clozapine and its combination of berberine (A), risperidone and its combination of berberine (C) on protein expression of SREBP-1, PPARγ, C/EBPα relative to β-actin during 3T3-L1 differentiation and their quantification respectively (B, D), values given are the means±S.D. (n=3). * denotes significant difference comparing with control group (p<0.05). ** denotes significant difference comparing with only clozapine or risperidone treated groups (p<0.05).

FIG. 13 illustrates the chemical structure of berberine chloride (A) and evodiamine (B).

FIG. 14 illustrates the effects of berberine (A), evodiamine (B) and their combination (C, D) on cell viabilities of HWP during differentiation. Values given are the means±S.D. (n=4). * denotes significant difference comparing with only evodiamine treated cultures (p<0.05).

FIG. 15 illustrates that berberine appears to inhibit HWP differentiation after differentiation induction for 15 days. Oil-Red-O stains were done with cells cultured in Growth medium (GM, A), Differentiation and Nutrition medium (DM and NM, B), DM and NM with 1 μM (C), 2 μM (D), 4 μM (E), 8 μM (F) Berberine. Quantification was shown in FIG. G, values given are the means±S.D. (n=3).

FIG. 16 illustrates that evodiamine appears to inhibit HWP differentiation after differentiation induction for 15 days. Oil-Red-O stains were done with cells cultured in Growth medium (GM, A), Differentiation and Nutrition medium (DM and NM, B), DM and NM with 0.5 μM (C), 1 μM (D), 2 μM (E), 4 μM (F) evodiamine. Quantification was shown in FIG. G, values given are the means±S.D. (n=3).

FIG. 17 illustrates the quantification of lipids amount in differentiated HWP cultured in GM, DM and NM without or with various concentration of combination (A: 4 μM berberine+0, 0.5, 1, 2, 4 μM evodiamine, B: 8 μM berberine+0, 0.5, 1, 2, 4 μM evodiamine) for 15 days. Values given are the means±S.D. (n=3). * denotes significant difference comparing with only evodiamine treated cultures (p<0.05).

FIG. 18 illustrates the effects of berberine (A) and evodiamine (B) on mRNA expression of GATA-2, GATA-3, PPARγ and C/EBPα during HWP differentiation. Values given are the means±S.D. (n=3). * denotes significant difference comparing with vehicle treated cultures (p<0.05).

FIG. 19 illustrates the effects of berberine on protein expression of GATA-2 (A), GATA-3 (B), PPARγ (C) and C/EBPα (D) during HWP differentiation and the quantification (E), values given are the means±S.D. (n=3). * denotes significant difference comparing with vehicle treated cultures (p<0.05).

FIG. 20 illustrates the effects of evodiamine on protein expression of GATA-2 (A), GATA-3 (B), PPARγ (C) and C/EBPα (D) during HWP differentiation and the quantification (E), values were expressed as means±S.D. (n=3). * denotes significant difference comparing with vehicle treated cultures (p<0.05).

FIG. 21 illustrates the effects of berberine on mouse body weight and food intake. A: Body weight of control or berberine treated high-fat diet-induced obesity (fat diet, FD) mice and normal diet mice (ND). B: Body weight gain of control or berberine treated FD and ND mice after 36 days feeding and treatment. C: Average food intake amount of FD and ND mice in 36 days feeding and treatment. Food intake was recorded at 12 time points (every 3 days). Berberine (BB) concentration is 0.75, or 1.5, or 3 mg/kg/day. Data are means±S.D. (n=6 in A and B, n=12 in C). * denotes significant difference compared with vehicle treated FD mice subgroup (p<0.05).

FIG. 22 illustrates the effects of berberine on epididymal fat (A), liver (B), kidney (C), and spleen (D) weight relative to body weight in control or berberine treated high-fat diet-induced obesity (fat diet, FD) mice and normal diet mice (ND). Berberine (BB) concentration is 0.75, or 1.5, or 3 mg/kg/day. Data are means±S.D. (n=6). * denotes significant difference compared with control treated FD mice subgroup (p<0.05).

FIG. 23 illustrates the effects of berberine on serum concentration of glucose (A), triglyceride (B), and total cholesterol (C) in control or berberine treated high-fat diet-induced obesity (fat diet, FD) mice and normal diet mice (ND). Berberine (BB) concentration is 0.75, or 1.5, or 3 mg/kg/day. Data are means±S.D. (n=6). * denotes significant difference compared with control treated FD mice subgroup (p<0.05).

FIG. 24 illustrates the effects of berberine on mRNA expression of genes PPARγ, C/EBPα, GATA-2, and GATA-3 in control or berberine treated high-fat diet-induced obesity (fat diet, FD) mice. Gene expression is relative to β-actin and normalized by control treated subgroups. Berberine (BB) concentration is 0.75, or 1.5, or 3 mg/kg/day. Data are means±S.D. (n=6). * denotes significant difference compared with control treated FD mice subgroup (p<0.05).

FIG. 25 illustrates the effects of berberine on protein expression of genes PPARγ (A), C/EBPα (B), GATA-2 (C), GATA-3 (D), and β-actin (E) in control or berberine treated high-fat diet-induced obesity (fat diet, FD) mice. Relative protein expression is normalized by control treated subgroups and quantified (F). Berberine (BB) concentration is 0.75, or 1.5, or 3 mg/kg/day. Data are means±S.D. (n=3). * denotes significant difference compared with control treated FD mice subgroup (p<0.05).

FIG. 26 illustrates the effects of berberine, evodiamine and their combination on cell viabilities of RN46A cells. Values given are the means±S.D. (n=8). * denotes significant difference comparing with only evodiamine treated cultures (p<0.05).

FIG. 27 illustrates the effects of berberine, evodiamine, and their combination on mRNA expression of serotonin transporter of RN46A cells. Values given are the means±S.D. (n=3). * denotes significant difference comparing with vehicle treated cultures (p<0.05).

FIG. 28 illustrates the effects of berberine, evodiamine, and their combination on protein expression of serotonin transporter of RN46A cells.

FIG. 29a illustrates the effects of 100 μM berberine on serotonin transporter promoter activities. Berberine appeared to increase the promoter activities differently depending on different alleles.

FIG. 29b illustrates the effects of 2 μM evodiamine on serotonin transporter promoter activities. Evodiamine appeared to increase the promoter activities differently depending on different alleles.

FIG. 30a illustrates the quantification of Oil-Red-O staining In this embodiment, 3T3-L1 cells were induced to differentiate with differentiation medium and 15 μM clozapine (control) for 8 days. Varying concentration of berberine (2, 4, 8 μM) or its combination with 25 μM amentoflavone were added to observe the inhibitory effects on adipogenesis reflected by OD490 nm value, all values were normalized with control.

FIG. 30b illustrates the quantification of Oil-Red-O staining 3T3-L1 cells were induced to differentiate with differentiation medium (control) for 8 days. Varying concentration of berberine (2, 4, 8, 16 μM) or its combination with 25 μM amentoflavone were added to observe the inhibitory effects on adipogenesis reflected by OD490 nm value, all values were normalized with control.

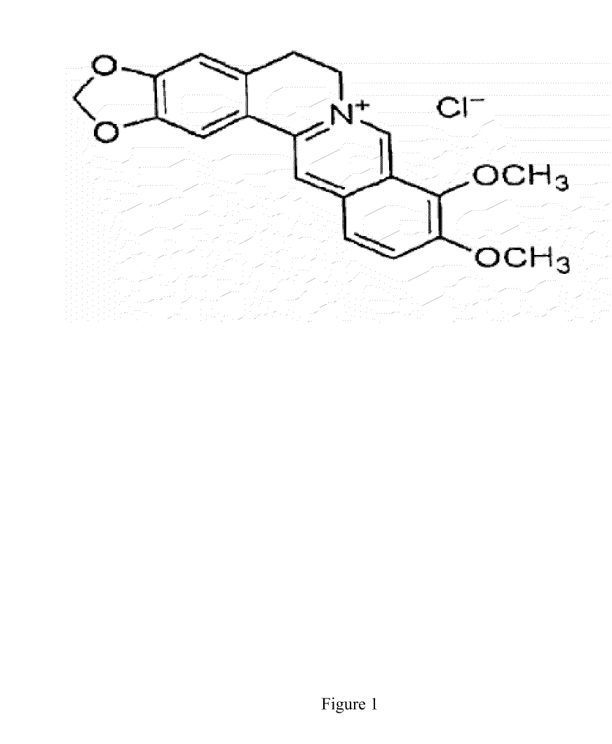

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

The term “analog” and its cognates refer to any molecule that demonstrates berberine activity.

Such molecule may be a salt of berberine, synthetic analog, fragment of berberine or endogenous biological molecule other than berberine capable of berberine-like activity. In sum, a berberine analog refers to any molecule that demonstrates bioactivity similar or greater than berberine itself.

The term “obesity” refers to an individual demonstrating any one or all of the symptoms and characteristics associated with obesity. Obesity is characterized by increased fat mass arising from the prolonged imbalance between energy intake and energy expenditure. Additionally, it may be caused by administration of a pharmacologic agent that causes this imbalance to occur and/or affects adipocyte differentiation in a manner that results in an increased fat mass.

Moreover, for the purposes of the present invention, the term “a” or “an” entity refers to one or more of that entity; for example, “a berberine analog” or “an analog” “refers to one or more of those compounds or at least one compound. As such, the terms “a” or “an”, “one or more” and “at least one” can be used interchangeably herein. It is also to be noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated or biologically pure berberine is a compound that has been removed from its natural milieu. As such, “isolated” and “biologically pure” do not necessarily reflect the extent to which the compound has been purified. An isolated compound of the present invention can be obtained from its natural source, can be produced using molecular biology techniques or can be produced by chemical synthesis.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

### Berberine Analogs

In certain embodiments, berberine analogs are utilized. Examples of berberine analogs that may be utilized in the present invention include salts of berberine such as berberine chloride, berberine phosphate, berberine sulphate, berberine bi-sulphate, berberine tannate, berberine hemisulphate, berberine citrate, or compounds with very similar chemical classification as berberine such as Sanguinarine, Coptisine, and Goldenseal.

In accordance with the disclosure, berberine analogs may be berberine fragments. Such fragments may be chemically synthesized or derived by any known means. Berberine fragments of the present invention retain bioactivity similar to or greater than berberine.

In another aspect, berberine analogs are synthetic berberine molecules that retain berberine bioactivity. Such analog molecule is capable of acting in a manner similar to endogenous berberine. Analogs of this type may be derivatives of berberine or have completely new molecular structures.

The skilled artisan will realize that the compounds listed above are exemplary only and that many variations may be used, depending on the particular berberine analog utilized, and the desired physiological effect. Such variations are known in the art.

In one embodiment, a composition is disclosed including, but not limited to, berberine or a berberine analog and a pharmacologic agent selected from the group consisting of an antipsychotic, a tranquilizer, an antidepressant, and an anticonvulsant. In one aspect, the pharmacologic agent is an antipsychotic. In a related aspect, the pharmacologic agent is clozapine or risperidone. In another related aspect, the pharmacologic agent does not induce fatty acid synthase expression.

In another aspect, the composition as disclosed includes a natural product. In a related aspect, the natural product is amentoflavone.

### Treatment or Prevention of Obesity

In treating or preventing obesity, according to the present disclosure, a therapeutically effective amount of berberine, berberine analogs or combinations thereof is administered to an individual susceptible to gaining weight, or one predisposed to developing obesity or one currently obese. Alternatively, any of the berberine treatments may precede or follow one or more of the other agent's treatment by intervals ranging from minutes to weeks. In some embodiments where the other agent and any of the berberine or berberine analogs are administered separately, one would generally ensure that a significant period of time did not expire between the time of each delivery, such that the agent and berberine or berberine analog would still be able to exert an advantageously combined (e.g., synergistic) effect on the patient. In such instances, it is contemplated that one would administer the various therapeutics simultaneously, within about 12-24 hours of each other, and, more particularly, within about 6-12 hours of each other. In some situations, it may be desirable to extend the duration of treatment with just the therapeutic agent, for example, where several days (2, 3, 4, 5, 6, or 7) to several weeks (1, 2, 3, 4, 5, 6, 7 or 8) lapse between the respective administrations.

There are also a wide variety of pharmacologic agents, including sedatives, tranquilizers, antidepressants, antipsychotics and anticonvulsants that, when prescribed, cause weight gain and obesity in the patients that take them. In addition, many interact with other medications, making administration of therapeutics a balancing act in order to prevent a toxic reaction or other serious medical conditions.

In light of the side-effect of weight gain and obesity related to a variety of pharmacologic agents, they may be administered in combination with berberine or berberine agonists to prevent or treat obesity. Agents suitable for use in combination therapy are any chemical compound or treatment method useful to patients gaining weight or those at risk of gaining weight or those with obesity or at risk of developing it. Such agents and factors include, but are not limited to, sedatives, tranquilizers, antidepressants, antipsychotics or anticonvulsants.

The berberine or berberine agonists may also be administered in combination with one or more natural products to prevent or treat weight gain and obesity. Natural products suitable for use in combination therapy are any chemical compound or treatment method useful to patients gaining weight or those at risk of gaining weight or those with obesity or at risk of developing it.

In one embodiment of the present disclosure, a method for treating or preventing obesity in a subject in need thereof is disclosed, including, but not limited to, administering to the subject a therapeutically effective amount of berberine, berberine salt, berberine analog or combination thereof, where the subject consumes a high fat diet or where said subject has be exposed to a pharmacologic agent which induces adipogenesis as a side effect. In one aspect, the administering suppresses the appetite of said subject.

In some aspects, the method as disclosed includes the use of a berberine salt selected from berberine chloride, berberine phosphate, berberine sulphate, berberine bi-sulphate, berberine tannate, berberine hemisulphate, and berberine citrate. In other aspects, the method as disclosed uses a berberine analog selected from an alkaloid extracted from Sanguinarine, Coptisine, or Goldenseal.

In one aspect, the pharmacologic agent includes, but is not limited to, a sedative, an antipsychotic, a tranquilizer, an antidepressant, and an anticonvulsant. In a related aspect, the pharmacologic agent is an antipsychotic. In another aspect, the antipsychotic does not induce expression of fatty acid synthase (FAS). In a related aspect, the antipsychotic is clozapine or risperidone.

In some aspects, a natural product is also administered. In a related aspect, the natural product is amentoflavone.

In another embodiment, a method for treating or preventing obesity in a subject is disclosed including administering a therapeutically effective amount of a combination of berberine, berberine salt, or berberine analog and a natural product to a subject in need thereof, where the subject has been exposed to a pharmacologic agent which induces adipogenesis as a side effect. In one aspect, the combination enhances the inhibition of adipogenesis in the subject such that the amount of berberine, berberine salt, or berberine analog administered which is necessary to achieve said inhibition is reduced relative to administration of berberine, berberine salt, or berberine analog alone.

In some aspects, the berberine salt includes, but is not limited to, berberine chloride, berberine phosphate, berberine sulphate, berberine bi-sulphate, berberine tannate, berberine hemisulphate and berberine citrate.

In other aspects, the berberine analog is an alkaloid extracted from Sanguinarine, Coptisine, or Goldenseal.

In one aspect, the pharmacologic agent includes, but is not limited to, a sedative, an antipsychotic, a tranquilizer, an antidepressant, and an anticonvulsant. In a related aspect, the pharmacologic agent is an antipsychotic. In a further related aspect, the antipsychotic does not induce expression of fatty acid synthase (FAS). In another related aspect, the antipsychotic is clozapine or risperidone.

In one aspect, the natural product is amentoflavone.

### Pharmaceutical Compositions and Routes of Administration

An aqueous solution of a therapeutically effective amount of berberine or berberine analogs alone or in combination with other compounds, dissolved or dispersed in a pharmaceutically acceptable carrier or aqueous medium, can be administered to an individual in order to treat behavioral characteristics associated with autism. The phrases “pharmaceutically or pharmacologically acceptable” refer to molecular entities and compositions that do not produce an unacceptably frequent adverse, allergic or other untoward reactions adverse, allergic or other untoward reaction when administered to an animal, or a human, as appropriate.

Aqueous solutions to be administered in accordance with the methods of the present invention comprise an effective amount of the compound, dissolved or dispersed in a pharmaceutically acceptable carrier or aqueous medium. Such compositions can also be referred to as inocula. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA and other regulatory agency standards.

The active berberine or berberine analogs, alone or in combination with other compounds will generally be formulated for parenteral administration, e.g., formulated for injection via the intravenous, intramuscular, sub-cutaneous, or intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid. It must be stable under the conditions of manufacture and storage and must be preserved against the contaminating action of microorganisms, such as bacteria and fungi.

Solutions of the active berberine or berberine analog compounds alone or in combination with other compounds effective in accordance with the method of the instant disclosure can be prepared in water suitably mixed with a surfactant, such as hydroxypropylcellulose. Dispersions can also be prepared in glycerol, liquid polyethylene glycols, and mixtures thereof, and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. In the case of microparticles, an aqueous suspending medium may optionally contain a viscosity enhancer such as sodium carboxymethylcellulose and optionally a surfactant such as Tween-20. The prevention of the action of microorganisms can be brought about by various antibacterial and antifungal agents, for example, parabens, chlorobutanol, phenol, sorbic acid, thimerosal, and the like. In many cases, it isotonic agents may be included such as, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent may result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like may also be employed.

For parenteral administration in an aqueous solution, for example, the solution should be suitably buffered if necessary and the liquid diluent first rendered isotonic with sufficient saline or glucose. These aqueous solutions are suitable for intravenous, intramuscular, subcutaneous and intraperitoneal administration. In this connection, sterile aqueous media that may be employed will be known to those of skill in the art in light of the present disclosure. For example, a unit dose could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580, herein incorporated by reference in its entirety).

The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. The person responsible for administration will, in any event, determine the appropriate dose for the individual subject. Activity of berberine is expressed in terms of USP units, as defined in a bioassay of uterine-stimulating potency of posterior pituitary extracts. One USP unit is the equivalent of approximately 2 μg of pure peptide.

The active therapeutic agents may be formulated within a mixture to comprise about 0.0001 to 1.0 milligrams, or about 0.001 to 0.1 milligrams, or about 1.0 to 100 milligrams or even about 0.01 to 1.0 grams per dose or so. Multiple doses can also be administered.

In addition to the berberine or berberine analog compounds formulated for parenteral administration, such as intravenous, subcutaneous or intramuscular injection, other alternative methods of administration of the present invention may also be used, including but not limited to intradermal administration (See U.S. Pat. Nos. 5,997,501; 5,848,991; and 5,527,288, herein incorporated in their entireties), pulmonary administration (See U.S. Pat. Nos. 6,361,760; 6,060,069; and 6,041,775, herein incorporated in their entireties), buccal administration (See U.S. Pat. Nos. 6,375,975; and 6,284,262, herein incorporated in their entireties), transdermal administration (See U.S. Pat. Nos. 6,348,210; and 6,322,808, herein incorporated in their entireties) and transmucosal administration (See U.S. Pat. No. 5,656,284, herein incorporated in its entirety). All such methods of administration are well known in the art.

One may also use intranasal administration in accordance with the present disclosure, such as with nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations, which are suitable for other modes of administration, include suppositories and pessaries. A rectal pessary or suppository may also be used. Suppositories are solid dosage forms of various weights and shapes, usually medicated, for insertion into the rectum or the urethra. After insertion, suppositories soften, melt or dissolve in the cavity fluids.

For suppositories, traditional binders and carriers generally include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain embodiments, oral pharmaceutical compositions will comprise an inert diluent or assimilable edible carrier, or they may be enclosed in a hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent, methyl and propylparabens as preservatives, a dye and flavoring, such as cherry or orange flavor.

In addition, alternative suitable compositions of the present invention may be used, including but not limited to hydrogels (See U.S. Pat. Nos. 6,372,813; 6,372,248; and 6,367,929, herein incorporated by reference in their entireties), vaginal rings (See U.S. Pat. Nos. 6,103,256; and 5,788,980, herein incorporated by reference in their entireties), patches (See U.S. Pat. Nos. 6,238,284; and 5,736,154, herein incorporated in their entireties), crystals (See U.S. Pat. No. 5,827,531, herein incorporated in its entirety), gels (See U.S. Pat. No. 5,830,506, herein incorporated in its entirety), liposomes (See U.S. Pat. Nos. 6,339,069; and 6,143,716, herein incorporated in their entireties) and implants (See U.S. Pat. Nos. 6,251,418; and 5,874,098, herein incorporated in its entirety). All such compositions are well known in the art.

In one embodiment, the use of a composition including berberine or a berberine analog and a pharmacologic agent including, but not limited to, an antipsychotic, a tranquilizer, an antidepressant, and an anticonvulsant in the manufacture of a medicament for the treatment of obesity in a subject in need thereof is disclosed.

In one aspect, the composition further includes amentoflavone. In another aspect, the pharmacologic agent is an antipsychotic. In a related aspect, the antipsychotic does not induce expression of fatty acid synthase (FAS). In a further related aspect, the antipsychotic is clozapine or risperidone.

The following examples are included to demonstrate particular embodiments. However, those of skill in the art should, in light of the present disclosure, appreciate that many changes can be made in the specific embodiments which are described and still obtain a like or similar result without departing from the spirit and scope of the of the disclosure.

### EXAMPLES

### Information Pertaining to Berberine Compound Used in Examples

1. Formulation and Structure: Berberine (Chloride Form)

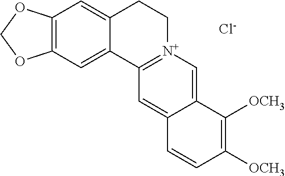

### Linear Formula: C20H18ClNO4 Molecular Weight: 371.81

2. Purity: Determined to be >98% by thin layer chromatography 3. Extraction: Berberine is a plant alkaloid presented in the roots, rhizomes, and stem bark of seven plant families including Coptis chinensis (Huanglian), Hydrastis anadensis and Coptis trifolia. Hydrastis anadensis has the most berberine among above plants. Significant differences in alkaloid content of Coptis chinensis (Huanglian), from its related American species. (Chinese Medicine 2009; 4: 1-4).
4. Administration: Oral and intraperitoneal (IP) administration were both utilized in the following Examples. In the high-fat diet-induced obesity mice experiment, the compound administration was 3 mg/kg/day berberine diluted in PBS.

### Example 1

### Berberine Appears to Increase Expression of GATA-2 and GATA-3 During Inhibition of Adipocyte Differentiation (Correlates to Paper 3-GATA-2 and GATA-3)

### Materials and Methods:

3T3-L1 cells, Dulbecco's Modified Eagle's Medium (DMEM) were purchased from ATCC Global Bioresource Center, Fetal Bovine Serum (FBS) was purchased from Atlanta Biologicals Corp., Berberine Chloride, Penicillin/streptomycin, Oil Red O, MTT, Isopropanol, Acrylamide/Bis-Acrylamide, DMSO, DNA size markers were purchased from Sigma Co., Trizol and Superscript One-Step RT-PCR kit were purchased from Invitrogen Life Technologies, Oligonucleotide primers were synthesized by Integrated DNA Technologies Inc., Antibodies were purchased from Santa Cruz Biotechnology Inc., Whatman nitrocellulose membrane was purchased from Thermo Fisher Scientific, ECL detection kit was purchased from GE Healthcare. Nonidet P40 was bought from Fisher Scientific Co.

Cell culture: 3T3-L1 cells were cultured at 37° C. in a humidified 5% CO₂ atmosphere and grown in a culture medium (CM) (DMEM supplemented with 10% FBS, 100 units/ml penicillin and 100 m/ml streptomycin). Differentiation conditions for 3T3-L1 were as described (Dowell et al, 2000). Briefly, confluent 3T3-L1 cells were maintained in culture medium for 48 hrs (day 0). Cells were placed in Differentiation Medium 1 (DM1) (CM with 167 nM insulin, 520 μM isobutylmethylxanthine (IBMX), and 1 μM dexamethasone) for a further 48 hrs (Day 2). The medium was then changed to differentiation medium 2 (DM2) (CM with 167 nM insulin) for a further 48 hrs (Day 4). The DM2 was then changed every 48 hrs for an additional 4 days (Day 8). Varying concentrations of Berberine were added to DM1 or DM2 in order to observe effects on cell viabilities during differentiation.

MTT assay: To detect the effect of Berberine on the viability of 3T3-L1 cells during proliferation, 3T3-L1 cells were plated in 96-well culture plates at a density of 6×10³ cells/well and cultured in CM or CM supplemented with varying concentrations of Berberine. Culture medium was removed at 24, 48 and 72 hrs and MTT (0.5 mg/ml in pure DMEM, 50 μl/well) was added. The plates were incubated at 37° C. for 4 hours, followed by addition of DMSO (150 μl/well), and incubated at 37° C. for 1 hour. Optical density was measured at 570 nm with 650 nm as background. The effect of Berberine on the viability of 3T3-L1 cells during differentiation was carried out using the methods described above and MTT assays were conducted on days 2, 4 6 and 8.

Oil-Red-O Staining: Oil-Red-O Staining was performed as previously described (Liu et al. 2004). Images were obtained using an Olympus IX70 inverted microscope (Tokyo, Japan). Quantification Oil-Red-O Staining of cells was carried out as described (Furuyashiki et al., Biosci Biotechnol Biochem (2004) 68:2253-2359).

RT-PCR Assay: Semi-quantitative reverse transcriptase polymerase chain reaction (RT-PCR) analysis was used to measure mRNA expression of PPARγ, C/EBPα, GATA-2 and GATA-3 in the control of f3-Actin. Briefly, 3T3-L1 cells were cultured in culture medium (CM), differentiation medium (DM, includes differentiation medium 1 and 2), or DM with varying concentrations of Berberine for 8 days. Total RNA was isolated with Trizol reagent according to the manufacturer's instruction. RNA was quantified at a ratio of 260 to 280 nm, and sample integrity was checked by 1.5% agarose gel electrophoresis. 1 μg of RNA from each sample was used for RT-PCR reaction using the Superscript One-Step RT-PCR kit following the manufacturers' instructions. The primer sequences were: PPARγ (Forward: 5′-TGA CCA TGG TTG ACA CCG-3′ (SEQ ID NO:1); Reverse: 5′-AAG CAT GAA CTC CAT AGT GG-3′ (SEQ ID NO:2)). C/EBPα (Forward: 5′-AAG GCC AAG AAG TCG GTG GA-3′ (SEQ ID NO:3); Reverse: 5′-CAG TTC ACG GCT CAG CTG TT-3′ (SEQ ID NO:4)). GATA-2 (Forward: 5′-TGC AAC ACA CCA CCC GAT ACC-3′ (SEQ ID NO:5); Reverse: 5′-CAA TTT GCA CAA CAG GTG CCC-3′ (SEQ ID NO:6)), GATA-3 (Forward: 5-TCT CAC TCT CGA GGC AGC ATG A-3′ (SEQ ID NO:7); Reverse: 5-GGT ACC ATC TCG CCG CCA CAG-3′ (SEQ ID NO:8)). β-Actin (Forward: 5′-CGC TCG TTG CCA ATA GTG-3′ (SEQ ID NO:9)); Reverse: 5′-GCT GTG CTA TGT TGC TCT AG-3′ (SEQ ID NO:10)).

The temperature cycling conditions of amplification were as follows: PPARγ, an initial step of 45° C. for 20 min, denaturation at 94° C. for 3 min, 30 cycles of denaturation at 94° C. for 1 min, annealing at 55° C. for 1 min, and elongation at 72° C. for 1 min, and a final extension at 72° C. for 8 min. CEBP/α, an initial step of 50° C. for 20 min, then denaturation at 94° C. for 2 min, 30 cycles of denaturation at 94° C. for 2 min, annealing at 58° C. for 2 min, and elongation at 72° C. for 2 min, and a final extension at 72° C. for 5 min. GATA-2, an initial step of 50° C. for 20 min, then denaturation at 94° C. for 2 min, 45 cycles of denaturation at 94° C. for 20 s, annealing at 58° C. for 20 s, and elongation at 72° C. for 1 min, and a final extension at 72° C. for 8 min. GATA-3, an initial step of 50° C. for 20 min, then denaturation at 94° C. for 3 min, 30 cycles of denaturation at 94° C. for 30 s, annealing at 51° C. for 30 s, and elongation at 70° C. for 30 s, and a final extension at 72° C. for 8 min. β-Actin, an initial step of 45° C. for 20 min, then denaturation at 94° C. for 3 min, 30 cycles of denaturation at 94° C. for 20 s, annealing at 55° C. for 30 s, and elongation at 72° C. for 1 min, and a final extension at 72° C. for 8 min. RT-PCR was performed using the Gene Amplify PCR System, the amplification products electrophoresis was carried out on 1.5% agarose gels, visualized by ethidium bromide (0.5 g/ml) staining, and photographed. Gel images were scanned using UVP image analysis system. The intensities of specific PCR bands were quantified in relation to β-Actin bands from the same sample.

Immunoblot Analysis: GATA-2 and GATA-3 protein expression were assessed by Western blot. 3T3-L1 cells were cultured in culture medium (CM), differentiation medium (DM, includes differentiation medium 1 and 2), or DM with various concentrations of Berberine for 8 days. The cells were washed twice with PBS, lysed in a lysis buffer (HEPES-Tx-PI buffer: 10 mM HEPES, 5 mM EDTA, 350 mM Sucrose, 1 ug/ml leupeptin, 1 ug/ml pepstatin, 1% Triton-X, 1 mM PMSF, diluted in water), processed with a 25G needle and centrifuged at 10,000 rpm for 5 min. Protein concentration was determined using the BSA kit according to the Pierce Protein Assay Protocol. Total protein (50 μg) was used for GATA-2 and GATA-3 analysis and was separated by electrophoresis using BioRad Electrophoretic System (60 V for 1 hour followed by 90 V for 5 hours). The protein was then transferred to nitrocellulose membranes at 35 V overnight. The membrane was blocked for 1 hour at room temperature in 5% nonfat milk in Tris-buffered saline (TBS), then washed with Tween-20-TBS (TTBS, 0.1% Tween-20) three times (15 min, 5 min, 5 min), followed by incubation with primary antibodies of GATA-2 (dilution 1:500), GATA-3 (dilution 1:500), β-Actin (dilution 1:1000) at room temperature for 2 hours. The membrane was rinsed three times (15 min, 5 min, 5 min), then incubated at room temperature for 1 hour with Horseradish peroxidase-conjugated second antibody (1:10,000). The membrane was then incubated in ECL reagent for 1 minute. Protein was visualized using UVP image analysis system.

Statistical analysis: The data was analyzed by the ANOVA procedure of Statistical Analysis System (SAS Institute, 1999-2001). Significant differences between groups were determined using Duncan's multiple range tests at the p<0.05 (*).

### Results:

A. Berberine Appeared to have Minor Effect on Viability of 3T3-L1 Cells.

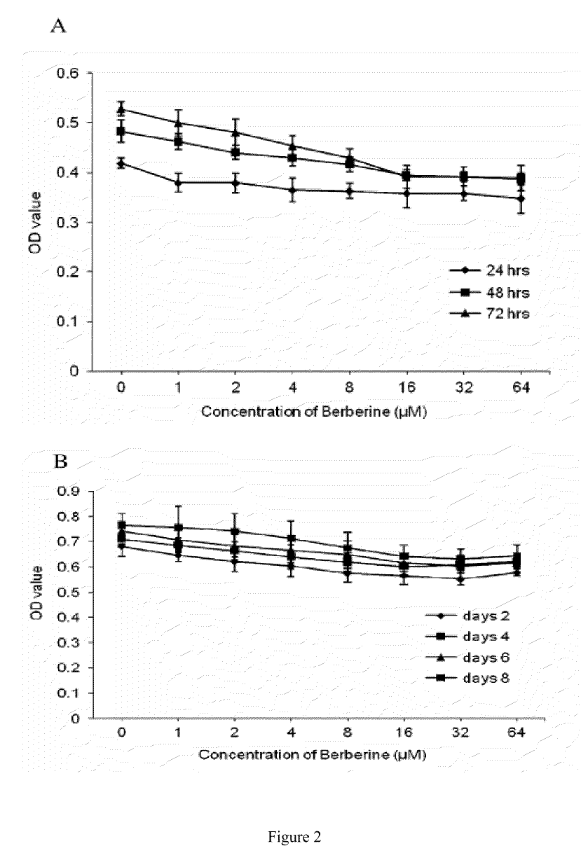

Berberine appeared to have a minor effect on the viability of 3T3-L1 cells during proliferation and differentiation. MTT analysis was carried out at day 1, 2 and 3 of proliferation and during days 2, 4, 6 and 8 of differentiation to detect the effect of Berberine on the viability of 3T3-L1 cells during proliferation (FIG. 2A) and differentiation (FIG. 2B). 3T3-L1 cells were treated with various concentrations of Berberine (0, 1, 2, 4, 8, 16, 32, 64 μM) during each stage. Berberine had a small effect on cell viability during proliferation with 8 μM berberine appearing to only decrease viability by 13.2%, 13.8% and 18.6% after 1, 2 and 3 days respectively of culture. During differentiation, 8 μM Berberine decreased cell viability by 15.6%, 12.8%, 12.6% and 11.4% following 2, 4, 6 and 8 days respectively of the induction of differentiation. These findings demonstrate that berberine appeared to only have a minor effect on cell viability.

### B. Berberine Appeared to Inhibit Adipogenesis of 3T3-L1 Cells as Measured by Quantitative Oil Red O Staining

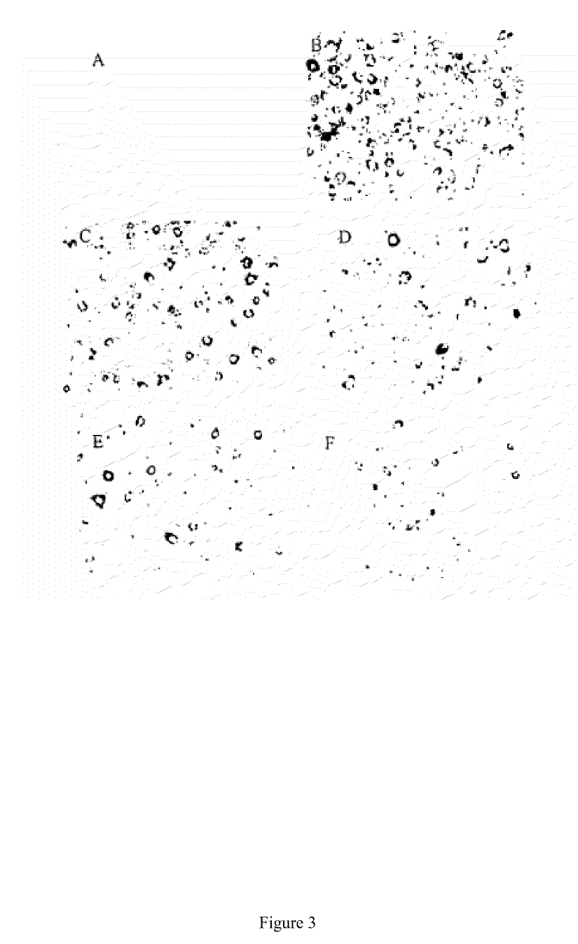

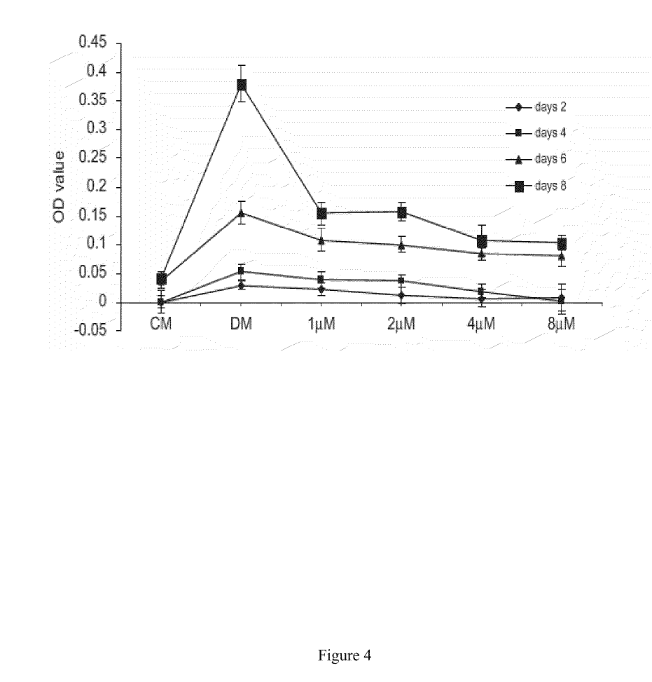

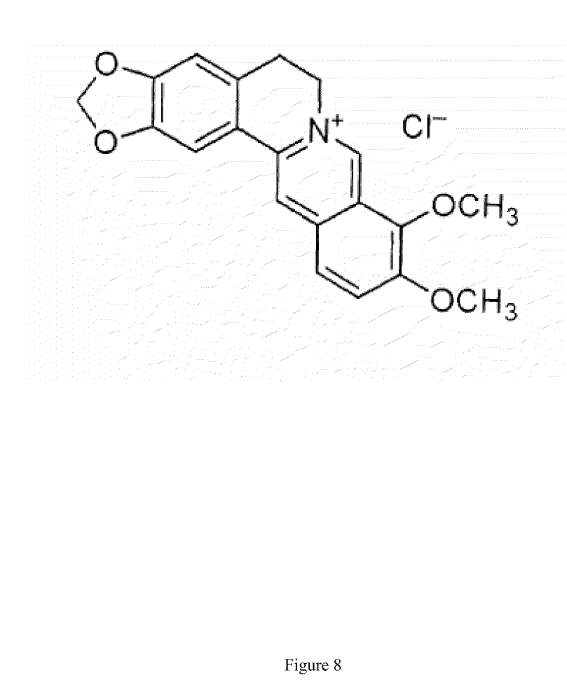

To investigate the effect of Berberine on the expression of GATA-2 and 3 during the inhibition of adipocyte differentiation, the Berberine inhibition of adipogenesis model established by Huang et al., 2006 was adapted for this experiment. Varying concentrations of Berberine (0, 1, 2, 4, 8 μM) were added to the culture medium during differentiation of 3T3-L1 cells. Standard Oil Red O staining was then used to examine the extent of differentiation process at days 2, 4, 6, and 8 using microscopy. FIG. 3 shows 3T3-L1 cells 8 days after differentiation. 3T3-L1 cells examined following 8 days, in normal cultured medium (FIG. 3A) did not stain by Oil Red O while almost all cells cultured in differentiated medium (DM) (FIG. 3B) stained red indicating almost total differentiation. Cells grown in DM with the addition of berberine in the concentration of 1 μM (FIG. 3C) and 2 μM (FIG. 3D), respectively, appeared to show inhibition of cellular differentiation. Moreover, cells grown in the presence of 4 μM (FIG. 3E) and 8 μM (FIG. 3F) berberine appeared to show the greatest suppression of differentiation. The inhibition of adipogenesis by Quantitative Oil Red O staining measurements and showed 3T3-L1 cells which were grown in 1, 2, 4, 8 μM Berberine and were 6 days post differentiation induction, appeared to have reduction in fat droplets of 30.6%, 35.8%, 40% and 48.6% respectively (FIG. 4). More strikingly in fully differentiated cells for 8 days, 1, 2, 4, and 8 Berberine appeared to inhibit the fat amount by 59.4%, 58.4%, 71.8% and 72.6% respectively. (FIG. 4).

### C. Berberine Appeared to Induce Down Regulation of Adipogenic Marker Genes.

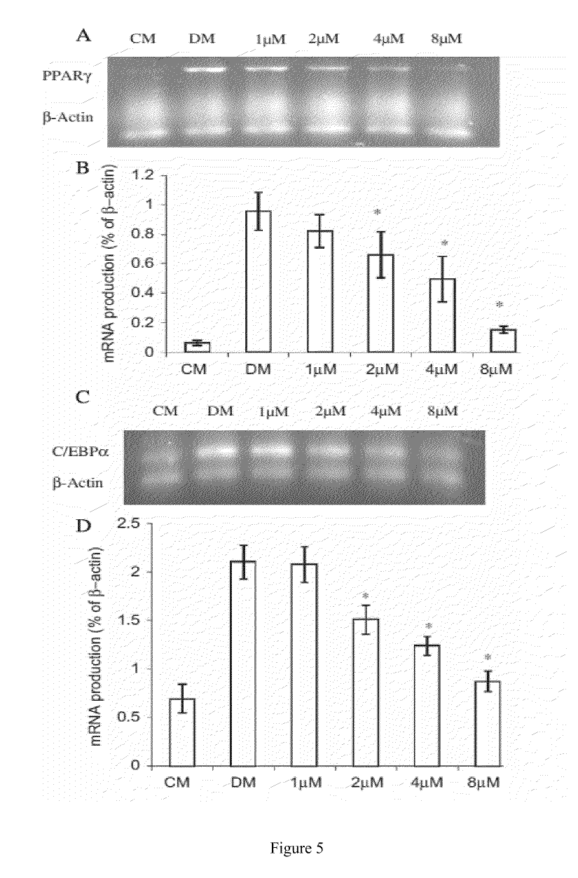

The adipogenic inhibition model was further validated by examining the mRNA expression of PPARγ and C/EBPα genes which are widely reported as being molecular markers in the adipocyte differentiation process (Huang et al., Biochem Biophys Res Comm (2006) 348:571-578; Tong et al., Science (2000) 5489:134-138; and Tong et al., Mol Cell Biol (2005) 25:706-715) and interact with both GATA-2 and 3 to control the pre-adipocyte to adipocyte transition. PPARγ mRNA expression was increased in differentiated cells but decreased following the addition of Berberine (FIG. 5A) relative to the expression of β-Actin (FIG. 5B). PPARγ mRNA expression was increased in cells cultured in differentiated medium compared to that of cells cultured in normal culture medium (0.957±0.128 vs. 0.061±0.016). When compared with the cells cultured in differentiated medium the addition of Berberine resulted in a decrease of relative PPARγ mRNA expression to 0.826±0.113 (1 μM), 0.662±0.155 (2 μM), 0.498±0.153 (4 μM), and 0.152±0.022 (8 μM). This shows that Berberine inhibited PPARγ expression in a dose dependent manner. Furthermore, the mRNA expression of C/EBPα was increased in differentiated cells and decreased after addition of Berberine (FIG. 5C) relative to the expression of 3-Actin (FIG. 5D). In addition C/EBPα mRNA expression was increased in cells cultured in differentiated medium compared to cells cultured in normal culture medium (2.099±0.175 vs. 0.693±0.149). However, and perhaps more importantly C/EBPα mRNA expression in cells cultured in differentiated medium and with the addition Berberine resulted in a dose dependent decrease in relative expression to 2.076±0.185 (1 μM), 1.509±0.149 (2 μM), 1.239±0.099 (4 μM), 0.874±0.106 (8 μM).

D. Berberine Appeared to Increase the mRNA Expression of GATA-2 and GATA-3 Genes During the Inhibition of Adipocyte Differentiation.

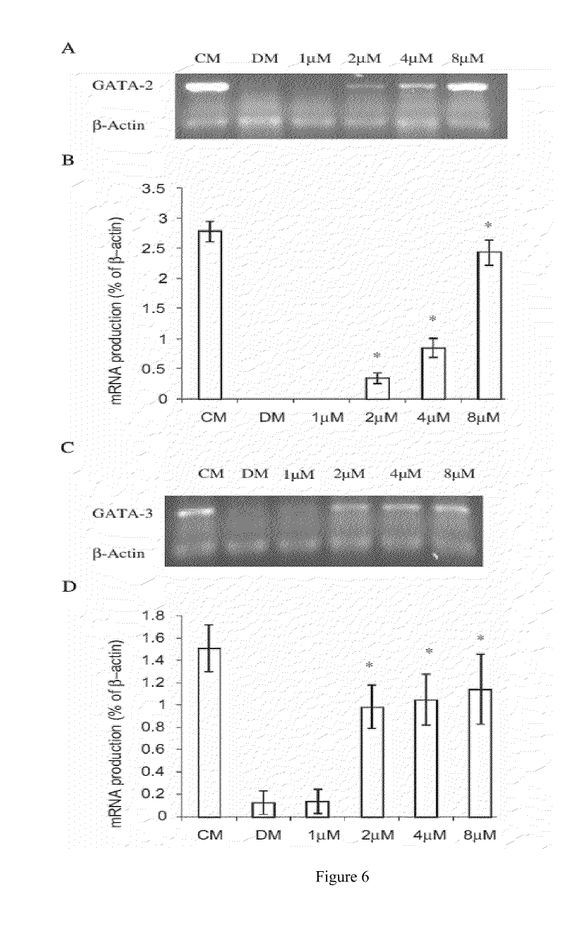

GATA-2 mRNA was only expressed in cells cultured in normal culture medium (FIG. 6A). No expression of GATA-2 mRNA in differentiated cells was observed. However, following the addition of increasing concentrations of Berberine, the GATA-2 mRNA expression levels appeared to increase. Furthermore, relative GATA-2 mRNA expression appeared to decrease in cells cultured in differentiated medium compared to that observed in cells cultured in normal culture medium (0 vs. 2.770±0.172) (FIG. 6B). More importantly, GATA-2 mRNA expression was increased in the cells cultured in differentiation medium with the addition of Berberine when compared to that of differentiation medium alone: 0.346±0.086 (2 μM), 0.844±0.159 (4 μM), 2.430±0.211 (8 μM), similarly to GATA-2, GATA-3.

mRNA was only expressed in cells cultured in normal culture medium and was not observed in differentiated cells (FIG. 6C). However, following the addition of increasing concentrations of Berberine to the media, GATA-3 mRNA expression also appeared to increase steadily. GATA-3 mRNA expression appeared to be decreased in cells cultured in differentiated medium compared to that in the cells cultured in normal culture medium (0.130±0.106 vs. 1.507±0.208) (FIG. 6D). Finally, when comparing differentiated cells grown with or without Berberine, it was observed that the addition of Berberine appeared to result in an increase in relative GATA-3 mRNA expression to 0.137±0.109 (1 μM), 0.985±0.192 (2 μM), 1.049±0.229 (4 μM), 1.143±0.312 (8 μM). These results show that Berberine appears to influence GATA-2 and GATA-3 expression during differentiation and induces increased gene expression of these genes in a dose dependent manner.

### E. GATA-2 and GATA-3 Protein Expression Appeared to be Increased in Cells Grown in the Presence of Berberine.

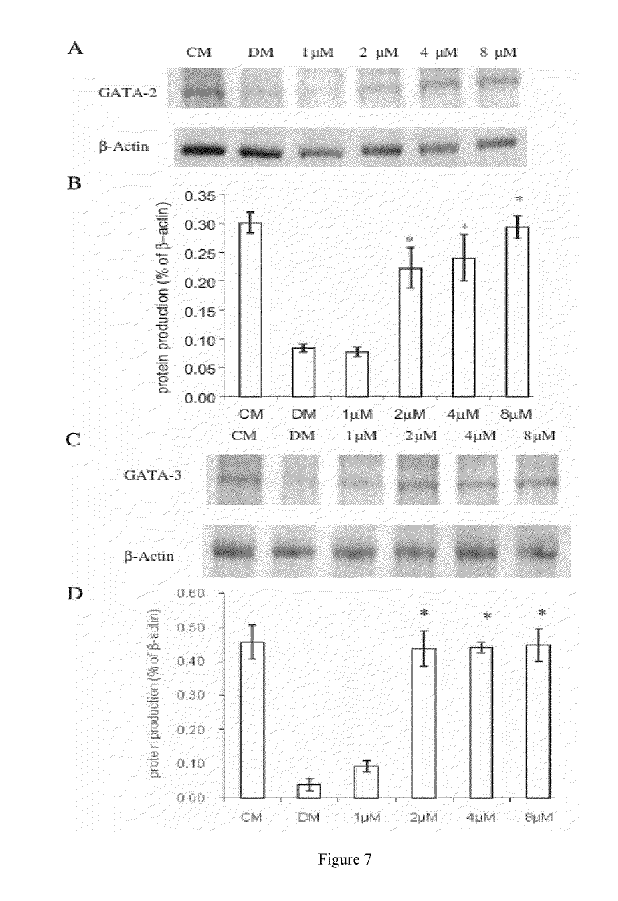

In order to investigate the correlation between GATA-2 and GATA-3 protein expression and the gene expression findings discussed above, the effect of Berberine treatment on total protein expression in 3T3-L1 cells was examined with Western blot analysis. The results from the protein studies appeared similar to GATA-2 mRNA expression, GATA-2 protein appeared to be expressed in cells cultured in normal culture medium while decreased in cells cultured in differentiated medium. Furthermore the addition of Berberine (1, 2, 4, 8 μM), appeared to increase GATA-2 protein expression (p<0.05)(FIGS. 7A and B).

There also appeared to be a similar expression pattern for the GATA-3 protein. GATA-3 protein appeared expressed in cells cultured in normal culture medium while decreased in cells cultured in differentiated medium. Furthermore, similar to GATA-2, the addition of Berberine (1, 2, 4, 8 μM) appeared to increase GATA-3 protein expression (p<0.05) (FIGS. 7C and D). Protein levels and concentration were confirmed by using β-Actin protein expression as a control in all experiments.

### Discussion:

Adipocyte differentiation appears to play a crucial role in obesity. The mechanisms of adipocyte differentiation have been extensively studied in pre-adipocyte culture systems with key transcription factors involved in the complex transcriptional cascade that occurs during adipocyte differentiation being identified. These include the adipogenic PPARγ and members of the C/EBP family which are major regulators of metabolic gene expression. It appears the compound Berberine has antidiabetic properties, and in vivo studies have indicated that the administration of Berberine demonstrates insulin sensitizing as well as weight- and lipid lowering effects in both db/db mice and in high fat-fed rats. Furthermore, an inhibitory effect on adipocyte differentiation has been observed. Therefore, the investigation of the molecular action of Berberine is important in considering its use as a possible anti-diabetic and anti-obesity agent. In this study, it appears that Berberine has a minor inhibitory effect on the viability of 3T3-L1 cells during proliferation and differentiation; 8 μM Berberine appears to decrease the cell viability by 18.6% when cultured in culture medium for 3 days, while, in contrast, 8 μM Berberine appeared to decrease the cell viability by 11.4% in cells cultured in differentiation medium for 8 days. Since 3T3-L1 cells proceed through two rounds of cell mitotic division at the beginning of differentiation induced by differentiation medium and suppression of this process appears to abort the differentiation process, the slight inhibition of cell viability indicates low toxicity of Berberine and aids in the inhibition of differentiation.

The Oil red 0 staining demonstrates vividly the generation of fat in 3T3-L1 cells, and quantitatively and comprehensively indicated that Berberine appeared to decrease the amount of fat present in these cells (8 μM Berberine decreased fat amount by 72.6% after 8 days). The GATA family of transcriptional regulators plays a key role in adipogenesis. Two isoforms, GATA-2 and GATA-3, are specifically expressed in murine pre-adipocytes but not in mature adipocytes. Moreover, continuous expression of GATA factors in pre-adipocyte cell lines appears to inhibit terminal differentiation into mature adipocytes. This experiment indicated that Berberine could increase the gene and protein expression of GATA-2 and 3 during pre-adipocyte 3T3-L1 cell differentiation thus appearing to influence the adipocyte differentiation process.

Additionally, it appears that Berberine decreases the expression of PPARγ and C/EBPα, but more importantly that it also appears to increase expression of GATA-2 and 3 both at the gene and protein level following Berberine treatment.

### Example 2

### Berberine Appears to Inhibit SREBP-1-Related Clozapine and Risperidone Induced Adipogenesis in 3T3-L1 Cells

### Materials and Methods

Cell culture and drug treatment: 3T3-L1 cells (American Type Culture Collection, Manassas, Va., USA) were cultured at 37° C. in a humidified 5% CO₂ atmosphere and grown in a DMEM (Sigma-Aldrich, St. Louis, Mo. USA) supplemented with 10% Fetal Bovine Serum (FBS, Atlanta Biologicals Corp., Lawrenceville, Ga., USA), 100 units/ml penicillin and 100 μg/ml streptomycin. Differentiation was induced by addition of 167 nM insulin, 520 μM isobutylmethylxanthine (IBMX), and 1 μM dexamethasone for 2 days and then only with 167 nM insulin for 6 days. Detailed protocol was described as our previous report (Hu et al., Phytomedicine (2009) 16(9):864-873). Varying concentrations of clozapine, risperidone, and berberine (All ordered from Sigma-Aldrich, USA) were added to differentiation medium at the beginning of differentiation induction for 8 days to observe their effects.

MTT assay: To detect the effect of clozapine, risperidone and their combination with berberine on the viabilities of 3T3-L1 during differentiation induction, 3T3-L1 cells were plated in 96-well culture plates and induced differentiation using methods mentioned above, varying concentrations of clozapine, risperidone, and 8 μM berberine were added to differentiation medium. Medium was removed after differentiation and treatment for 8 days, MTT (Methylthiazolyldiphenyl—tetrazolium bromide 0.5 mg/ml, 50 μl/well) was added. The plates were incubated at 37° C. for 4 hours, followed by the addition of DMSO (150 μl/well), and incubated at 37° C. for 1 hour. Optical density (OD) was measured at 570 nm with 650 nm as background.

Oil-Red-O staining and quantification: Oil-Red-O Staining and its quantification were performed as previously reported (Liu et al., Sinica (2004) 25:1052-1057). Briefly, medium was removed and cells were washed with PBS twice, fixed with 3.7% formalin at room temperature for 30 min, then rinsed with pure water, added 60% isopropanol and incubated for 5 minutes, then moved out isopropanol and stained cells with diluted Oil-Red-O solution (Oil-Red-O store solution (3 mg/ml in pure isopropanol): water=3:2) at room temperature for 10 min. Then the diluted Oil-Red-O solution was removed and cells were washed 3 times with pure water. Images were obtained using an Olympus IX70 inverted microscope equipped for phase-contrast microscopy (Olympus, Tokyo, Japan). After staining, the cells were washed twice with 70% ethanol to remove excess stain. Stained oil droplets in the cells were dissolved in isopropanol containing 4% Nonidet-P40 (Fisher Scientific, Pittsburgh, Pa., USA) shaking in 37° C. at speed 100 rpm for 1 hour, and OD values were measured at an absorbance of 490 nm and then normalized with control group.

Realtime RT-PCR: Real-time reverse transcriptase polymerase chain reaction (RT-PCR) analysis was used to measure mRNA expression of genes SREBP-1, PPARγ, C/EBPα, low density lipoprotein receptor (LDLR), Adiponectin, and fatty acid synthase (FAS) under the control of β-actin. Briefly, total RNA was isolated from 3T3-L1 cells following treatment for 8 days with Trizol reagent (Invitrogen, Carlsbad, Calif., USA) according to the manufacturer's instruction. RNA was quantified using absorption of light at 260 nm, and sample purity was checked by OD 260 nm/280 nm. 0.8 μg of total RNA from each sample was used for reverse transcription reaction using the TaqMan Reverse Transcription Reagents (Applied Biosystems, Foster City, Calif., USA) according to the manufacturers' instructions. PCR was done using the SYBR green Master Mix (Applied Biosystems, Foster City, Calif., USA) according to the manufacturers' instructions. The primers (Integrated DNA Technologies, Coralville, Iowa, USA) sequences are shown in Table 1. The temperature cycling conditions of amplification were as follows: an initial step of denaturation at 95° C. for 10 min, 40 cycles of denaturation at 95° C. for 30 s, annealing at 55° C. for 30 s, and elongation at 72° C. for 30 s. Realtime RT-PCR was performed using Mx3000P Realtime Thermocyclers (Statagene, La Jolla, Calif., USA). The relative mRNA levels of these genes were calculated by 2-delta delta CT method and normalized with control groups.

Immunoblotting: Protein expression of SREBP-1, PPARγ, and C/EBPα relative to 3-actin was assessed by Western Blot. Total proteins were isolated from 3T3-L1 cells treated with compounds or vehicles for 8 days. The cells were washed twice with PBS, lysed in a HEPES-Tx-PI buffer (910 mM HEPES, 5 mM EDTA, 350 mM Sucrose, 1 μg/ml leupeptin, 1 μg/ml pepstatin, 1% Triton-X, 1 mM PMSF), processed with 25G needles and centrifuged at 12,000 rpm for 5 min. Protein concentration was determined using the Micro BCA Protein Assay Kit (Fisher Scientific, Pittsburgh, Pa., USA) following the manufacturers instructions. 25 μg proteins were loaded and separated by electrophoresis using the BioRad Electrophoretic System (65 V for 7 hour). The proteins were then transferred to nitrocellulose membranes at 40 V overnight. The membrane was blocked for 1 hour at room temperature in 5% nonfat milk in Tris-buffered saline (TBS), then washed with Tween-20-TBS (TTBS, 0.1% Tween-20) three times (15 min, 5 min, 5 min), followed by incubation with primary antibodies SREBP-1 (dilution 1:200), PPARγ (dilution 1:200), C/EBPα (dilution 1:200) (all purchased from Santa Cruz Biotechnology, Santa Cruz, Calif., USA) and 3-actin (dilution 1:1000, Sigma-Aldrich, St. Louis, Mo., USA) at room temperature for 2 hours. The membrane was rinsed three times (15 min, 5 min, 5 min), then incubated at room temperature for 1 hour with Horseradish peroxidase-conjugated second antibody (1:10,000). The membrane was then incubated in ECL reagent (GE Healthcare, Piscataway, N.J., USA) for 45 seconds. Protein was visualized using the UVP image analysis system (UVP, Upland, Calif., USA).

Statistical analysis: The data was analyzed by the ANOVA procedure of Statistical Analysis System (SAS Institute, 1999-2001). Significant differences between groups were determined using Student's t-test or Duncan's multiple range tests at the p<0.05 (*).

### Results:

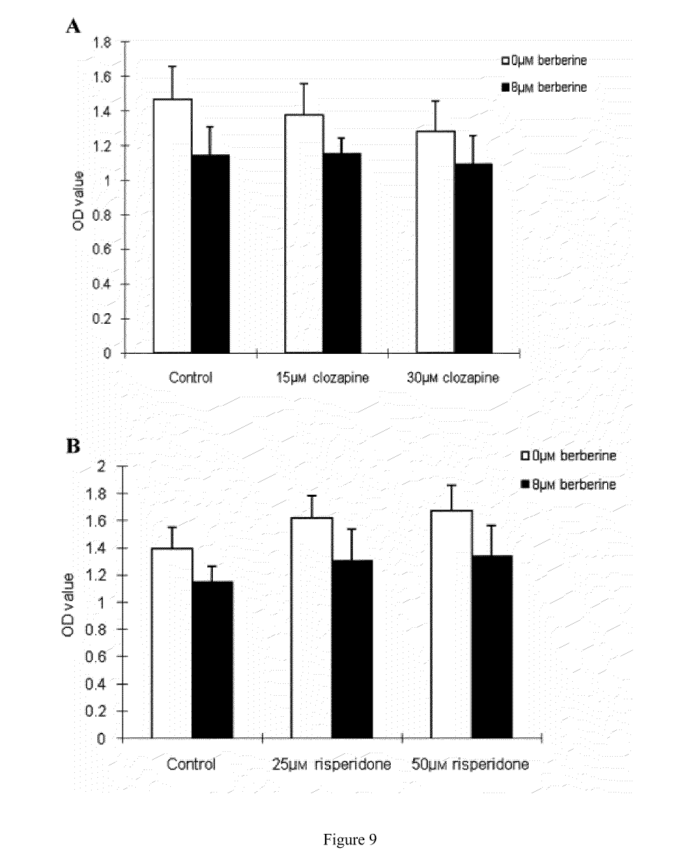

MTT assay: MTT analysis was carried out at day 8 of differentiation to detect the effect of clozapine, risperidone, and their combination with berberine on the viability of 3T3-L1 cells (FIGS. 9A and B). 3T3-L1 cells were treated with 15 μM or 30 μM clozapine, 25 μM or 50 μM risperidone, and their combination with 8 μM berberine. Clozapine appeared to reduce cell viability slightly (6.2% for 15 μM clozapine treatment and 12.5% for 30 μM clozapine treatment respectively), and risperidone appeared to enhance cell viability slightly (14% for 15 μM clozapine treatment and 16.8% for 30 μM clozapine treatment respectively). Comparing to no berberine treated groups, 8 μM berberine treatment appeared to decrease cell viability slightly by 22.3%, 16.5%, and 15% respectively when combined with 0 μM, 15 μM, 30 μM clozapine treatment. Comparing to no berberine treated groups, 8 μM berberine treatment appeared to decrease cell viability slightly by 17.8%, 19.5%, 20.2% respectively when combined with 0 μM, 25 μM, 50 μM risperidone treatment. However, all inhibition of cell viability was not statistically significant. This result indicated that clozapine, risperidone, and berberine appeared to have a minor effect on cell viability.

Oil-Red-O staining and quantification: In order to study the potential inhibitory effects of berberine on adipogenesis induced by clozapine and risperdone during differentiation, 8 μM berberine combined with various concentrations of clozapine (0, 15, 30 μM) and risperdone (0, 25, 50 μM) was added to the differentiation medium for 8 days during the culture of 3T3-L1. Differentiation was monitored by Oil-Red-O staining as previously described.

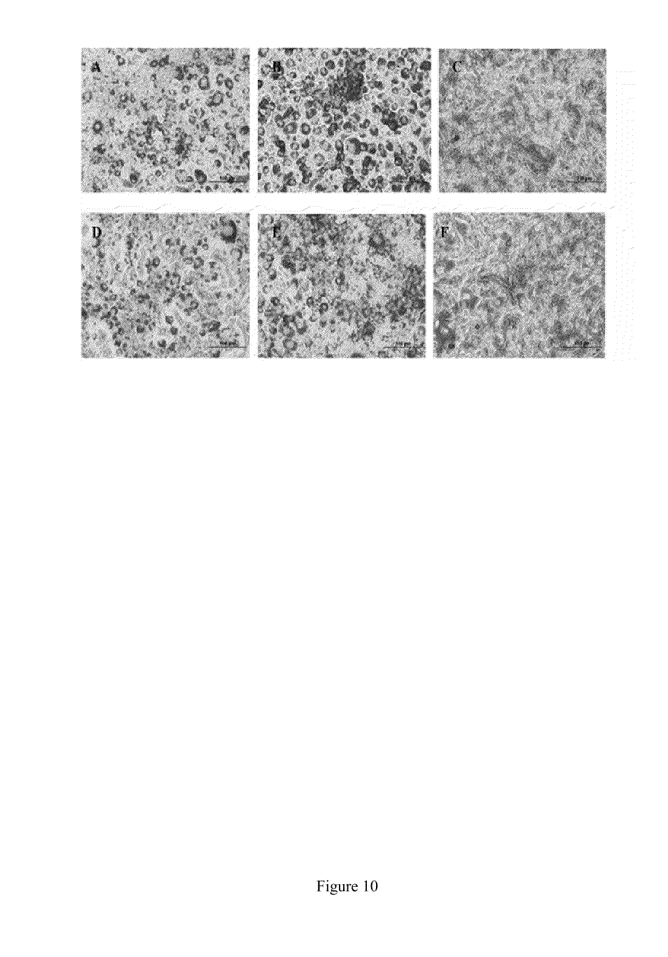

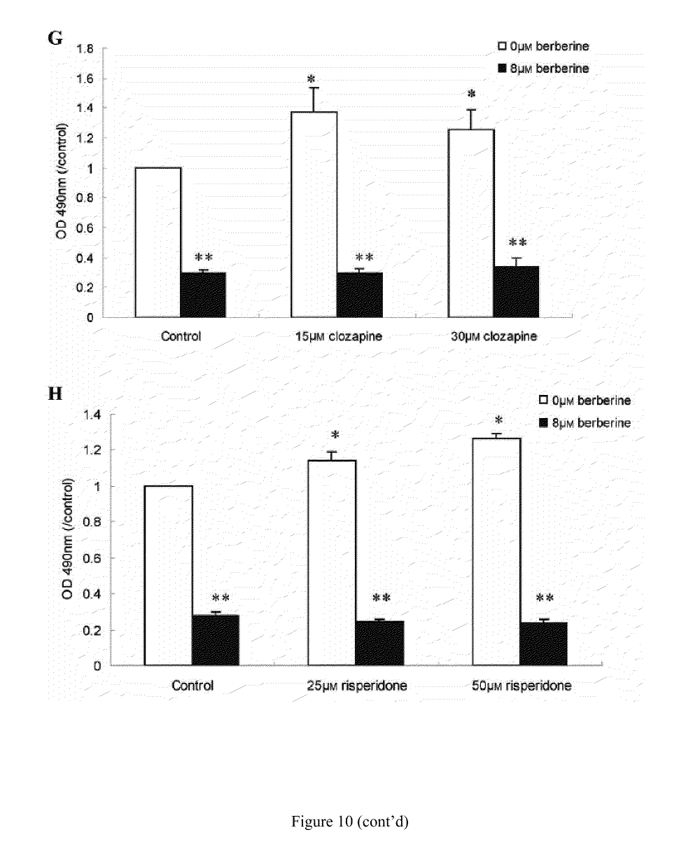

FIG. 3 indicates the Oil-Red-O staining of 3T3-L1 cells for 8 days treatment, most 3T3-L1 cells cultured in differentiation medium treated with vehicle (control group) became red-stained indicating good differentiation (FIG. 10A), and also as expected, the staining of 3T3-L1 cells treated with 15 μM clozapine appeared to reveal enhanced differentiation (FIG. 10B). However, cells grown in 15 μM clozapine and 8 μM berberine indicated deep suppression of differentiation (FIG. 10C). In addition, 30 μM clozapine appeared to reveal enhanced differentiation as well as the addition of 8 μM berberine indicated deep suppression either. Similar results were observed in risperidone and its combination with berberine treated groups and the staining of 3T3-L1 cells treated with 50 μM risperidone also appeared to reveal enhanced differentiation (FIG. 10F) comparing to the control group (FIG. 10E). However, cells grown in 50 μM risperidone and 8 μM berberine (FIG. 10G) indicated deep suppression of differentiation. In addition, 25 μM risperidone also appeared to reveal enhanced differentiation and the addition of 8 μM berberine also indicated deep suppression.

Quantitative Oil-Red-O staining measurements (FIG. 10H) indicated that 3T3-L1 cells grown in the presence of 15 μM clozapine and 30 μM clozapine for 8 days induced adipogenesis by 37.4% and 25.2% respectively comparing to vehicle treated group, 8 berberine treatment for 8 days indicated a reduction in lipid content by 70%, 78.5%, 73.2% in control, 15 μM clozapine and 30 μM clozapine groups respectively when compared with no berberine treated groups. Actually, the adipogenesis among control, 15 μM clozapine and 30 μM clozapine groups associated with 8 μM berberine treatment appeared to be the same (relative OD values are 0.3±0.02, 0.3±0.02, and 0.34±0.06 respectively).

Quantitative Oil-Red-O staining measurements (FIG. 10I) indicated that 3T3-L1 cells grown in the presence of 25 μM risperidone and 50 μM risperidone induced adipogenesis by 14.1% and 26.5% respectively comparing to vehicle treated group, 8 μM berberine treatment for 8 days had a reduction in lipid content by 72%, 78.7%, 81% in control, 25 μM risperidone and 50 risperidone groups respectively when compared with no berberine treated group. Actually, the adipogenesis among control, 25 μM risperidone and 50 μM risperidone groups associated with 8 μM berberine treatment appeared to be almost the same (relative OD values are 0.28±0.02, 0.24±0.01, and 0.24±0.02 respectively).

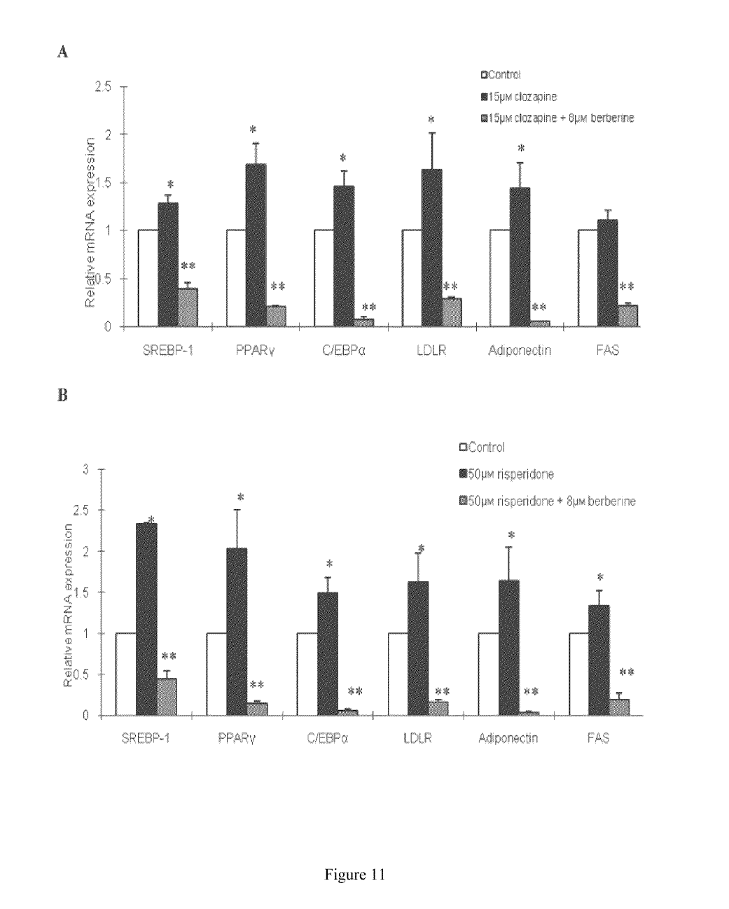

Real-time RT-PCR: In order to investigate the possible mechanisms for induced adipogenesis by clozapine or risperidone and the inhibitory effects from berberine, mRNA expression of related proteins from SREBP-1 pathway such as SREBP-1, PPARγ, C/EBPα, LDLR, Adiponectin, and FAS was tested by real-time RT-PCR (FIG. 11A, B).

When treated with 15 μM clozapine for 8 days during differentiation of 3T3-L1 cells, mRNA expression of genes SREBP-1, PPARγ, C/EBPα, LDLR, and adiponectin appeared to be significantly increased by 27.6%, 68.3%, 45.7%, 63.1%, and 45.5% respectively comparing to control group. Interestingly, 8 μM berberine treatment for 8 days post differentiation appeared to produce decreased mRNA expression of genes SREBP-1, PPARγ, C/EBPα, GLUT1, and adiponectin by 68.9%, 87.7%, 94.7%, 82.3%, and 96.3% respectively comparing to clozapine treated group. No significant mRNA expression change has been observed in FAS gene during clozapine treatment comparing to control group.

When treated with 50 μM risperidone for 8 days during differentiation of 3T3-L1 cells, mRNA expression of genes SREBP-1, PPARγ, C/EBPα, LDLR, Adiponectin, and FAS appeared to be increased by 133.5%, 103%, 48.8%, 61.5%, 63.8%, and 34.4% respectively comparing to control group. Interestingly, 8 μM berberine treatment for 8 days post differentiation appeared to produce decreased mRNA expression of genes SREBP-1, PPARγ, C/EBPα, LDLR, Adiponectin, and FAS by 80.9%, 93.1%, 96.4%, 90%, 98.2%, and 85.5% respectively, when compared to the risperidone treated group.

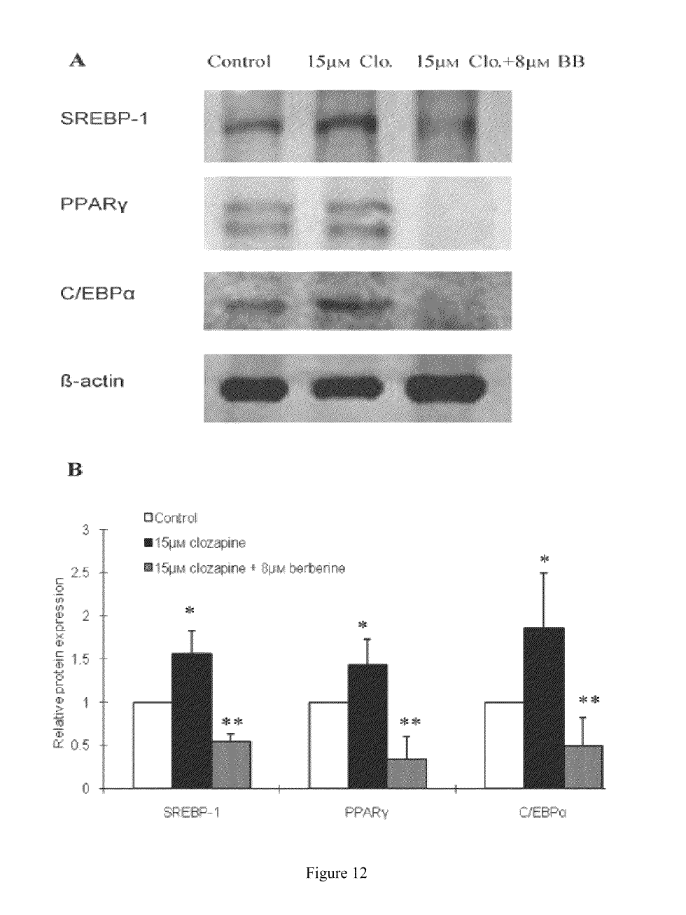

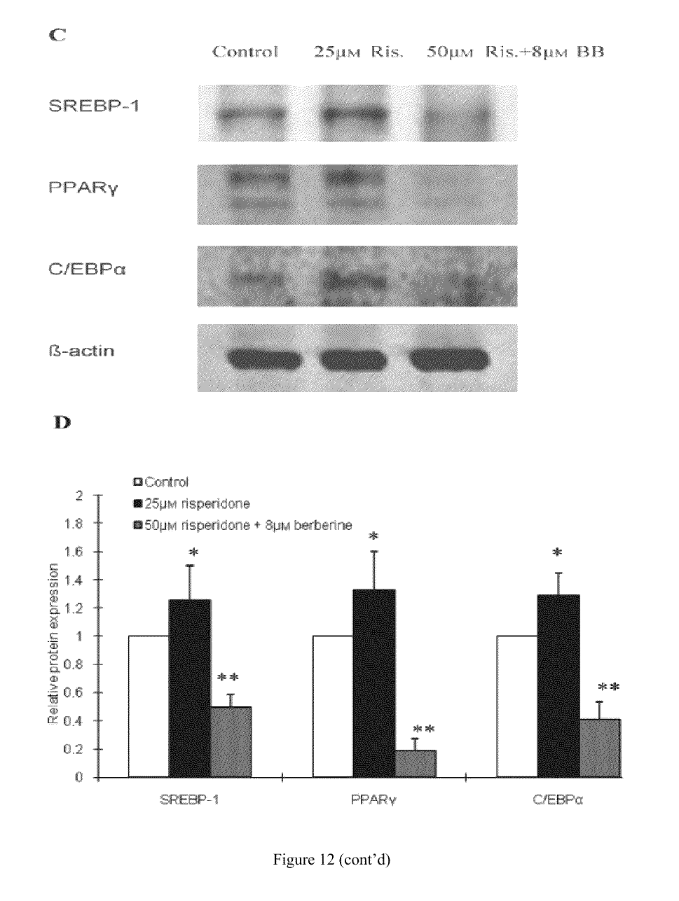

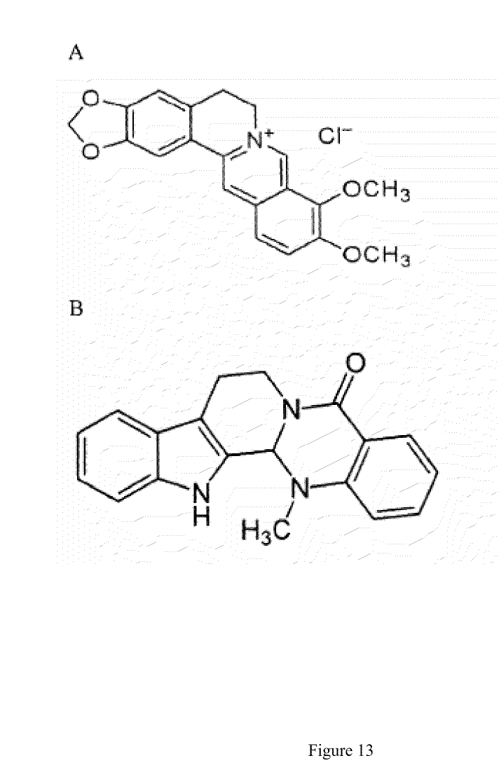

Immunoblotting: To further illustrate the mechanisms of inhibitory adipogenesis of berberine on 3T3-L1 induced by clozapine and risperdone, Western blot was used to test the protein expression of SREBP-1, PPARγ, C/EBPα during 3T3-L1 differentiation when treated with clozapine and berberine (FIG. 12A) or risperidone and berberine (FIG. 12C), and quantification of bands intensity relative to β-actin were calculated (FIG. 12B and FIG. 12D).

When treated with 15 μM clozapine for 8 days during differentiation of 3T3-L1 cells, protein expression of SREBP-1, PPARγ, C/EBPα relative to the protein expression of β-actin and normalized with controls appeared to be increased by 55.7%, 43.2%, 85.6% respectively comparing to control group. Following 8 μM berberine treatment for 8 days during differentiation, the protein expression of genes SREBP-1, PPARγ, C/EBPα relative to the protein expression of β-actin and normalized with controls appeared decreases by 64.6%, 76%, and 73% respectively comparing to clozapine treated group. The trends of protein expression appeared to be consistent with the finding of mRNA expression though the varied extents.

When treated with 50 μM risperidone for 8 days during differentiation of 3T3-L1 cells, protein expression of SREBP-1, PPARγ, C/EBPα relative to the protein expression of β-actin and normalized with controls appeared to be increased by 25.7%, 32.8%, and 28.9% respectively comparing to control group. Following 8 μM berberine treatment for 8 days during differentiation, the protein expression of SREBP-1, PPARγ, C/EBPα relative to the protein expression of β-actin and normalized with controls appeared to decrease by 60.4%, 85.7%, and 68% respectively comparing to risperidone treated group. The trends of protein expression were very consistent with the finding of mRNA expression though the varied extents.

### Discussion:

Atypical antipsychotics appear to have a higher affinity to the 5-HT2A receptor than to the D2 receptor and also appear to cause less extra pyramidal symptoms risks. Because of their broader spectrum and tolerable side effects, atypical antipsychotics, instead of the conventional antipsychotics, become the first line pharmacotherapy for schizophrenia. Unfortunately, atypical antipsychotics are strongly associated with weight gain. In a study over 10 weeks of clozapine and risperidone treatment the weight gain appeared to be approximately 4 kg and 2 kg respectively accompanied by increased triglyceride levels. The enhanced adipogenesis during preadipocyte differentiation has been postulated to one critical mechanism for such a side effect by previous studies.

In the present study, results illustrated that both 15 μM and 30 μM clozapine appeared to induce adipogenesis significantly. Interestingly, 30 μM clozapine appeared to induce less adipogenesis, which could be due to its cytotoxicity to 3T3-L1 cells as reflected by an MTT assay.

Pharmacological agents such as metformin, orlistat, and sibutramine are effective in general obesity but appear to have no statistically significant effect in antipsychotic-induced weight gain, which could be because they did not target adipocyte differentiation. Berberine has been reported to inhibit adipogenesis during differentiation in cells and in animal models by several research groups and recent clinical trial indicated that berberine reduced blood triglyceride level in type II diabetes patients. In this study, results indicated that the amount of adipogenesis appeared to be the same among control, 15 μM clozapine and 30 μM clozapine groups associated with 8 μM berberine treatment, which appeared to indicate berberine diminished adipogenesis completely. Similar results were observed in berberine treatment associated with risperidone induction. This appears to indicate that berberine is effective in preventing adipogenesis induced by atypical anti-psychotics in a cell model.

Sterol regulatory element-binding protein (SREBP)-1 is a key regulator of fatty acid metabolism and plays a pivotal role in the transcriptional regulation of different lipogenic genes mediating lipid synthesis. Enhanced expression of SREBP-1 in 3T3-L1 preadipocytes leads to a dramatic increase of its downstream genes and differentiation markers such as PPARγ, C/EBPα, LDLR, adiponectin, and FAS. In this study, SREBP-1 and related proteins were exclusively investigated during differentiation of 3T3-L1 with clozapine and risperidone treatments. The results indicated that treatment with 15 μM clozapine or 50 μM risperidone resulted in up-regulated mRNA expression of SREBP-1, PPARγ, C/EBPα, LDLR, Adiponectin, and similar protein expression were verified by western blot, which illustrate one possible mechanism of weight gain induced by clozapine and risperidone could be enhanced adipogenesis via SREBP-1 pathway.

Taken together, clozapine and risperidone appeared to induce adipogeneis in 3T3-L1 cells via SREBP-1 related pathway, which could explain the cause of their effect in human subjects. Furthermore, berberine appears to inhibit adipogenesis induced by clozapine and risperidone via SREBP-1 pathway in 3T3-L1 cells, which implies berberine could be a potential drug to prevent adipogenes induced by atypical antipsychotics.

TABLE 1
Primer Sequences for Example Two

| Oligonucleotide | Sequences (5′ to 3′) | SEQ ID NO |
|---|---|---|
| Mouse SREBP1-F | TATGGAGGGCATGAAACCCGAAGT | SEQ ID NO: 11 |
| Mouse SREBP1-R | TTGACCTGGCTATCCTCAAAGGCT | SEQ ID NO: 12 |
| Mouse PPARγ-F | TGGAATTAGATGACAGTGACTTGG | SEQ ID NO: 13 |
| Mouse PPARγ-R | CTCTGTGACGATCTGCCTGAG | SEQ ID NO: 14 |
| Mouse CEBPα-F | AGAAGTCGGTGGACAAGAACAGCA | SEQ ID NO: 15 |
| Mouse CEBPα-R | GCGTTGTTTGGCTTTATCTCGGCT | SEQ ID NO: 16 |
| Mouse LDLR-F | CAACAATGGTGGCTGTTCCCACAT | SEQ ID NO: 17 |
| Mouse LDLR-R | ACTCACACTTGTAGCTGCCTTCCA | SEQ ID NO: 18 |
| Mouse Adiponectin-F | AGACCTGGCCACTTTCTCCTCATT | SEQ ID NO: 19 |
| Mouse Adiponectin-R | AGAGGAACAGGAGAGCTTGCAACA | SEQ ID NO: 20 |
| Mouse FAS-F | GGTGTGGTGGGTTTGGTGAATTGT | SEQ ID NO: 21 |
| Mouse FAS-R | TCACGAGGTCATGCTTTAGCACCT | SEQ ID NO: 22 |
| Mouse β Actin-F | TGTGATGGTGGGAATGGGTCAGAA | SEQ ID NO: 23 |
| Mouse β Actin-R | TGTGGTGCCAGATCTTCTCCATGT | SEQ ID NO: 24 |

### Example 3

### Inhibitory Effects and Transcriptional Impact of Berberine and Evodiamine on Human White Preadipocyte Differentiation

### Materials and Methods

Cell culture: Cell culture was carried out following the protocol provided by Promocell Company. Briefly, human white preadipocytes (HWP's 32/female/caucasian) were cultured at 37° C. in a humidified 5% CO₂ atmosphere and grown in a preadipocyte Growth medium (GM) with 100 units/ml penicillin and 100 μg/ml streptomycin until confluence (day 0). Differentiation was induced with preadipocyte Differentiation Medium (DM) for 3 days (days 3), where upon the medium was changed to adipocyte Nutrition Medium (NM) and cultured for 12 days (days 15) (HWP cell line and media were purchased from Promocell, Heidelberg, Germany). Varying concentrations of berberine (Sigma-Aldrich, St. Louis, Mo., USA), evodiamine (Sigma-Aldrich, St. Louis, Mo., USA) and their combination were added to DM and NM in order to observe their effects.

MTT assay: To detect the effect of berberine, evodiamine and their combination on the viabilities of HWP during differentiation induction, HWP's were plated in 96-well culture plates at a density of 4×10³ cells/well and cultured in GM until confluent, then cultured in DM and NM supplemented with varying concentrations of berberine and evodiamine, alone and in combination. Medium was removed at different time points and MTT (0.5 mg/ml in NM, 50 Owen) was added. The plates were incubated at 37° C. for 4 hours, followed by the addition of DMSO (150 μl/well), and incubated at 37° C. for 1 hour. Optical density (OD) was measured at 570 nm with 650 nm as background.

Oil-Red-O Staining and quantification: Oil-Red-O Staining and quantitative Oil-Red-O Staining were performed as previously reported. Briefly, medium was removed and cells were washed with PBS twice, fixed with 3.7% formalin at room temperature for 30 min, then rinsed with water, added 60% 2-propanol and incubated for 5 minutes, then moved out 2-propanol and stained cells with Oil-Red-O solution (Oil-Red-O store solution (3 mg/ml in pure 2-propanol): water=3:2) at room temperature for 10 min. Then the Oil-Red-O solution was removed and cells were washed 3 times with water. Images were obtained using an Olympus IX70 inverted microscope equipped for phase-contrast microscopy (Olympus, Tokyo, Japan). After staining, the cells were washed twice with 70% ethanol to remove excess stain. Stained oil droplets in the cells were dissolved in 2-propanol containing 4% Nonidet-P40 (Fisher Scientific, Pittsburgh, Pa., USA) and OD values were measured at an absorbance of 490 nm.

Quantitative Real-Time RT-PCR: Real-Time reverse transcriptase polymerase chain reaction (RT-PCR) analysis was used to measure mRNA expression of human genes PPARγ, C/EBPα, GATA-2 and GATA-3 under the control of β-actin. Briefly, total RNA was isolated from HWP following treatment at days 15 with Trizol reagent (Invitrogen, Carlsbad, Calif., USA) according to the manufacturer's instruction. RNA was quantified using absorption of light at 260 and 280 nm, and sample integrity was checked by 1.5% agarose gel electrophoresis. 0.8 μg of total RNA from each sample was used for reverse transcription reaction using the TaqMan Reverse Transcription Reagents (Applied Biosystems, Foster City, Calif., USA) following the Manufacturer's instructions. PCR was done using the SYBR green Master Mix (Applied Biosystems, Foster City, Calif., USA) following the manufacturer's instructions. The primers (Integrated DNA Technologies, Coralville, Iowa, USA) sequences are shown in Table 1. The temperature cycling conditions of amplification were as follows: an initial step of denaturation at 95° C. for 10 min, 40 cycles of denaturation at 95° C. for 30 s, annealing at 55° C. for 30 s, and elongation at 72° C. for 30 s. Real-Time RT-PCR was performed using Mx3000P Real-Time Thermocyclers (Statagene, La Jolla, Calif., USA). The relative mRNA levels of these genes were calculated by Pfaffl's mathematical method (Pfaffl, M W, Nucleic Acid Res (2001) 29:e45) and normalized with control-treated groups.

Immunoblot Analysis: Protein expression of GATA-2, GATA-3, PPARγ and C/EBPα were assessed by Western Blot. Total proteins were isolated from HWP treated with compounds or vehicles on day 15. The cells were washed twice with PBS, lysed in a lysis buffer containing HEPES-Tx-PI buffer: 910 mM HEPES, 5 mM EDTA, 350 mM Sucrose, 1 μg/ml leupeptin, 1 μg/ml pepstatin, 1% Triton-X, 1 mM PMSF), processed with a 25G needle and centrifuged at 10,000 rpm for 5 min. Protein concentration was determined using the Micro BCA Protein Assay Kit (Fisher Scientific, Pittsburgh, Pa., USA) according to the manufacturers instructions. 20 μg proteins were loaded and separated by electrophoresis using the BioRad Electrophoretic System (60 V for 1 hour followed by 90 V for 5 hours). The proteins were then transferred to nitrocellulose membranes at 35 V overnight. The membrane was blocked for 1 hour at room temperature in 5% nonfat milk in Tris-buffered saline (TBS), then washed with Tween-20-TBS (TTBS, 0.1% Tween-20) three times (15 min, 5 min, 5 min), followed by incubation with primary antibodies human GATA-2, GATA-3, PPARγ, C/EBPα and β-actin (dilution 1:200) (all antibodies were purchased from Santa Cruz Biotechnology, Santa Cruz, Calif., USA) at room temperature for 2 hours. The membrane was rinsed three times (15 min, 5 min, 5 min), then incubated at room temperature for 1 hour with Horseradish peroxidase-conjugated second antibody (1:10,000). The membrane was then incubated in ECL reagent (GE Healthcare, Piscataway, N.J., USA) for 1 minute. Protein was visualized using the UVP image analysis system (UVP, Upland, Calif., USA).

Statistical analysis: The data was analyzed by the ANOVA procedure of Statistical Analysis System (SAS Institute, 1999-2001). Significant differences between groups were determined using Student's t-test or Duncan's multiple range tests at the p<0.05 (*).

### Results:

A. Berberine does not appear to affect Human White Preadipocyte (HWP) viability whilst evodiamine decreases HWP viability. Berberine appears to reverse the viability inhibition effect of evodiamine when used in combination.

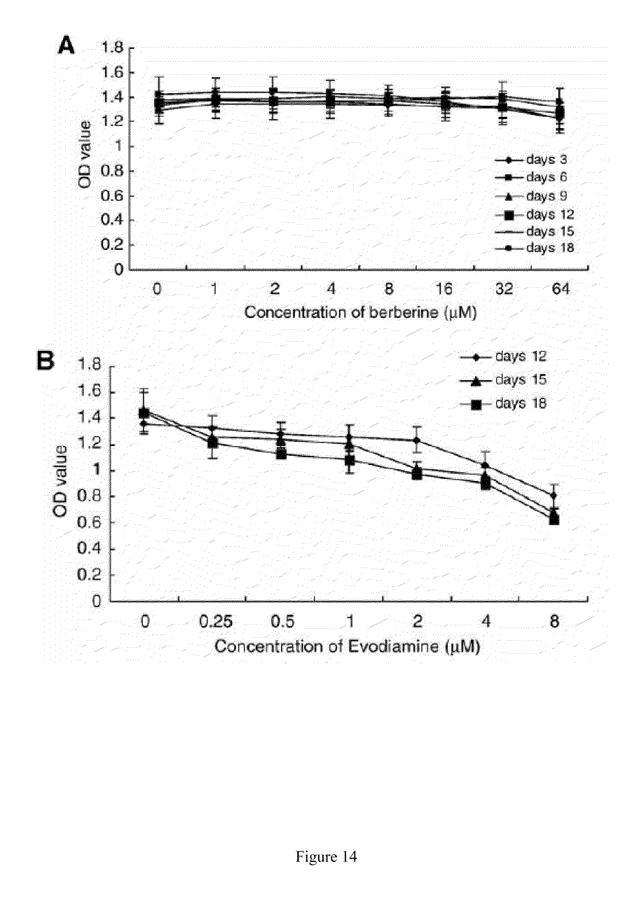

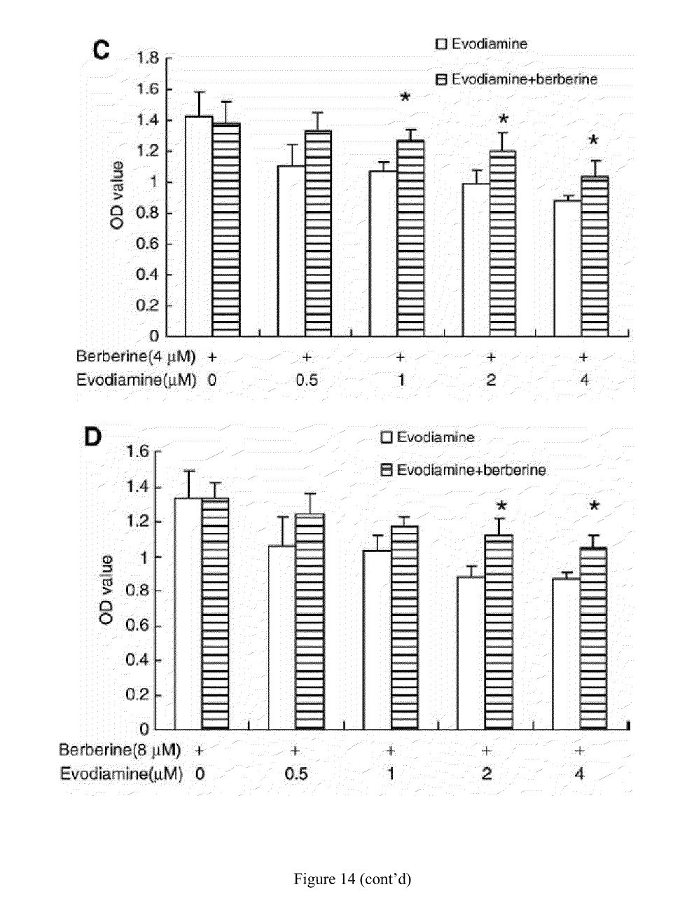

MTT analysis was carried out at days 3, 6, 9, 12, 15, and 18 during differentiation to detect the effect of berberine on the viability of HWP (FIG. 14A). HWP were treated with various concentration of berberine (0, 1, 2, 4, 8, 16, 32, 64 μM) during each stage. Berberine showed no significant effects on the HWP viability. For example, after inducing differentiation for 15 days, 1, 2, 4, and 8 μM berberine decreased cell viability by −3.1%, 1.1%, −0.0% and 1.3% respectively compared to controls.

MTT analysis was carried out at days 12, 15, and 18 during differentiation to detect the effect of evodiamine on the viability of HWP (FIG. 14B). HWP were treated with various concentrations of evodiamine (0, 0.25, 0.5, 1, 2, 4, 8 μM) during each stage. Evodiamine appeared to decrease the HWP viability significantly. For example, after inducing differentiation for 15 days, 0.25, 0.5, 1, 2, 4, and 8 μM evodiamine appeared to decrease cell viability by 14.1%, 15%, 17.7%, 30.5%, 34.2% and 53.6% respectively when compared to the control group.

MTT analysis was carried out at days 15 during differentiation to detect the effect of the combination of berberine and evidiamine on the viability of HWP (FIGS. 14C and 14D). HWP were treated with various concentrations of berberine (4, 8 μM), evodiamine (0.5, 1, 2, 4 μM) and their combination (4 μM berberine+0.5, 1, 2, 4 μM evodiamine, or 8 μM berberine+0.5, 1, 2, 4 μM evodiamine). The combinations of 4 μM berberine with 1, 2, and 4 μM evodiamine appeared to compensate the viability inhibition of 1, 2, and 4 μM evodiamine on HWP while 4 μM berberine alone appeared to have the lowest inhibition of cell viability. The combinations of 8 μM berberine with 2 and 4 μM evodiamine appeared to compensate for the viability inhibition of 2, 4 μM evodiamine on HWP while 8 μM berberine alone appeared to have the lowest inhibition of cell viability.

### B. Berberine and Evodiamine Appear to Inhibit Differentiation of Human White Preadipocyte (HWP) Individually and in Combination.

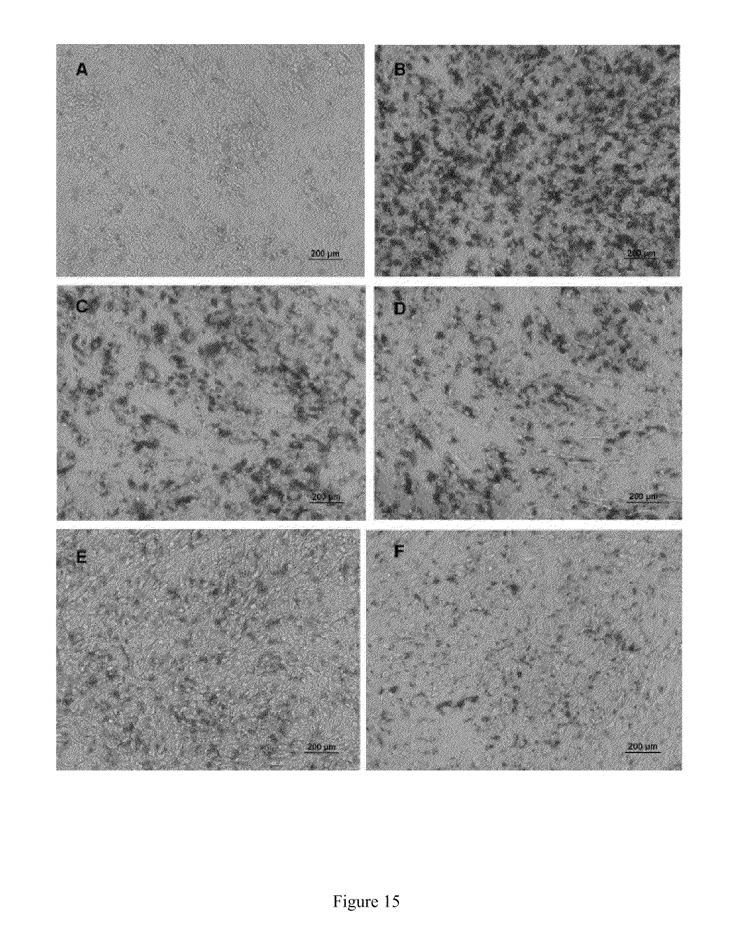

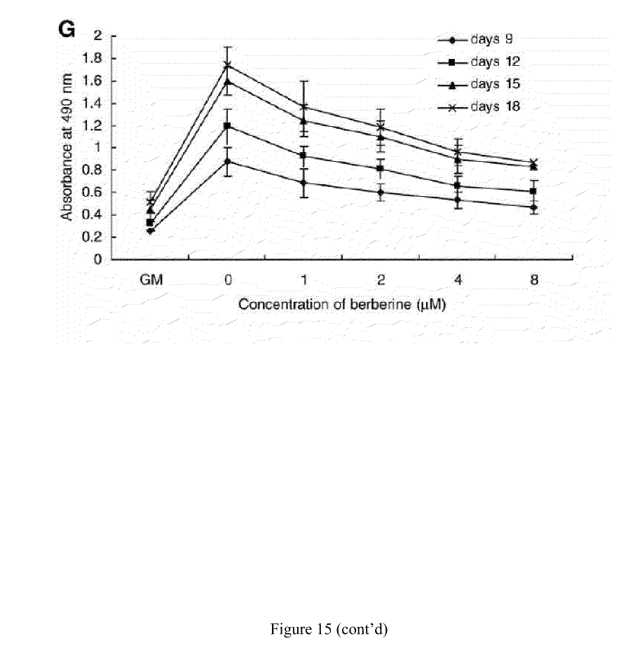

In order to study the potential inhibitory effects of berberine on HWP differentiation, various concentrations of berberine (0, 1, 2, 4, 8 μM) were added to the Differentiation Medium (DM) and Nutrition Medium (NM) during the culture of HWP. Differentiation was monitored as previously described by Oil-Red-O (FIG. 15) and shows the staining of HWP 15 days post-differentiation and the quantitative results of HWP at days 9, 12, 15 and 18 post differentiation. As expected, in HWP cells cultured in GM alone there are very few red-stained cells (FIG. 15A), while also as expected the staining of HWP cultured in DM and NM revealed almost total differentiation (FIG. 15B). However, cells grown in DM and NM with the addition of 1 μM (FIG. 15C) and 2 μM (FIG. 15D) berberine, respectively, showed apparent inhibition of cellular differentiation. What is more, cells grown in the presence of 4 μM (FIG. 15E) and 8 μM (FIG. 15F) berberine appeared to display the maximum suppression of differentiation. Quantitative Oil-Red-O staining measurements (FIG. 15G) indicated that HWP grown in the presence of 1, 2, 4, or 8 μM berberine and 15 days post differentiation had a reduction in lipid content of 31.9%, 42.9%, 60.6% and 66.4% respectively when compared with the control-treated group (GM group).

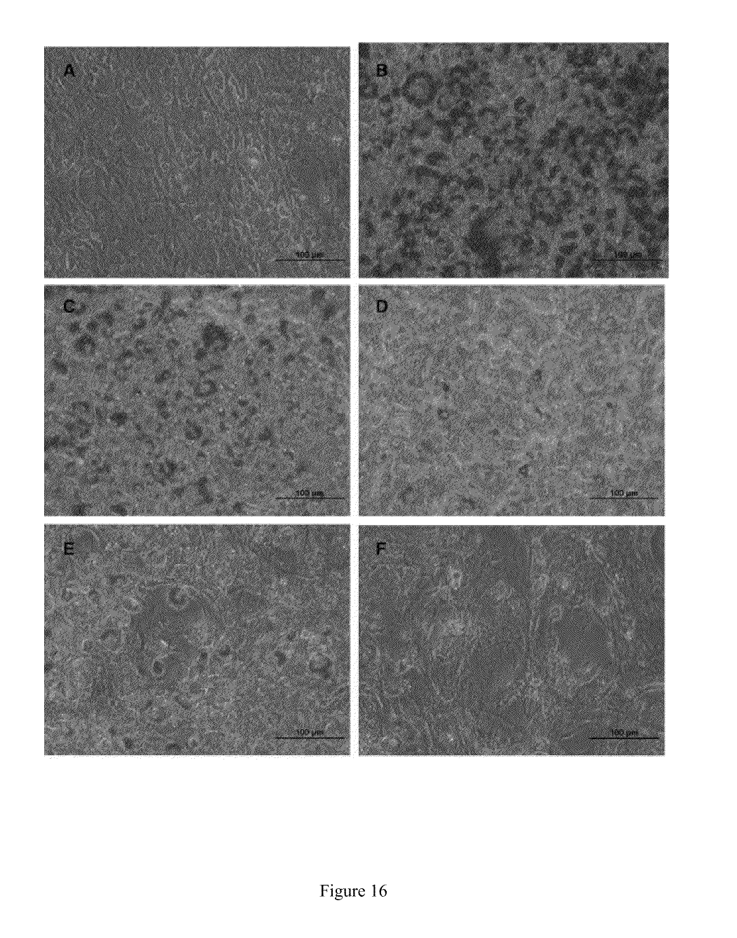

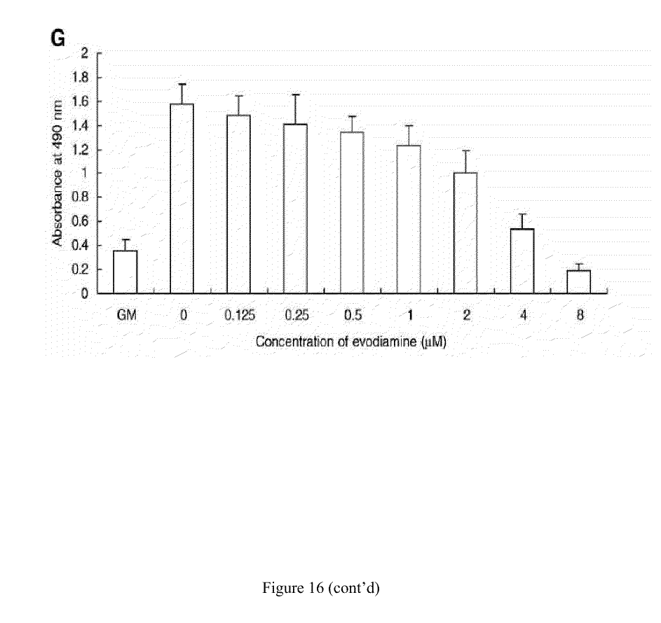

In order to study the potential inhibitory effects of evodiamine on HWP differentiation, various concentrations of evodiamine were added to DM and NM during HWP culture. The differentiation process was monitored as above. FIG. 4 illustrates the staining and quantitative results of HWP treated with evodiamine or vehicle 15 days post differentiation. As expected, the differentiation of HWP cultured in GM alone was negligible (FIG. 16A) and, again as expected, HWP cultured in DM and NM appeared to demonstrate almost total differentiation. However, cells grown in DM and NM with the addition of evodiamine at 0.5 μM (FIG. 16C), 1 μM (FIG. 16D) 2 μM (FIG. 16E), and 4 μM (FIG. 16F) indicated significant inhibition of cellular differentiation. Quantitative Oil-Red-O staining measurements (FIG. 16G) revealed that HWP treated with 0.125, 0.25, 0.5, 1, 2, 4, or 8 μM evodiamine 15 days post differentiation induction, had a reduction in lipid content of 7.5%, 13.8%, 19%, 27.8%, 46.7%, 84.6%, and 112.6% respectively when compared with the vehicle-treated group deduced with background (GM group).

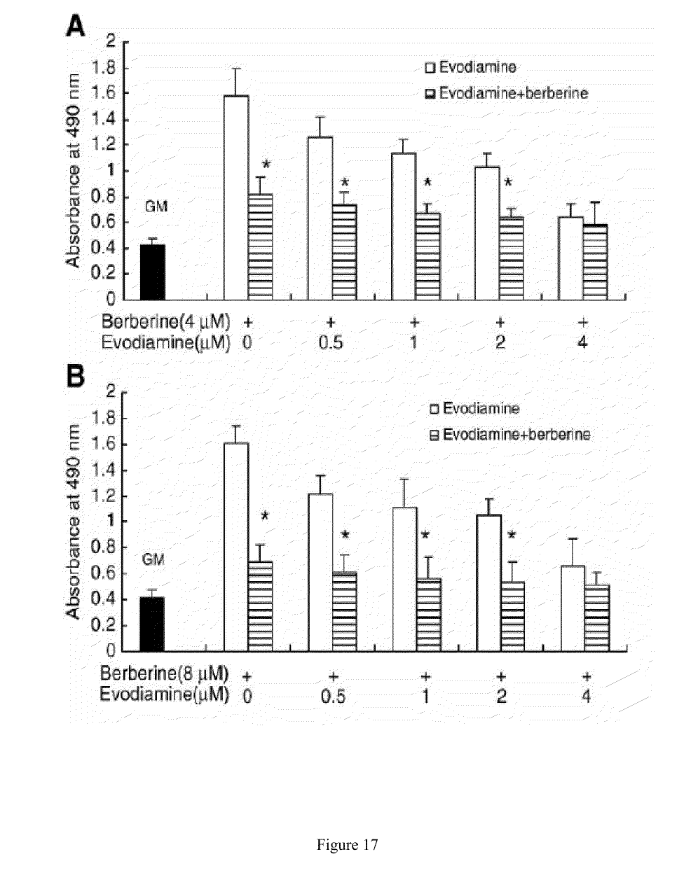

Apparent inhibitory effects of individual treatment with berberine and evodiamine were observed and the potential effect of a combination of both compounds on HWP differentiation was then analyzed. Thus, varying concentrations of berberine (4, 8 μM) and evodiamine (0.5, 1, 2, 4 μM) were added to DM and NM during HWP culture. FIG. 17 illustrates the quantitative results of Oil-Red-O staining on HWP 15 days post differentiation. HWP cultured in 0.5, 1, 2, or 4 μM evodiamine in combination with either 4 μM (FIG. 17A) or 8 μM berberine (FIG. 17B) appeared to exhibit less adipogenesis when compared to cells treated with evodiamine alone, however appeared to demonstrate similar adipogenesis when compared to cells treated with berberine alone.

C. Berberine and Evodiamine Appears to Increase the mRNA Expression of GATA-2 and GATA-3 Genes During the Inhibition of Human White Preadipocyte (HWP) Differentiation.

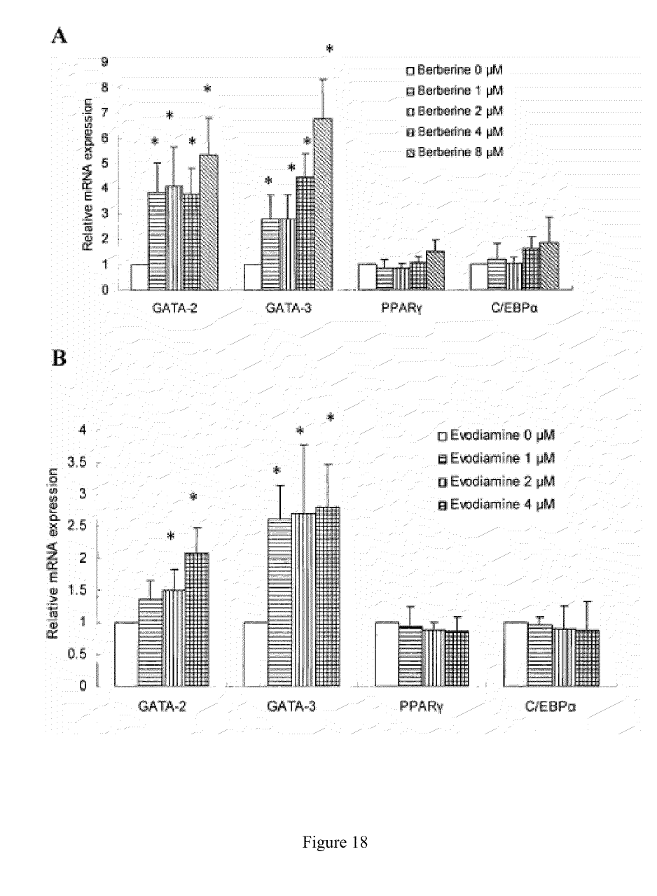

GATA-2 and GATA-3 mRNA expression appeared to be increased in cells cultured in DM and NM when treated with varying concentrations of berberine compared to cells cultured in media treated with vehicle (FIG. 18A). GATA-2 mRNA expression was increased to 3.87 fold (1 μM), 4.1 fold (2 μM), 3.8 fold (4 μM), and 5.33 fold (8 μM) comparing to vehicle (control) group. GATA-3 mRNA expression was increased to 2.77 fold (1 μM), 2.79 fold (2 μM), 4.43 fold (4 μM), and 6.77 fold (8 μM) when compared to the control group.

In addition, GATA-2 and GATA-3 mRNA expression also appeared increased in HWP cells cultured in DM and NM treated with varying concentrations of evodiamine (0, 1, 2, 4 μM) (FIG. 18B). GATA-2 mRNA expression also appeared to be increased 1.36 fold (1 μM), 1.5 fold (2 μM), and 2.08 fold (4 μM), whilst GATA-3 mRNA expression appeared to be increased 2.61 fold (1 μM), 2.7 fold (2 μM), and 2.79 fold (4 μM).

The data indicated that there appeared to be no significant changes in PPARγ and C/EBPα mRNA expression following treatment with berberine or evodiamine.

### D. Berberine and Evodiamine Appears to Increase GATA-2 and GATA-3 Protein Expression.

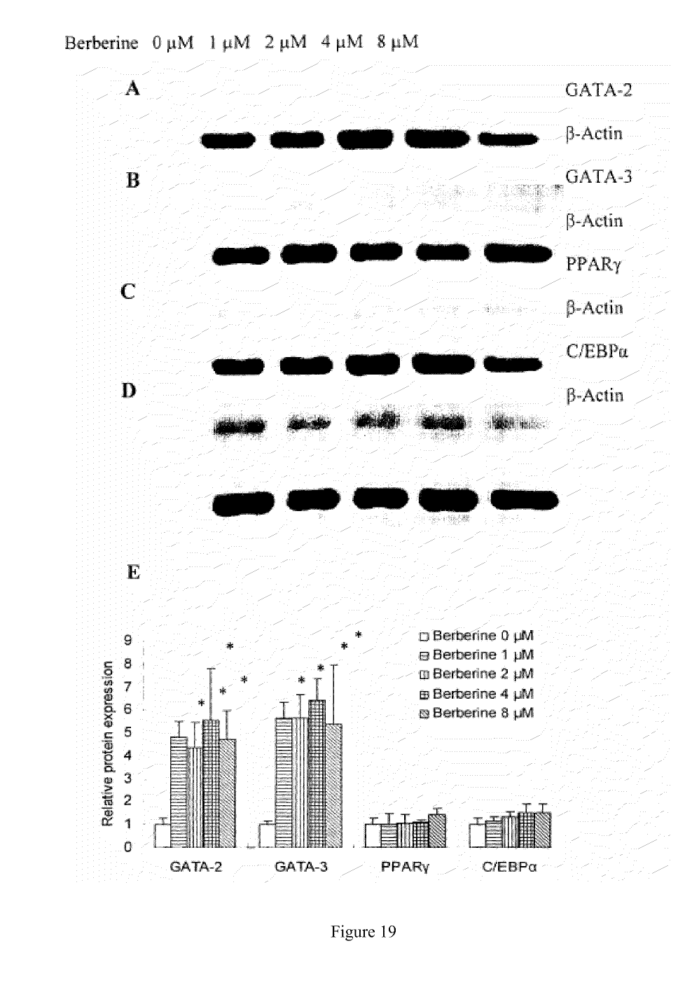

GATA-2 protein expression appeared increased in cells cultured in DM and NM containing various concentrations of berberine (FIG. 19A). Protein expression results were consistent with our GATA-2 mRNA expression. GATA-2 protein expression relative to the protein expression of β-actin and normalized with controls was increased to 4.8 fold (1 μM), 4.38 fold (2 μM), 5.53 fold (4 μM) and 4.72 fold (8 μM)(FIG. 19E).

GATA-3 protein expression also appeared increased in cells cultured in DM and NM containing berberine (FIG. 19B). Protein expression results were also consistent with mRNA expression findings. GATA-3 protein expression appeared to be increased to 5.63 fold (1 μM), 5.65 fold (2 μM), 6.43 fold (4 μM) and 5.37 fold (8 μM) (FIG. 19E).

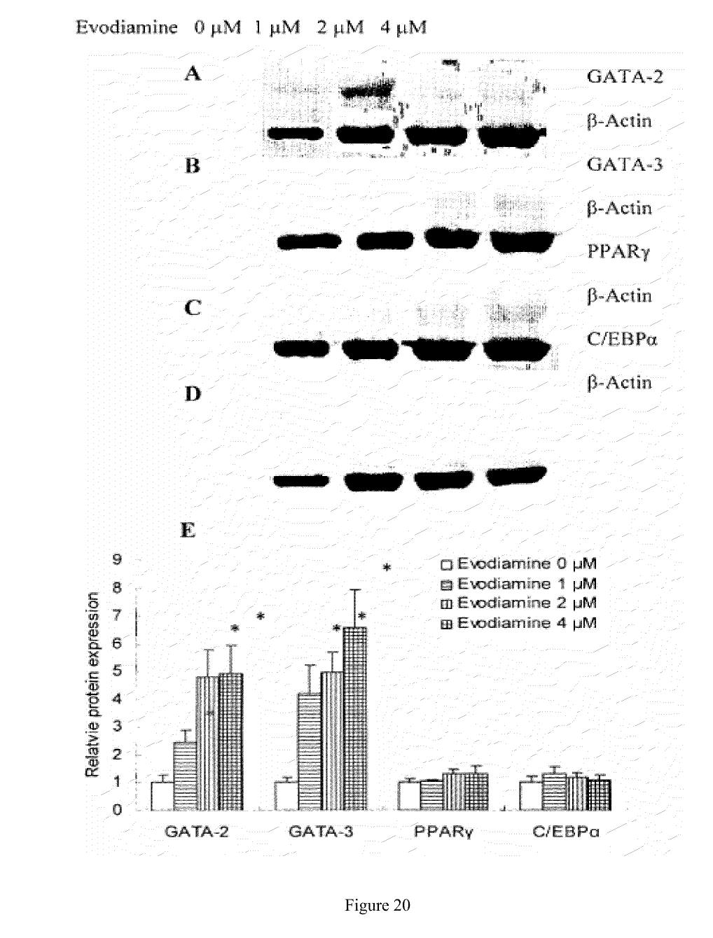

Evodiamine also appeared to increase the protein expression of GATA-2 and GATA-3. When treated with various concentrations of evodiamine (0, 1, 2, 4 μM), the GATA-2 and GATA-3 protein expression increased which was consistent with mRNA expression findings (FIG. 20A, B). GATA-2 protein expression was increased to 2.44 fold (1 μM), 4.81 fold (2 μM), and 4.92 fold (4 μM). Whilst GATA-3 protein increased 4.21 fold (1 μM), 4.95 fold (2 μM), and 6.57 fold (4 μM) when compared to controls (FIG. 20E).

Consistent with the finding of mRNA expression, the PPARγ and C/EBPα protein expression appeared to have no significant difference between drug-treated cells and control cells for either berberine (FIGS. 19C, 19D and 19E) or evodiamine (FIGS. 20C, 20D and 20E).

### Discussion:

Adipocyte differentiation appears to play a crucial role in obesity and the process of adipogenesis has recently become a major target of obesity research. It is estimated that the structural and functional morphogenesis associated with adipocyte differentiation involves changes in the expression levels of approximately 300 proteins, among those are the master adipogenic regulators—transcription factors C/EBPα and PPARγ. Recent research has reported that the GATA family of transcriptional regulators plays a key role in adipogenesis.

Two isoforms, GATA-2 and GATA-3, are specifically expressed in murine preadipocyte but not mature adipocytes and continuous expression of GATA-2 and 3 in preadipocytes cell lines appears to inhibit terminal differentiation into mature adipocytes. Berberine and evodiamine are alkaloids isolated from many Chinese herbs have been demonstrated to have many pharmacological effects including decreasing PPARγ and C/EBPα expression during mouse preadipocyte 3T3-L1 differentiation. However, there have been no reports of the effects of berberine or evodiamine on Human white preadipocyte differentiation.

In the present study, it appears that berberine has no inhibitory effect on the viability of HWP during differentiation which is consistent with previous studies in 3T3-L1 cells. Evodiamine, however, appears to decrease the cell viability predominantly in HWP (8 μM decreased cell viability by 53.6%), which was not observed in the previous mouse 3T3-L1 study. This study shows that the combination of berberine and evodiamine inhibits viability significantly less than evodiamine alone which could be advantageous with regards to a future potential in anti-obesity drug development.

Importantly, this report demonstrates that both berberine and evodiamine appear to inhibit adipogenic differentiation in HWP. However, an apparent greater inhibition of adipogenesis when cells were treated with a combination of berberine and evodiamine than with berberine alone was not observed, where berberine alone had the highest inhibition quantitatively.

In addition, the present study also appeared to demonstrate an important inhibitory effect on gene and protein expression in HWP. Berberine and evodiamine appeared to increase expression of GATA-2 and 3 both at the gene and protein level. Interestingly, there did not appear to be differences in either gene or protein expression of PPARγ and C/EBPα which may be a result of cell line specific gene expression.

In light of these results, it appears that an understanding of the molecular mechanisms and signaling pathways by which berberine and evodiamine interact in the complex gene expression patterns involved in adipocyte differentiation will be critically important if berberine and evodiamine are to be used as future anti-obesity agents. It also appears that berberine and evodiamine have potential as anti-obesity agents.

### Example 4

### Berberine Appears to Inhibit Adipogenesis in High-Fat Diet-Induced Obesity Mice

### Materials and Methods

Mouse experiments: All experiments were approved by Institutional Animal Care and Use Committee of South Dakota State University. Nine-week-old C57BL/6J male mice were purchased from The Jackson Laboratory. Group 1 mice were fed with normal diet (ND, D12450Bi, Research Diets Inc. New Brunswick, N.J., USA) for 9 weeks, group 2 mice were fed with ND for 6 weeks and then high-fat diet (FD, D12451i, Research Diets Inc. New Brunswick, N.J., USA) for 3 weeks. The mice were maintained according to The Jackson Laboratory guidelines for animal care and housed at 22±2° C. temperature, 55±5% relative humidity, with a light/dark cycle of 12 hours. Food (group 1 with normal fat diet and group 2 with high-fat diet until sacrifice) and water were provided ad libitum. After stabilizing for 2 weeks and balancing the difference in body weight between the subgroups, the mice were intraperitoneally injected with various concentrations of berberine (Sigma-Aldrich, Saint Louis, Mo., USA) or vehicle (PBS) solution for 36 days (Group 1, subgroup 1: normal diet mice treated with vehicle, subgroup 2: normal diet mice treated with 3 mg/kg/day berberine. In group 2, subgroup 1: high-fat diet mice treated with vehicle, subgroup 2-4: high-fat diet mice treated with 0.75, 1.5, 3 mg/kg/day berberine respectively). Six mice per subgroup were obtained finally. Mouse weight and food intake were recorded every 3 days with the day of treatment with berberine or control as day 0. Blood samples were collected from the hearts of anesthetized mice after feeding and treatment for 36 days following fast for 16 hours. When sacrificed, mouse liver, kidney, spleen and epididymal fat tissues were dissected and weighed. Epididymal fat tissues were immediately frozen in liquid nitrogen and stored at −80° C.

Serum glucose, triglyceride, cholesterol assay: Blood samples from each mouse were collected and serum was separated by centrifugation for the measurement of levels of glucose, triglyceride and total cholesterol. Serum glucose concentrations were analyzed using Glucose (HK) Assay Kit (Sigma-aldrich, Saint Louis, Mo., USA). Serum triglyceride concentrations were analyzed using Serum Triglyceride Determination Kit (Sigma-aldrich, Saint Louis, Mo., USA). Serum cholesterol concentrations were analyzed using Amplex® Red Cholesterol Assay Kit (Invitrogen, Carlsbad, Calif., USA). All experimental assays were done according to the manufacturer's instructions.

RNA and protein purification and quantification from epididymal fat tissues: RNA and protein was isolated from 60 mg of epididymal fat tissues with Trizol (Invitrogen, Carlsbad, Calif., USA) following the protocol recommended by the manufacturers. RNA was dissolved in pure water and quantified at 260/280 nm, and sample integrity was checked by 1.5% agarose gel electrophoresis. Protein concentration was determined using the Micro BCA Protein Assay Kit (Fisher Scientific, Pittsburgh, Pa., USA) according to the manufacturer's instructions.

Quantitative Real-Time RT-PCR: Real-Time reverse transcriptase polymerase chain reaction (RT-PCR) analysis was used to measure mRNA expression of genes PPARγ, C/EBPα, GATA-2 and GATA-3 in the control of β-actin. Total RNA (0.8 μg) from each sample was used for a reverse transcription reaction using the TaqMan Reverse Transcription Reagents (Applied Biosystems, Foster City, Calif., USA) following the manufacturer's instructions. PCR was done using the SYBR green Master Mix (Applied Biosystems, Foster City, Calif., USA) following the manufacturer's instructions. The primers (Integrated DNA Technologies, Coralville, Iowa, USA) sequences are shown in Table 1. The cycling conditions of amplification were as follows: an initial step of denaturation at 95° C. for 10 min, 40 cycles of denaturation at 95° C. for 30 s, annealing at 55° C. for 30 s, and elongation at 72° C. for 30 s. Real-Time PCR was performed using Mx3000P Real-Time Thermocyclers (Stratagene, La Jolla, Calif., USA). The relative mRNA levels of these genes were calculated by 2-delta delta CT method and normalized with control-treated groups.

Immunoblot Analysis: Protein expression of GATA-2, GATA-3, PPARγ and C/EBPα in the control of β-actin was assessed by Western Blot. Protein samples from the same group were homogenized as previously reported (Hu and Davies, Phytomedicine (2009) 16:864-873). Protein (25 μg) was separated by polyacrylamide gel electrophoresis using the BioRad Electrophoretic System (60 V for 1 h followed by 90 V for 5 h). The proteins were then transferred to nitrocellulose membranes at 35 V overnight. The membrane was blocked for 1 h at room temperature in 5% nonfat milk in Tris-buffered saline (TBS), then washed with Tween-20-TBS (TTBS, 0.1% Tween-20) three times (15 min, 5 min, 5 min), followed by incubation with primary antibodies mouse GATA-2, GATA-3 (dilution 1:200), PPARγ, C/EBPα and β-actin (dilution 1:500) at room temperature for 2 h (all antibodies were purchased from Santa Cruz Biotechnology, Santa Cruz, Calif., USA). The membrane was rinsed three times (15 min, 5 min, 5 min), then incubated at room temperature for 1 h with horseradish peroxidase-conjugated second antibody (1:10,000). The membrane was then incubated in ECL reagent (GE Healthcare, Piscataway, N.J., USA) for 1 min. Protein was visualized using the UVP image analysis system (UVP, Upland, Calif., USA).

Statistical analysis: The data were analyzed by the ANOVA procedure of Statistical Analysis System (SAS Institute, 1999-2001). Significant differences between groups were determined using Student's t-test or Duncan's multiple range tests at the p<0.05 (*).

### Results:

A. Berberine Appeared to Reduce Weight Gain and Food Intake in FD Mice while Having No Effects on ND Mice.

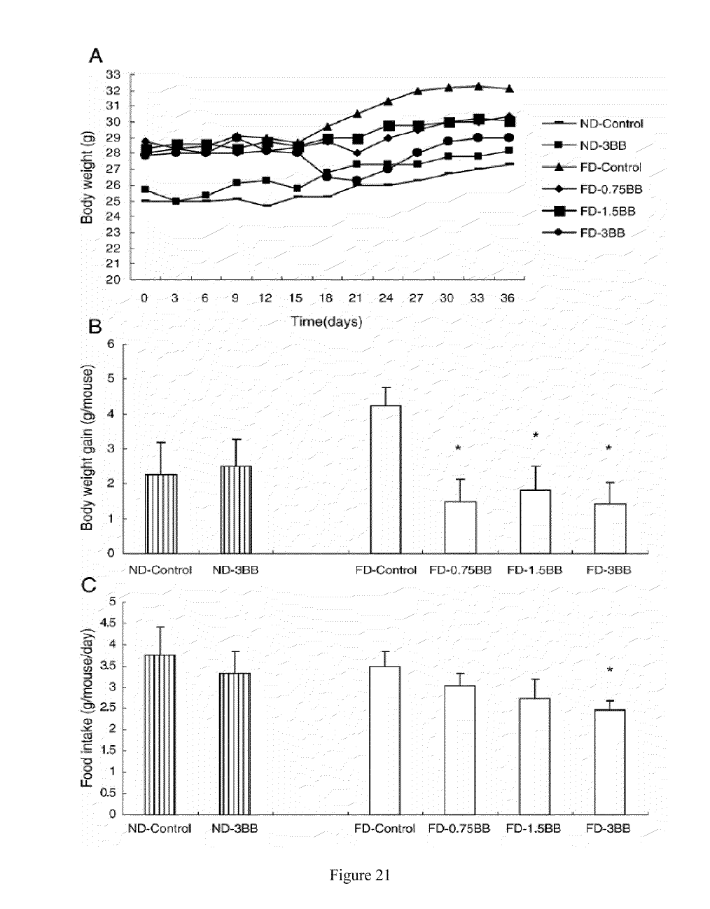

Throughout the 36 days of study of treatment either with berberine or control, the body weight of each mouse was monitored every 3 days (FIG. 21A). At the beginning of treatment, 11-week-old FD mice had higher initial weight than ND mice (28.3 g/mouse comparing to 25.4 g/mouse). When comparing mouse weight from treatment initiation to date of sacrifice (FIG. 21B), berberine appeared to decrease the weight gain significantly in the FD mice group (weight gain in control treated subgroup was 4.3±0.6 g/mouse, whilst in mice treated with 0.75, 1.5, or 3 mg/kg/day berberine, mouse weights were 1.5±0.7 g/mouse, 1.9±0.7 g/mouse, 1.4±0.5 g/mouse respectively). However, in the ND mouse group, 3 mg/kg/day berberine did not appear to affect the weight gain significantly (2.3±0.9 g/mouse in the control treated group compared to 2.5±0.7 g/mouse in 3 mg/kg/day berberine treated group). When fed with FD (FIG. 21C), mice treated with berberine consumed a lower amount of food (food intake in control treated group was 3.5±0.4 g/mouse/day, however, in mice treated with 0.75, 1.5, 3 mg/kg/day berberine it was 3.0±0.3 g/mouse/day, 2.7±0.5 g/mouse/day, 2.5±0.2 g/mouse/day respectively) with the group treated with 3 mg/kg/day berberine demonstrating significantly lower food intake as compared to the control treated group. In the ND mice group, 3 mg/kg/day berberine decreased the food intake amount slightly but not significantly (3.8±0.7 g/mouse in control treated subgroup and 3.3±0.5 g/mouse/day in 3 mg/kg/day berberine treated group).

B. Berberine appeared to reduce the weight of epididymal fat and liver relative to body weight, But Did not Affect the Weight of Kidney or Spleen in FD Mice and Had No Effect on ND Mice.

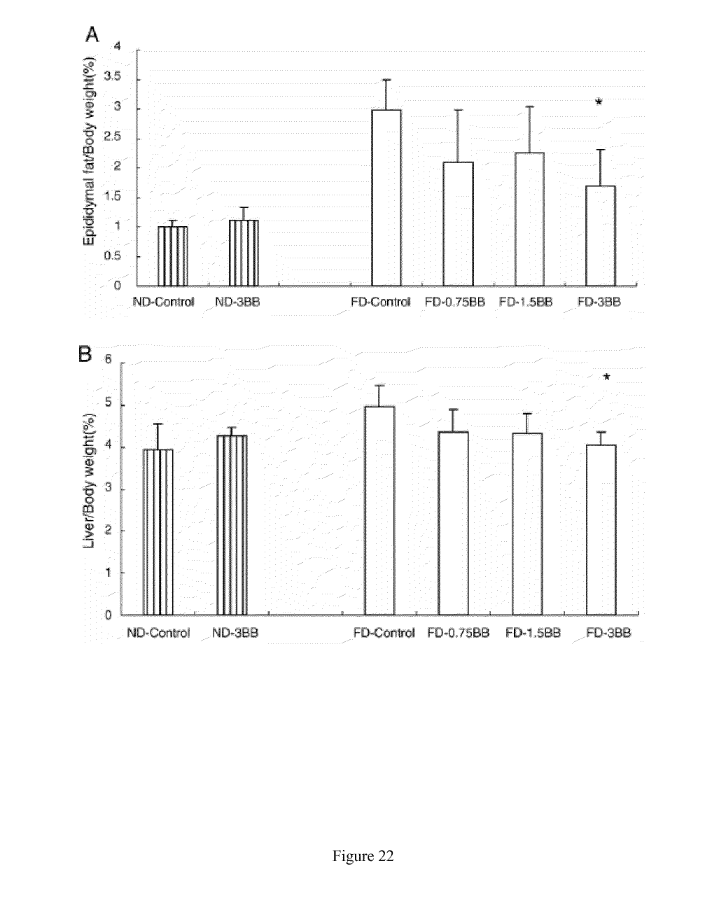

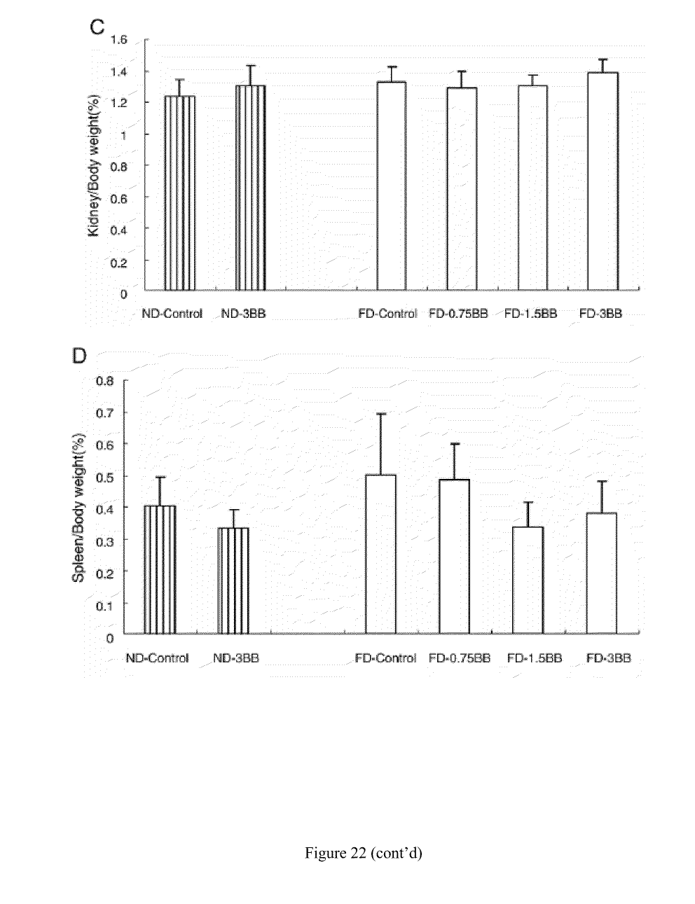

To examine if body weight reduction was due to a decrease in adipogenesis, mice were sacrificed and epididymal fat pads were dissected and weighed (FIG. 22A). The average weight of epididymal fat relative to body weight in the FD mice group appeared to be much higher than in the ND mice group (2.3±0.6% in the FD group compared to 1.1±0.1% in ND group). In the FD group, the weight of epididymal fat relative to body weight was decreased in the berberine treated mice (control treated group was 3.0±0.5%, 0.75, 1.5, 3 mg/kg/day whilst the berberine treated group were 2.1±0.9%, 2.3±0.8%, 1.7±0.6% respectively). Berberine appeared to decrease the fat ratio by 29.7±7.1%, 23.9±7.5%, 43.6±7.9% compared to the control treated subgroup, and 3 mg/kg/day berberine appeared to demonstrate significant effects as compared to the control treated subgroup. In the ND mice group, berberine appeared to decrease the fat ratio slightly but not significantly effect the weight of epididymal fat relative to body weight (1.0±0.1% in the control treated subgroup and 1.13±0.2% in the 3 mg/kg/day berberine treated subgroup).

Liver weight relative to body weight in the ND mice group and FD mice group was 4.1% and 4.4% respectively. In the FD mice group, 3 mg/kg/day berberine decreased the liver ratio significantly (5.0±0.5% in vehicle treated subgroup and 4.0±0.4% in 3 mg/kg/day berberine treated subgroup). There was no significant difference in liver weight between the control and berberine treated subgroups in the ND mice group (FIG. 22B). There was no significant difference in kidney weight relative to body weight among the FD mice subgroups (control subgroup: 1.3±0.1%, 0.75, 1.5, 3 mg/kg/day berberine subgroups were 1.3±0.1%, 1.3±0.1%, 1.4±0.1% respectively) and the ND mice subgroups (control subgroup: 1.2±0.1%, 3 mg/kg/day berberine subgroup: 1.3±0.1%) (FIG. 22C). There was no significant difference in spleen weight relative to body weight among FD mice subgroups (control subgroup: 0.5±0.2%, 0.75, 1.5, 3 mg/kg/day berberine subgroups were 0.5±0.1%, 0.3±0.1%, 0.4±0.1% respectively) and ND mice subgroups (control subgroup: 0.4±0.1%, 3 mg/kg/day berberine subgroup: 0.3±0.1%) (FIG. 22D).

### C. Berberine Appeared to Reduce the Serum Concentrations of Glucose, Triglyceride and Total Cholesterol in FD Mice.

Serum samples were obtained from each mouse to examine whether alterations in adiposity by berberine administration were also correlated with changes in serum glucose and lipid levels.

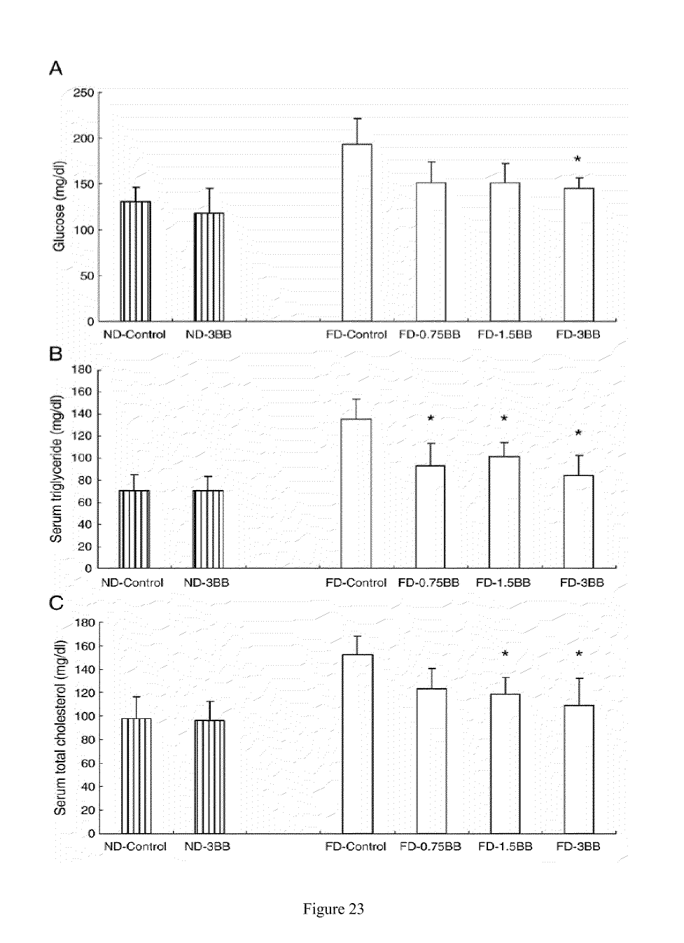

As shown in FIG. 23A, Berberine administration appeared to result in a significant reduction in fasting serum glucose levels in subgroups of FD mice (control subgroup: 195.4±27.3 mg/dl, 0.75, 1.5, 3 mg/kg/day berberine groups were 157.3±22.5 mg/dl, 151.8±21.1 mg/dl, 143.5±12.7 mg/dl respectively, 0.75, 1.5, 3 mg/kg/day berberine reduced the serum glucose level by 19.5%, 22.3% and 26.5% respectively when compared with the control treated subgroup). There did not appear to be a significant difference between 3 mg/kg/day berberine (130.9±16.2 mg/dl) and control (120.6±27.5 mg/dl) treated subgroups of ND mice.

As shown in FIG. 23B, in groups of FD mice, 36 days administration of berberine appeared to reduce the triglyceride levels significantly, (control group: 135.6±17.0 mg/dl, 0.75, 1.5, 3 mg/kg/day whilst the berberine group values were 93.2±20.8 mg/dl, 101.4±9.7 mg/dl, 84.3±18 mg/dl respectively). Thus, concentrations of 0.75, 1.5, 3 mg/kg/day berberine treatment appeared to reduce the serum triglyceride level by 31.2%, 25.2% and 37.8% respectively when compared with the control treated group. In the ND group, there appeared to be no significant difference in the triglyceride levels between 3 mg/kg/day berberine (70.7±12.8 mg/dl) and the control (70.7±14 mg/dl) treated groups. Berberine also appeared to significantly reduce total cholesterol (FIG. 23C) in FD mice following 36 days administration (control group: 152.4±15.2 mg/dl, 0.75, 1.5, 3 mg/kg/day whist the berberine levels were 123.9±16.5 mg/dl, 118.5±14.7 mg/dl, 109.8±22.7 mg/dl respectively). Thus, 0.75, 1.5, 3 mg/kg/day berberine reduced the serum total cholesterol level by 18.7%, 22.2% and 28% respectively. In the ND group, there appeared to be no significant difference in total cholesterol level between 3 mg/kg/day berberine (97.4±18.3 mg/dl) and the control (96.5±16.5 mg/dl) treated groups. Therefore, these data indicate that berberine may suppress the development of adiposity via the regulation of the serum glucose and lipid levels.

D. Berberine Appeared to Increase the mRNA Expression of Key Adipogenic Transcription Factors GATA-2, and GATA-3 Whilst Decreasing Expression of PPARγ and C/EBPα mRNA in the Epididymal Fat of FD Mice.

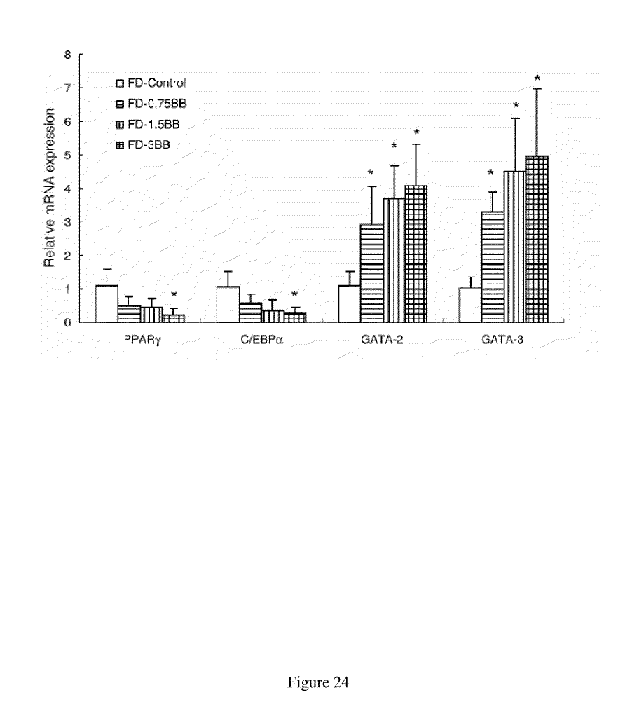

In previous studies it appeared that berberine can alter gene and protein expression of key transcription factors involved in the inhibition of adipogenesis in the mouse cell line 3T3-L1. In order to elucidate if similar transcriptional mechanisms were involved following berberine treatment of FD fed mice, the mRNA expression of key transcriptional factors for adipocyte differentiation was examined (FIG. 24). It appeared that firstly, PPARγ mRNA expression was down-regulated following berberine treatment when compared to the control treated group (0.75, 1.5, 3 mg/kg/day with berberine reducing the PPARγ mRNA expression by 56.1%, 57.7%, and 78.3% respectively). Secondly, C/EBP-α mRNA expression also appeared to be down-regulated following berberine treatment when compared to the control group (0.75, 1.5, 3 mg/kg/day, berberine decreased the C/EBP-α mRNA expression by 46.4%, 66.1%, and 72.7% respectively). Thirdly, as seen in our murine cell line experiments, both GATA-2 and GATA-3 mRNA expression appeared to be up-regulated following berberine treatment. Concentrations of 0.75, 1.5, 3 mg/kg/day berberine appeared to increase the GATA-2 mRNA expression to 2.7, 3.4, and 3.7 fold respectively and GATA-3 mRNA expression appeared to be up-regulated following treatment with berberine at the same concentrations by 3.2, 4.3, and 4.8 fold respectively.

### E. Berberine Appeared to Increase GATA-3 Protein Expression and Decreased the PPARγ Protein Expression in Epididymal Fat of FD Mice.

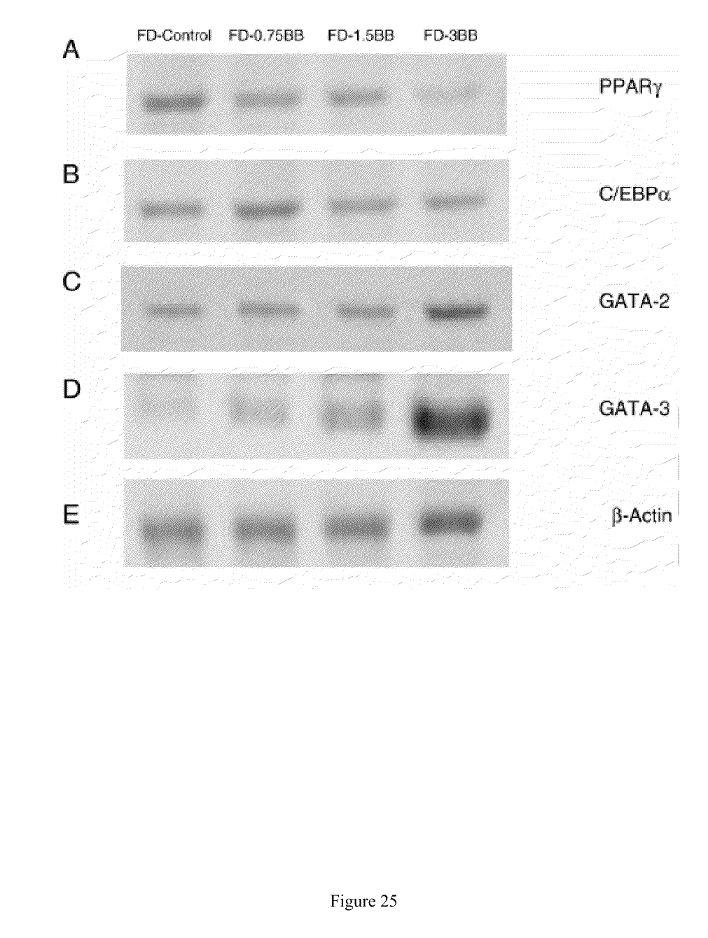

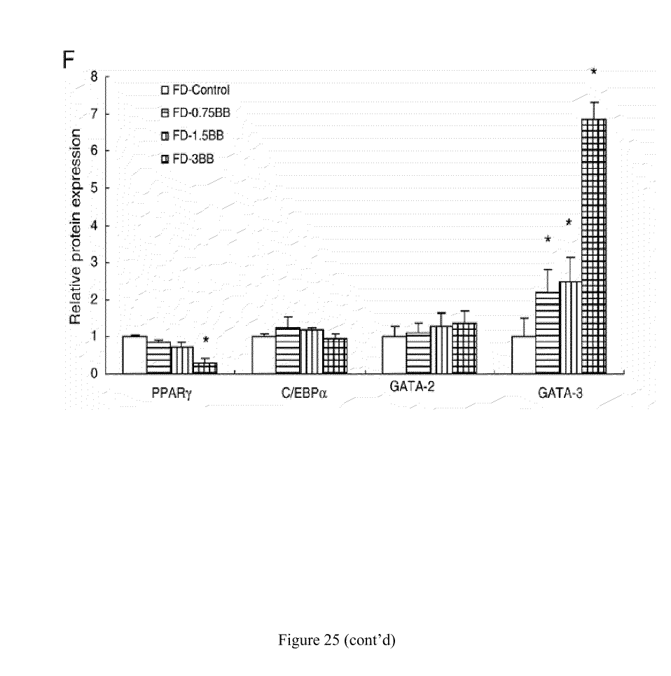

To further understand possible molecular mechanisms for the inhibition of adipogenesis by berberine, protein expression levels for several adipocyte markers were analyzed by immunoblot analysis. Decreased protein expression of PPARγ was observed in berberine treated FD mice (FIG. 25A, E). Quantification of PPARγ protein expression appeared to be relative to β-actin and normalized by the control treated group indicated that treatment with 0.75, 1.5, 3 mg/kg/day berberine appeared to decrease the PPARγ protein expression by 15%, 26.7%, and 69.3% respectively (FIG. 25F). There also appeared to be an increased protein expression of GATA-3 in berberine treated FD mice (FIG. 25D, E). Quantification of GATA 3 protein expression appeared to be relative to β-actin and normalized to the control treated group indicated that treatment with 0.75, 1.5, and 3 mg/kg/day berberine appeared to increase the GATA-3 protein expression to 2.2, 2.5, and 6.9 fold respectively (FIG. 25F). However, the quantitative protein expression of CEBP-α and GATA-2 did not appear to be significantly changed by berberine treatment (FIG. 25B, C, F).

### Discussion:

There is overwhelming evidence that obese individuals have a substantially higher risk of developing many diseases such as type 2 diabetes, hyperlipidemia, cardiovascular disease and hypertension. Thus the quest for possible compounds to aid in the treatment of obesity has intensified.

Berberine an herbal compound traditionally used in Chinese medicine as an anti-microbial, appears to have potential use as an anti-obesity agent. In this example, a high-fat diet-induced obesity mice model was utilized to verify the apparent inhibitory effects of berberine on adipogenesis and provide strong experimental data that berberine appeared to have an effect on other important factors known to be involved in the development of obesity.

Following berberine treatment in this mouse model of obesity, there appeared to be a reduced increase in mouse weight and food intake along with a reduction in the ratio of epididymal fat to total weight, as well as the ratio of liver weight to total weight. In addition, blood glucose, triglyceride and total cholesterol levels in high-fat diet induced obesity mice appeared to be all lowered, indicating berberine may be a potential natural compound for the treatment of obesity. Interestingly, berberine did not produce any physiological changes in normal diet mice. Such a difference may be due to the pivotal role PPARγ plays in adipogenesis, since the results show that berberine appears to decrease PPARγ mRNA and protein expression in FD mice but not in ND mice.

Previous examples indicate that berberine appears to increase GATA-2 and 3 gene and protein expression during the inhibition of differentiation of murine 3T3-L1 preadipocytes and human white preadipocytes. In this example, the results indicate for the first time that berberine appears to increase both the gene and protein expression of GATA-3 even though the protein expression of GATA-2 was not significantly changed.

An interesting finding in this study is that berberine also appeared to cause a decrease in the food intake in FD mice along with decreased weight gain.

The limitation of most anti-obesity compounds is their significant toxicity. It has previously been demonstrated that berberine appears to have low toxicity to 3T3-L1 mouse preadipocyte and no toxicity in human white preadipocyte. In this mouse model the results indicated that berberine did not appear to affect the weight either of the kidney or the spleen relative to body weight in FD mice and did not appear to exhibit any obvious toxicity to FD mice organs or to ND mice. From this study, it appears that berberine has excellent potential as an effective anti-obesity agent with no obvious toxicity.

### Example 5

### Berberine and Evodimine Enhanced SERT Expression and Promoter Activity Depending on 5-HTTLPR Polymorphism in RN46A Cells

### Materials and Methods

Cell culture: Raphe-derived neuron cell line RN46A was graciously provided by Scott R. Whittemore (University of Louisville School of Medicine). Cells were cultured at 33° C. in a humidified 5% CO₂ atmosphere. 2×10⁵ cells/well (24 well plate) were cultured in 0.5 ml/well cell culture medium (DMEM with 10% FBS, 100 units/ml penicillin, 100 μg/ml streptomycin, and 250 μg/ml G418) for 2 days, then medium was changed to cell culture medium (DMEM with 10% fetal calf serum, without antibodies) for 2 days, with the start of transfection at the 5th day.

RN46A cell line was graciously provided by Scott R. Whittemore (University of Louisville School of Medicine). Dulbecco's modified Eagles medium (DMEM): Nutrient Mixture F-12 Ham (Sigma-Aldrich). Fetal Bovine Serum (FBS) was purchased from Atlanta Biologicals Corp. Penicillin/streptomycin, G418 were ordered from Sigma-Aldrich. luciferase reporter vector pGL4.10 (firefly luciferase) (Promega) pRL-SV40 vector (renilla luciferase) (Promega) Lipofectamine 2000 (Invitrogen) Opti-MEM®69I Reduced Serum Medium (Invitrogen) Dual-Luciferase® Reporter Assay System (Promega) 20/20ⁿ Luminometer (Turner Biosystems, Sunnyvale Calif. 94085).

MTT assay: To detect the effect of berberine, evodiamine and their combination on the viabilities of RN46A cells, RN46A cells were plated in 96-well culture plates at a density of 4×10³ cells/well and cultured in medium using methods mentioned above, supplemented with varying concentrations of berberine and evodiamine, alone and in combination. Medium was removed at different time points and MTT (0.5 mg/ml in NM, 50 μl/well) was added. The plates were incubated at 37° C. for 4 hours, followed by the addition of DMSO (150 μl/well), and incubated at 37° C. for 1 hour. Optical density (OD) was measured at 570 nm with 650 nm as background.

Realtime RT-PCR: Real-Time reverse transcriptase polymerase chain reaction (RT-PCR) analysis was used to measure mRNA expression of SERT under the control of β-actin. Briefly, total RNA was isolated from RN46A cells following treatment with Trizol reagent (Invitrogen, Carlsbad, Calif., USA) according to the manufacturer's instruction. RNA was quantified using absorption of light at 260 and 280 nm, and sample integrity was checked by 1.5% agarose gel electrophoresis. 0.8 μg of total RNA from each sample was used for reverse transcription reaction using the TaqMan Reverse Transcription Reagents (Applied Biosystems, Foster City, Calif., USA) following the manufacturer's instructions. PCR was done using the SYBR green Master Mix (Applied Biosystems, Foster City, Calif., USA) following the manufacturer's instructions. The primers (Integrated DNA Technologies, Coralville, Iowa, USA) sequences are shown in Table 1. The temperature cycling conditions of amplification were as follows: an initial step of denaturation at 95° C. for 10 min, 40 cycles of denaturation at 95° C. for 30 s, annealing at 55° C. for 30 s, and elongation at 72° C. for 30 s. Real-Time RT-PCR was performed using Mx3000P Real-Time Thermocyclers (Statagene, La Jolla, Calif., USA). The relative mRNA levels of these genes were calculated by Pfaff s mathematical method (Pfaffl, MW, 2001) and normalized with control-treated groups.

Immunoblot Analysis: Protein expression of SERT was assessed by Western Blot. Total proteins were isolated from RN46A cells treated with compounds or vehicles. The cells were washed twice with PBS, lysed in a lysis buffer containing HEPES-Tx-PI buffer: 910 mM HEPES, 5 mM EDTA, 350 mM Sucrose, 1 μg/ml leupeptin, 1 μg/ml pepstatin, 1% Triton-X, 1 mM PMSF), processed with a 25G needle and centrifuged at 10,000 rpm for 5 min. Protein concentration was determined using the Micro BCA Protein Assay Kit (Fisher Scientific, Pittsburgh, Pa., USA) according to the manufacturers instructions. 25 μg proteins were loaded and separated by electrophoresis using the BioRad Electrophoretic System (60 V for 1 hour followed by 90 V for 5 hours). The proteins were then transferred to nitrocellulose membranes at 35 V overnight. The membrane was blocked for 1 hour at room temperature in 5% nonfat milk in Tris-buffered saline (TBS), then washed with Tween-20-TBS (TTBS, 0.1% Tween-20) three times (15 min, 5 min, 5 min), followed by incubation with primary antibodies SERT (dilution 1:200) (Antibody was purchased from Santa Cruz Biotechnology, Santa Cruz, Calif., USA) and β-actin (dilution 1:1000) (Antibody was purchased from Sigma-aldrich, USA) at room temperature for 2 hours. The membrane was rinsed three times (15 min, 5 min, 5 min), then incubated at room temperature for 1 hour with Horseradish peroxidase-conjugated second antibody (1:5,000). The membrane was then incubated in ECL reagent (GE Healthcare, Piscataway, N.J., USA) for 1 minute. Protein was visualized using the UVP image analysis system (UVP, Upland, Calif., USA).

### Plasmid Construction:

The PCR reaction to amplify the 5-HTTLPR alleles for sequencing and creation of reporter constructs contained primers different than those used for the genotyping experiment.

These primers amplify a larger region of 5-HTTLPR making it more useful to determine promoter strength. The primer sequences were described previously and are as follows: Forward primer 5′-GGCGTTGCCGCTCTGAATGC-3′ (SEQ ID NO:25) and Reverse primer 5′-GAGGGACTGAGCTGGACAACCAC-3′ (SEQ ID NO:26) (Hu et al., Am J Hum Genet (2006) 78(5):815-826). The PCR products of the six 5-HTTLPR alleles (S, LG, LA, XS, XL-17, and XL-18) were subcloned into the TOPO TA cloning vector pCR2.1-TOPO (Invitrogen) and transformed to One Shot TOP10 competent E. coli cells according to the manufacturer's instructions. In the case of individuals heterozygous for 5-HTTLPR, resulting transformants were screened for the presence of the correct insert using the restriction enzyme EcoR I.

### DRL Assay:

The resulting constructs, created and sequenced, in the pCR2.1-TOPO vector (S, L_{G}, L_{A}, XS, XL-17, and XL-18 alleles) were subcloned to the luciferase reporter vector pGL4.10 [luc2] (Promega) using standard molecular techniques. Additionally, each allele was cloned into pGL4.10 [luc2] in the backwards orientation to ensure that transcriptional effects were the result of consensus promoter sequences. The RN46A raphe-derived cell line was graciously provided by Scott R. Whittemore (University of Louisville School of Medicine). The co-transfection with the 5-HTTLPR pGL4.10 constructs (firefly luciferase) and the pRL-SV40 vector (renilla luciferase) to the RN46A raphe-derived cell line using Lipofectamine 2000 (Invitrogen) was described previously [10]. Expression was measured using the Dual-Luciferase Reporter Assay System (Promega) 24 h after transfection with passive lysis buffer.

Relative light units (RLU) for both the firefly and renilla luciferase was measured using a 20/20ⁿ Luminometer (Turner Biosystems, Sunnyvale Calif. 94085) and the ratio between the firefly RLU to renilla RLU in each sample was determined. The values were all normalized to the (S) allele.

Statistical analysis: The data was analyzed by the ANOVA procedure of Statistical Analysis System (SAS Institute, 1999-2001). Significant differences between groups were determined using Student's t-test or Duncan's multiple range tests at the p<0.05 (*).

### Results

### MTT Assay

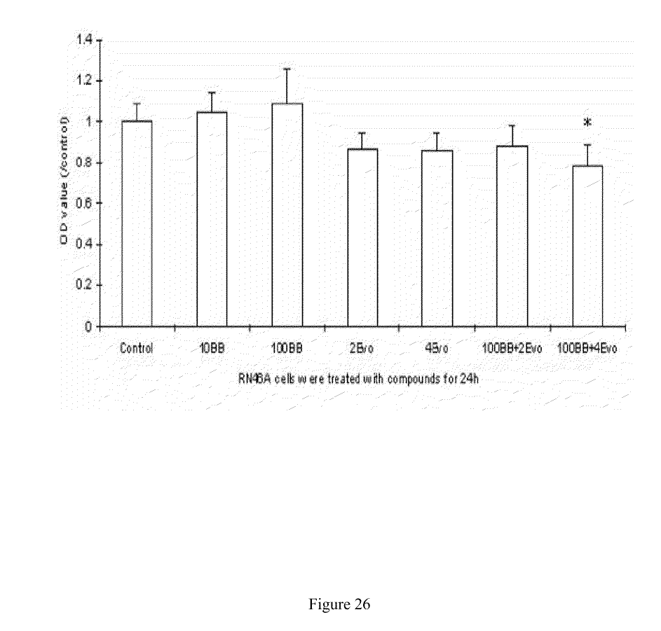

The results demonstrated that berberine (10 and 100 μM), low concentration of evodiamine (2 and 4 μM), and one combination (100 μM berberine and 2 μM evodiamine did not appear to decrease cell viabilities significantly. However, 100 μM berberine combined with 4 μM evodiamine appeared to inhibit cell viabilities significantly (FIG. 26).

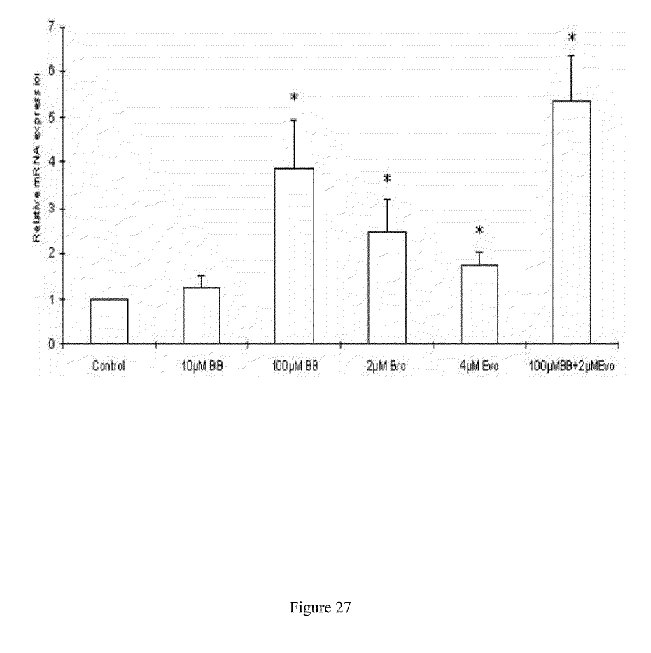

Compared to control group, 10 μM berberine, 100 μM berberine, 2 μM evodiamine, 4 μM evodiamine, and 100 μM berberine+2 μM evodiamine appeared to increase SERT mRNA expression by 1.3, 3.9, 2.5, 1.7, and 5.4 fold respectively (FIG. 27).

### Immunoblot Analysis

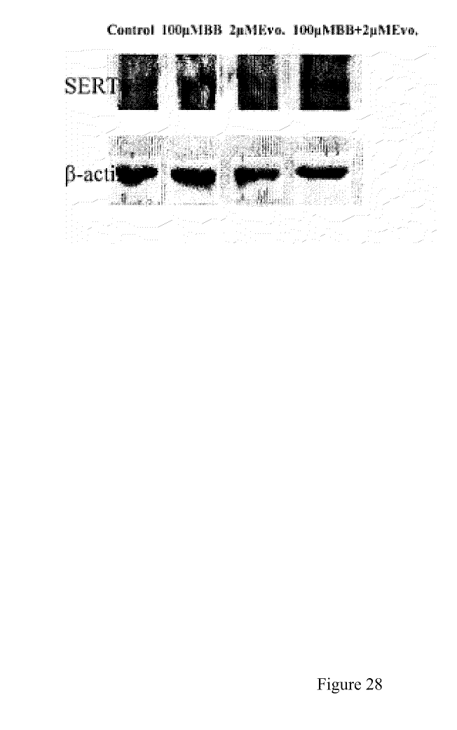

Western blot results are very consistent with real-time RT-PCR results, which mean the SERT protein expression profile appears to be affected by berberine and evodiamine and that is consistent with its mRNA expression profile affected by berberine and evodiamine. (FIG. 28).

### Plasmid Construction

The 5-HTTLPR alleles were subsequently sequenced in both directions using the M13 forward and M13 reverse primers specific to the pCR2.1-TOPO vector using BigDye Terminator v3.1 Cycle Sequencing Kit according to the manufacturer's instructions (Applied Biosystems).

### DRL Assay

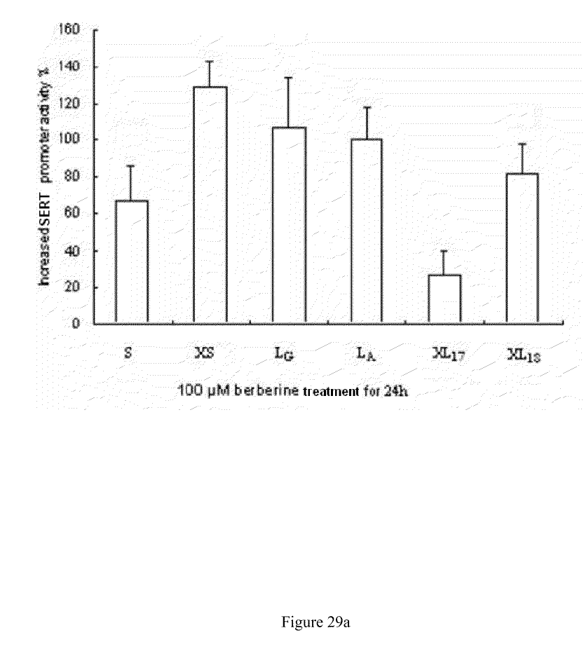

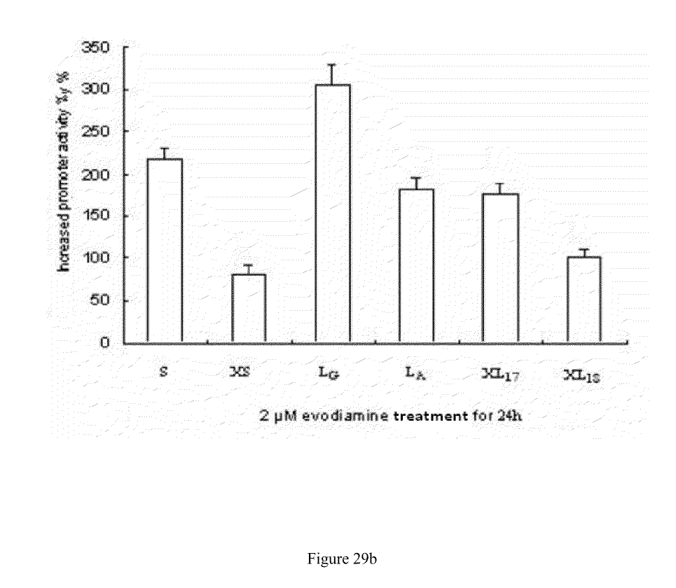

Comparing to controls (vehicle-treated groups), 100 μM berberine appeared to increase SERT promoter activities by 67%, 128.7%, 106.9%, 100.4%, 26.2%, and 82% in S allele, XS allele, L_{G} allele, L_{A} allele, XL17 allele, and XL18 allele respectively. Same concentration of berberine enhanced SERT promoter activity differently depending on the 5-HTTLPR polymorphism in RN46A cells (FIG. 29a).

Comparing to controls (vehicle-treated groups), 2 μM evodiamine appeared to increase SERT promoter activities by 206.9%, 74.3%, 288.5%, 171.5%, 165.6%, and 96.3% in S allele, XS allele, L_{G} allele, L_{A} allele, XL17 allele, and XL18 allele respectively. Same concentration of evodiamine enhanced SERT promoter activity differently depending on the 5-HTTLPR polymorphism in RN46A cells (FIG. 29b).

### Discussion:

Berberine and low concentration of evodiamine appeared to increase SERT mRNA and protein expression, enhanced SERT promoter activity differently depending on the 5-HTTLPR polymorphism, at the same time, appeared to exhibit no obvious cytotoxicity in RN46A cells.

### Example 6

### Amentoflavone Enhanced Inhibitory Effects of Berberine on Adipogenesis During Preadipocytes Differentiation

### Materials and Methods

### Cell Culture and Drug Treatment:

3T3-L1 cells (American Type Culture Collection, Manassas, Va., USA) were cultured at 37° C. in a humidified 5% CO₂ atmosphere and grown in a DMEM (Sigma-Aldrich, St. Louis, Mo. USA) supplemented with 10% Fetal Bovine Serum (FBS, Atlanta Biologicals Corp., Lawrenceville, Ga., USA), 100 units/ml penicillin and 100 μg/ml streptomycin. Differentiation was induced by addition of 167 nM insulin, 520 μM isobutylmethylxanthine (IBMX), and 1 μM dexamethasone for 2 days and then only with 167 nM insulin for 6 days. 25 μM amentoflavone combined with various concentrations of berberine was added to the differentiation medium with or without 15 μM clozapine at the beginning of differentiation induction for 8 days to observe their effects.

Oil-Red-O staining and quantification: Oil-Red-O Staining and its quantification were performed as previously reported. Briefly, medium was removed and cells were washed with PBS twice, fixed with 3.7% formalin at room temperature for 30 min, then rinsed with pure water, added 60% isopropanol and incubated for 5 minutes, then moved out isopropanol and stained cells with diluted Oil-Red-O solution (Oil-Red-O store solution (3 mg/ml in pure isopropanol): water=3:2) at room temperature for 10 min. Then the diluted Oil-Red-O solution was removed and cells were washed 3 times with pure water. After staining, the cells were washed twice with 70% ethanol to remove excess stain. Stained oil droplets in the cells were dissolved in isopropanol containing 4% Nonidet-P40 (Fisher Scientific, Pittsburgh, Pa., USA) shaking in 37° C. at speed 100 rpm for 1 hour, and OD values were measured at an absorbance of 490 nm and then normalized with control group.

### Results:

### Oil-Red-O Staining and Quantification:

In order to study the potential additive or synergetic effects of amentoflavone on inhibitory effects of berberine on adipogenesis during differentiation, 25 μM amentoflavone combined with various concentrations of berberine was added to the differentiation medium with or without 15 μM clozapine for 8 days during the culture of 3T3-L1. Differentiation was monitored by Oil-Red-O staining as previously described.

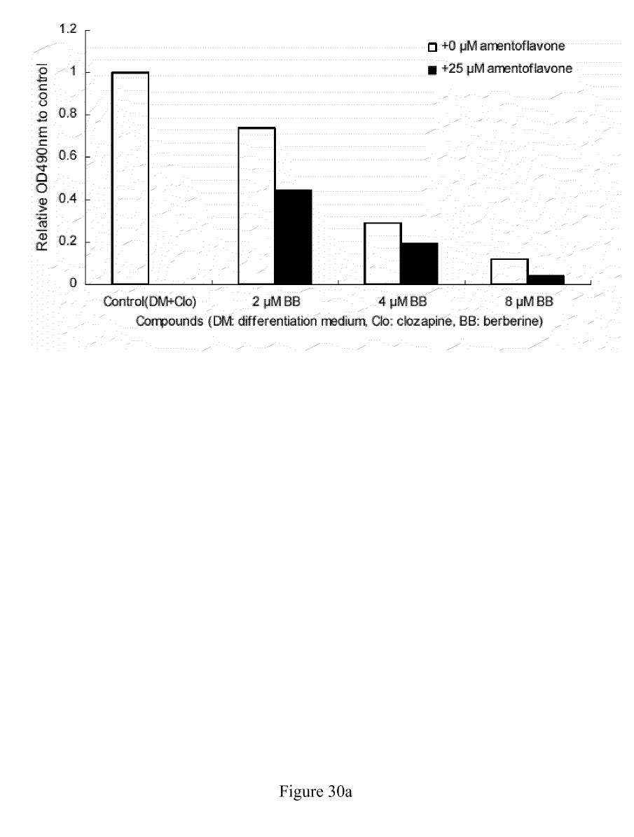

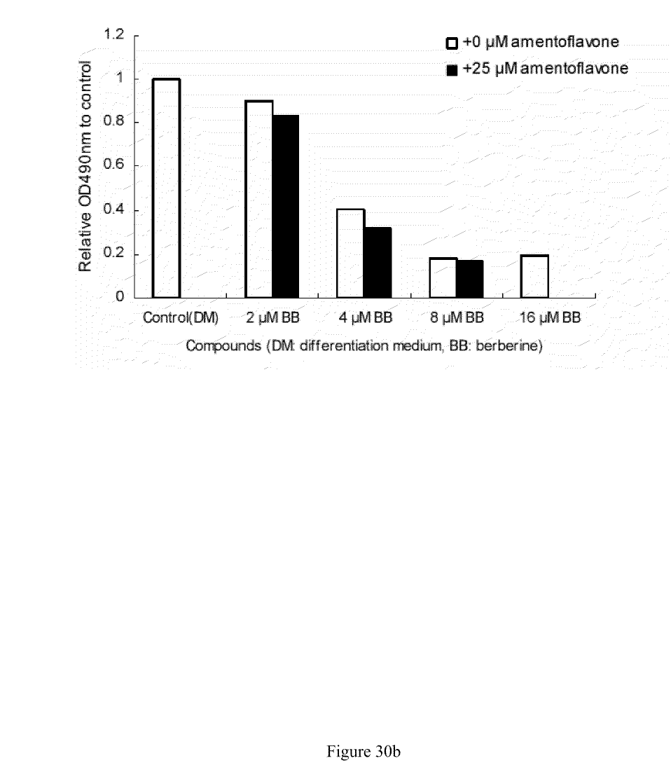

Quantitative Oil-Red-O staining measurements (FIG. 30a) indicated when compared with no berberine treated groups (3T3-L1 cells grown in differentiation medium adding 15 clozapine), 2, 4, 8 μM berberine treatment for 8 days indicated a reduction in lipid content by 26%, 70.7%, 88.1% respectively. However, more interestingly, the combination of 2, 4, 8 berberine with 25 μM amentoflavone treatment inhibited adipogenesis by 55.7%, 80.8%, and 95.9% respectively which illustrated that the addition of 25 μM amentoflavone enhanced the inhibitory effects of berberine by 29.7%, 10.1% and 7.8% respectively.

Quantitative Oil-Red-O staining measurements (FIG. 30b) indicated when compared with no berberine treated groups (3T3-L1 cells grown in differentiation medium), 2, 4, 8, 16 μM berberine treatment for 8 days indicated a reduction in lipid content by 9.9%, 59.6%, 81.7%, and 81% respectively. Interestingly, the combination of 2, 4, 8 μM berberine with 25 μM amentoflavone treatment inhibited adipogenesis by 16.7%, 68.4%, and 83% respectively which illustrated that the addition of 25 μM amentoflavone enhanced the inhibitory effects of berberine slightly.

Discussion:

Taken together, the addition of 25 μM of amentoflavone appeared to enhance the inhibitory effects of berberine on adipogenesis during 3T3-L1 differentiation induced by differentiation medium with or without clozapine, which suggests the combination of berberine with amentoflavone may exhibit obesity prevention effects.

### Example 7

### Human Clinical Trial 1

Hypothesis: Subjects taking 500 mg of Berberine with every meal (TID) will stimulate GATA-2 and GATA-3 transcription factors, which will in turn suppress development of adipocytes, causing subjects to lose weight and change body composition.

Objectives: The primary objective of this pilot study will be to evaluate the safety and effectiveness of Berberine on weight reduction and change in body composition in an obese population.

The secondary parameters are to evaluate changes in the following labs: glucose, cholesterol levels (triglycerides, LDL, HDL and total cholesterol), Cortisol, ACTH, appropriate sex hormone and inflammatory markers (CRP, Sed rates).

Study Population: Subjects who have given informed consent to participate in the clinical study and have met all the criteria required for entry into the clinical study may participate in the study. There will be a total of 60 subjects enrolled, 30 per treatment group. Randomization to groups will be by gender to allow for an equal number of males and females in the control and treatment groups.

Patient Eligibility The following criteria must be met:

### Inclusion Criteria:

1. Males and females 18 years and older.
2. BMI of 30 or greater.
3. Agree to keep diet, exercise and all current health habits stable during participation in the study.
4. Willing to sign consent and follow study-related procedures.

### Exclusion Criteria:

1. Gastroparesis or intestinal pseudo-obstruction.
2. Receiving medications known to affect gastric motility.
3. If currently on cholesterol-lowering, diabetes or thyroid medication, must agree to be on a stable dose throughout the duration of the study.
4. Currently taking any antipsychotic medications (Abilify/Aripiprozole; Respiradone/Risperdal; Olanzapine/Zyprexa; Seroquel/Quentiapine; Geodon/Ziprazadone; Clozapine/Clozaril).
5. Taking prescription or over-the-counter appetite suppressants, herbal products or other medications for weight loss within 1 month of enrollment and agree not to start such products while participating in the study.
6. Using acupuncture for weight loss.
7. Severe eating disorders such as bulimia or binge eating.
8. Obese due to a clinically diagnosed endocrine problem.
9. Pregnant (proven by positive (3hCG) or lactating.
10. History of anemia (<10 g/dl) over past 3-months.
11. Patients with significantly abnormal hepatic liver function tests or renal disease.
12. Prior bariatric surgery.
13. History of peptic ulcer disease.
14. Use of another investigational device or agent in the 30 days prior to enrollment.
15. Participation in another clinical study.
16. Life threatening co-morbidity or life expectancy of less than one year.
17. Myocardial Infarction or one or more episodes of unstable angina within six months prior to enrollment.
18. Implanted with a permanent pacemaker, an automatic implantable defibrillator, or other electro-stimulation device.
19. Use of prescription, over-the-counter or herbal weight loss products.
20. In the opinion of the investigator, this subject will not comply with the protocol or is in some other way not appropriate for this study.

Prohibited Medications:

Any medication used for weight loss during the study will be prohibited. This includes herbal formulas, prescription and over-the-counter medications. Some examples include, but are not limited to, the following: Meridia, Orlistat, Chitosan, or Ephedra.

Any antipsychotic medications, including the following medications, will not be allowed during the study: Abilify/Aripiprozole; Respiradone/Risperdal; Olanzapine/Zyprexa; Seroquel/Quentiapine; Geo don/Ziprazadone; Clozapine/Clozaril).

Cholesterol, thyroid, diabetes and steroids medications taken during the study will be restricted to stable doses. Any subject who needs modification of medication due to any reason will be evaluated on a case-by-case basis, and may be asked to withdraw from the study.

Study Design: This is a randomized, double-blind, placebo-controlled, parallel-group study of 500 mg IHBG-10 taken 15 minutes before the 3 main meals of the day, to evaluate whether subjects will lose weight and change body composition in 12 weeks.

Generally healthy, obese, subjects will be randomized at a 1:1 ratio to placebo or 500 mg IHBG-10.

Subjects will ingest study drug up to three times daily with meals to determine whether those subjects receiving IHBG-10 will have a decrease in weight, body fat, glucose levels, and a change in waist-to-hip ratios.

Subjects will be enrolled in the study approximately 14 weeks and will be on treatment or placebo for 12 weeks. Subjects will have up to 4 visits. Visit 1 will be a screening visit to determine eligibility. Subjects will return in one week for Visit 2 (Randomization). Visits 3 and 4 will occur at 6-week intervals. Subjects will be asked to adhere to the visit schedule as closely as possible. Subjects who are not able to comply with the study visits will be asked to withdraw from the study based on the investigator's discretion.

All subjects will sign an informed consent prior to enrollment into the program. Subjects will be asked to keep diet, exercise and all current health habits stable for the next 12 weeks while in the study.

The study visits will include the following procedures:

### Screening Visit (Visit 1):

Subjects will visit the clinic and complete the following:
1. Informed consent.
2. Subjects will be given a diet diary to be maintained over the next week and will be returned to the clinic at their next scheduled visit.
3. Medical and dietary histories will be obtained.
4. Current prescription, over-the-counter, and nutraceutical medications will be collected for medications taken within the last 30 days.
5. Brief physical exam (heart/lungs/abdomen) will be performed.
6. Labs will be completed for the following:
   a. Complete Metabolic Panel (CMP)
   b. Complete Blood Count (CBC)
   c. Lipid Panel (total cholesterol, LDL, HDL, triglycerides)
   d. Thyroid (TSH, Free T4)
   e. Serum HCG for all females regardless of age.
7. Vital signs (blood pressure, pulse) will be measured.
8. Weight and height will be measured.
9. Body Mass Index (BMI) will be assessed.
   Subjects will have a whole body (DXA) scan scheduled to measure body fat prior to their next visit.

Visit 2 (2 Week after Visit 1, +/−7 Working Days):
1. Informed Consent will be reviewed.
2. Lab work from the previous visit will be discussed with the patient.
3. The diet diary will be reviewed and a new one will be dispensed (7 consecutive days to be filled out prior to the next visit).
4. Adverse Events or Serious Adverse Events will be collected and assessed. (A physical exam will be conducted if symptoms require further evaluation.)
5. Changes in Concomitant medications will be reviewed.
6. Vital Signs will be measured (blood pressure, heart rate, weight).
7. EKG will be completed.
8. Labs will be collected for the following:
   a. Cortisol, ACTH
   b. Sex hormone (estrogen, progesterone, testosterone)
   c. Vitamin D-1,25 dihydroxy
   d. Immune assay (SED, CRP)
   e. mRNA for analysis of GATA 2, GATA-3, PPARγ, C/EBPα and SREBP-1
   f. DNA will be collected for HTR 2C and HTR2A
   g. Serum pregnancy test if indicated.
9. Subjects will fill out Activity Level Questionnaire.
10. Waist and hip measurements will be documented.
11. Waist-to-hip ratio will be calculated.
12. Body fat will be measured by whole body DXA scan.
13. Body mass index will be assessed.
14. Study medication will be dispensed and patients will be instructed to take one pill 15 minutes prior to each meal to total 3 pills per day.

Visit 3 (6 weeks after visit 2, +/−7 days):
1. Subject's Informed Consent will again be reviewed.
2. The diet diary will be collected and a new one dispensed.
3. Adverse Events or Serious Adverse Events will be collected and assessed. (A physical exam will be conducted if symptoms require further evaluation.)
4. Drug accountability will be completed. Subjects need to be >80% compliant with medications, or need to be reinstructed on medication use.
5. Study medication will be dispensed.
6. Concomitant medications will be reviewed.
7. The following measurements will be completed:
   a. Vital Signs (blood pressure, heart rate)
   b. Weight
   c. Waist and Hip measurements (hip-to-waist ratio)
   d. EKG
   e. BMI
8. The following labs will be completed:
   a. mRNA for analysis of GATA 2, GATA-3, PPARγ, C/EBPα and SREBP-1
   b. CMP
   c. CBC
   d. Cortisol
   e. ACTH
   f. Immune assay (SED rate, CRP)
   g. Serum pregnancy test if indicated.

Visit 4 (12 Weeks after Visit 2, +/−7 Days):
1. Subject's Informed Consent will again be reviewed.
2. The diet diary will be collected and reviewed.
3. Drug accountability will be completed. Subjects need to be >80% compliant with medications, or need to be reinstructed on medication use.
4. Concomitant medications will be reviewed.
5. Subjects will fill out Activity Level Questionnaire.
6. A physical exam will be performed.
7. The following labs will be collected:
   a. CMP
   b. CBC
   c. mRNA for analysis of GATA 2, GATA-3, PPARγ, C/EBPα and SREBP-1
   d. Lipid Panel (total cholesterol, triglycerides, LDL, HDL)
   e. Thyroid (TSH, Free T4)
   f. Immune assay (SED rate, CRP)
   g. Cortisol
   h. ACTH
   i. Serum pregnancy test if indicated.
8. The following measurements will be obtained and recorded:
   a. Vital Signs (blood pressure, heart rate, weight)
   b. EKG
   c. Body Fat measurement by whole body (DXA) scan
   d. Waist and Hip measurements
   e. Waist-to-hip ratio
   f. BMI

| Visit number | 1 | 2 | 3 | 4 |
|---|---|---|---|---|
| Day | 0 | 7 | 49 | 91 |
| week | 0 | 1 | 6 | 12 |
| informed consent | x | | | |
| Medical History | x | | | |
| Dietary History | x | | | |
| Physical Exam | x | | | x |
| informed consent reinforcement | | x | x | x |
| Activity Questionnaire | | x | x | x |
| Dispense Diet Diary | x | x | x | |
| Collect Diet Diary | | X | X | x |
| Dispense medication | | x | x | |
| Perform Drug accountability | | | X | X |
| Concomitant Medications | x | x | x | x |
| Whole body deta for body fat percentage | | x | | x |
| Assess Adverse Events | | | X | X |
| Waist and Hip measurements | | x | x | x |
| Waist to hip ratio | | X | x | x |
| Weight | x | x | x | x |
| Height | x | | | |
| BMI | x | X | x | x |
| Vital Signs (blood pressure, pulse) | x | x | x | x |
| CMP | x | | x | x |
| CBC | X | | X | x |
| genetic analysis- GATA2 and 3, PPARγ, | | x | x | x |
| SREBP-1, C/EBPα | | | | |
| Lipids (total cholesterol, trigylcerides, LDL, | x | | | x |
| HDL) | | | | |
| Thyroid (TSH, FT4) | x | | | x |
| Serum Pregnancy test on all females | X | X* | X* | X* |
| Sex Hormone (estrogen progesterone and | | X | | x |
| testosterone) | | | | |
| SED rate | | x | x | x |
| CRP | | x | x | x |
| Cortisol (am), ACTH | | X | x | x |
| EKG | | x | x | x |
| Vitamin D | | X | | x |
| DNA for HTR 2C and HTR 2A | | x | | |

x* Female subjects who suspect they may be pregnant during the study will have a serum pregnancy test completed.

Questionnaire: The activity questionnaire will contain the following questions and will be used to assess if subjects have maintained their habits during the study.

### Exercise Questionnaire

| Question | Response | | |
|---|---|---|---|
| Are you currently exercising? | Yes | No | |
| If you are not currently exercising, how long has | | | |
| it been since you have worked out regularly | | | |
| (weeks, months, years)? | | | |
| If you are currently exercising, how long have | | | |
| you been doing your current fitness routine | | | |
| (weeks, months, years)? | | | |
| How many days/week do you currently | | | |
| exercise? | | | |
| How long is each session of activity? | | | |
| What level of intensity is your typical | Light | Moderate | Heavy |
| activity session? | | | |
| What type of activity do you generally do for | | | |
| your activity session? | | | |
| Where do you exercise (home, gym, etc)? | | | |

| Level of | | |
|---|---|---|
| Intensity | RPE | Physical Cues |
| Light | Easy | Does not induce sweating unless it's |
| | | a hot, humid day. There is no |
| | | noticeable change in breathing |
| | | patterns. |
| Moderate | Somewhat | Will break a sweat after performing |
| | hard | the activity for about 10 minutes. |
| | | Breathing becomes deeper and more |
| | | frequent. You can carry on a |
| | | conversation but not sing. |
| Heavy | Hard | Will break a sweat after 3-5 minutes. |
| | | Breathing is deep and rapid. You can |
| | | only talk in short phrases. |

Diet Log:

Subjects will keep a diet log of everything they eat and drink for seven consecutive days between visits 1-2, 2-3, and visits 3-4. Evaluation of the total number of calories, fat, carbohydrates, and protein will be assessed.

### Laboratory Analysis:

At the screening visit, a total of 10 ml (2 teaspoons) of blood will be drawn for the following tests: CMP, CBC, Lipid panel, Thyroid (TSH, Free T4), and beta HCG.

The randomization visit (Visit 2) will include at total of 24 ml (2 tablespoons) of blood to be drawn and will include the following tests: Cortisol, ACTH, estrogen, progesterone, testosterone, Vitamin D, SED, CRP, mRNA for genetic analysis of GATA2 and GATA 3, PPARγ, SREBP-1, C/EBPα, DNA for HTR 2C and HTR 2A, serum pregnancy test if indicated.

Visit 3 labs will include CMP, CBC, mRNA, Cortisol, ACTH, SED rate, CRP, and serum pregnancy test if indicated. The total amount drawn at this visit will include 20 ml (1½tablespoons).

Visit 4 will include CMP, CBC, mRNA, Cortisol, ACTH, Lipid Panel, TSH, Free T4, Vitamin D, and Immune assay. The total amount of blood drawn will include 12 ml (2 tablespoons (tsp)). A Comprehensive Metabolic Panel (CMP) will include BUN, Albumin, ALT, AST, Total Bilirubin, Calcium, Carbon Dioxide, Chloride, Creatinine, Glucose, Alkaline Phosphatase, Potassium, Total Protein, and Sodium. A Lipid Panel will include total cholesterol, triglycerides, LDL, HDL, and LDL/HDL ratio. An Immune assay will include IL-6, TNFα, Sed rate, C-reactive protein. A Complete Blood Count (CBC) will include RBC; WBC; Hemoglobin; Hematocrit; Platelet Count; RBC Indices: MCV, MCH, MCHC; and Automated 5-part WBC Differential.

Subjects will need to be fasting for the CMP (on visits 1, 3, and 4).

Serum pregnancy tests will be performed on all females prior to randomization. For the duration of the study, in case of suspicion of pregnancy, another serum pregnancy test will be performed.

### Study Medication and Placebo:

Avera McKennan Hospital Pharmacy will compound IHBG-10 and placebo in 250 mg capsules. Subjects will take two capsules 15 minutes prior to any meals. The placebo will be a visual match to active ingredient capsules. An unblinded study pharmacist will randomize the subjects and dispense the study medication or placebo. Subjects will receive study medication or placebo at Visit 2 (randomization) and Visit 3. At Visit 3 and Visit 5 (final visit), subjects will return the previously dispensed capsules. Study medication previously dispensed to a subject and returned medication may not be re-dispensed to another subject. Medication compliance will be assessed at Visit 3 and Visit 4.

### Blinding:

Only in the case of an emergency, when knowledge of the investigational product is essential for the clinical management or welfare of the subject, will the investigator unblind a subject's treatment assignment. The investigators of this study will record the date and reason for revealing the blinded treatment assignment for that subject.

Subjects will be sent letters at the conclusion of the study regarding their final laboratory work and unblinding information. At the principal investigator's discretion, any subject with laboratory measures outside of the range of normal that is in need of follow-up will be contacted by phone or subject's stated preferred method of contact.

### Concomitant Therapies and Non-Drug Therapies:

All concomitant medications taken since informed consent and during the study will be recorded in the source document. Subjects will be encouraged to maintain a steady dose of concomitant medications throughout the study. All changes in concomitant medications or adjustments to dosage will be recorded in the source document. Subjects will be encouraged to maintain consistency with their diet, exercise habits, alcohol consumption, and smoking habits throughout the study.

### Subject Completion and Withdrawal:

Subjects will be considered to have completed the study when they have attended all visits, and all assessments have been performed.

Any subject who enters the study but does not complete the study according to the above definition will be considered a withdrawal, irrespective of whether they have received any study medication.

Some reasons why a subject may withdraw from the study may include, but are not limited to the following situations:
A subject may request withdrawal from the study.
If in the investigator's opinion, continuation in the study would be detrimental to the subject's well being.
The sponsor requests the subject's withdrawal.

When a subject withdraws from the study, all medication and study devices should be returned and all attempts must be made to complete a final visit. Study-related medication that is returned from subjects who have withdrawn from the study may not be re-dispensed.

### Adverse Events and Serious Adverse Events:

### Adverse Event:

An adverse event is defined as follows: Any untoward medical occurrence in the subject temporarily associated with the use of a medicinal product, whether or not considered related to the medicinal product. An AE can therefore be any unfavorable and unintended sign (including an abnormal laboratory finding), symptom or disease (new or exacerbated) temporally associated with the use of a medicinal product. For marketed medicinal products this also includes abuse or misuse of the product. All adverse events will be followed throughout the study.

### Serious Adverse Event (SAE)

A serious adverse event is any untoward medical occurrence that, at any dose that:
results in death;
is life-threatening;
requires hospitalization;
results in disability/incapacity; or
is a congenital anomaly/birth defect.

All SAEs must be reported to the Avera Institutional Review Board within ten working days of the event, or upon learning of the event.

### Study Analysis:

### Data Collection

Study staff will fill out source documentation with the specific data points as listed in the protocol. After subjects have been seen in the clinic and all data points collected, the study staff will enter the data points on a spreadsheet specifically designed for the data collection. Periodically, the data will be reviewed for accuracy as entered from the source document to the final spreadsheet. The spreadsheet will be used for statistical analysis.

### Analysis

Descriptive statistics, such as means, standard deviations and statistical distributions, will be explored for the weight, BMI, and lab values. Further comparisons will be completed as determined during the analysis of lab and other outcomes. Statistical analysis of variance and covariance will be use to compare these outcome measures between intervention and control groups and to test whether there is a significant difference. Other advanced statistical models will be employed depending on the statistical distributions observed.

### Ethical Conduct of the Study:

This study will be conducted in accordance with “good clinical practice” (GCP) and all applicable regulatory requirements including, where applicable, the October 1996 version of the Declaration of Helsinki

All applicable regulatory documents will be reviewed by the Avera Institutional Review Board and will have received approval prior to any implementation of the protocol procedures.

Informed consent will be obtained before the subject can participate in the study. The contents and process of obtaining informed consent will be in accordance with all applicable regulatory requirements.

### Example 8

### Human Clinical Trial 2

### Hypothesis:

Subjects who are taking antipsychotics (Seroquel/Quentiapine, Zyprexa/Olanzapine and/or Risperdal/Risperidone) in combination with 500 mg of (Berberine) IHBG-10 at every meal (TID) will stimulate GATA-2 and GATA-3 transcription factors, which will in turn suppress development of adipocytes, causing subjects to maintain or lose weight and/or change body composition, while taking their regular antipsychotic medications.

### Objectives:

The primary objective of this study is to evaluate the safety and effectiveness of IHBG-10 on weight reduction and change in body composition in subjects who are taking stable doses of Seroquel/Quentiapine, Zyprexa/Olanzapine or Risperdal/Risperidone.

The secondary parameters are to evaluate changes in the following labs: HbA1c, fasting glucose, glucose tolerance tests (ogtt), cholesterol levels (triglycerides, LDL, HDL and total cholesterol), CRP, and SED rates.

### Study Design/Population:

This is a real-life surveillance study to evaluate the effect of IHBG-10 on weight and body composition in subjects who are taking Seroquel/Quentiapine, Zyprexa/Olanzapine and/or Risperdal/Risperidone. These subjects will be monitored for factors associated with the parameters of the primary and secondary objectives.

Subjects who are taking stable doses of Seroquel/Quentiapine, Zyprexa/Olanzapine and/or Risperdal/Risperidone and have given informed consent for the clinical study may participate. These subjects must meet all criteria required for entry into the clinical study. There will be a total of 80 subjects enrolled: 60 will be randomized to receive IHBG-10 (20 subjects who are taking Seroquel/Quentiapine, 20 who are taking Zyprexa/Olanzapine and 20 subjects who are taking Risperdal/Risperidone). Twenty subjects who are taking Seroquel/Quentiapine, Zyprexa/Olanzapine, or Risperdal/Risperidone will receive placebo.

### Patient Eligibility:

The following criteria must be met:

Inclusion Criteria:
1. Males and females 18 years and older
2. Agree to keep diet, exercise and all current health habits stable during participation in the study
3. Willing to sign consent and follow study-related procedures
4. Subjects must be taking Seroquel/Quentiapine, Zyprexa/Olanzapine and/or Risperdal/Risperidone for 3 or more months

Exclusion Criteria:
1. Current active acute psychotic episode
2. Suicidal ideation within 12 months prior to screening
3. Gastroparesis or intestinal pseudo-obstruction
4. Receiving medications known to affect gastric motility
5. If currently on cholesterol-lowering, diabetes or thyroid medication, must agree to be on a stable dose throughout the duration of the study
6. Currently taking any antipsychotic medication other than Risperdal/Risperidone, Zyprexa/Olanzapine, or Seroquel/Quentiapine
7. Taking prescription or over-the-counter appetite suppressants, herbal products or other medications for weight loss within 1 month of enrollment and agree not to start such products while participating in the study
8. Using acupuncture for weight loss
9. Severe eating disorders such as bulimia or binge eating
10. Obese due to a clinically diagnosed endocrine problem
11. Pregnant (proven by positive (3hCG) or lactating
12. History of anemia (<10 g/dl) over past 3-months
13. Thrombocytopenia
14. Patients with significantly abnormal hepatic liver function tests or renal disease
15. Prior bariatric surgery
16. History of peptic ulcer disease
17. Use of another investigational device or agent in the 30 days prior to enrollment
18. Participation in another clinical study
19. Life threatening co-morbidity or life expectancy of less than one year
20. Subjects who have any major cardiovascular disorder, including congestive heart failure, myocardial infarction or one or more episodes of unstable angina within six months prior to enrollment
21. Implanted with a permanent pacemaker, an automatic implantable defibrillator, or other electro-stimulation device
22. In the opinion of the investigator, this subject will not comply with the protocol or is in some other way not appropriate for this study
23. Has a diagnosis of Immunodeficiency, or HIV+ status.
24. Subjects who are not able to read and write to complete necessary diaries and questionnaires.

Prohibited Medications:

Any medication used for weight loss during the study will be prohibited. This includes herbal formulas, prescription and over-the-counter medications. Some examples include, but are not limited to, the following: Meridia®, Orlistat®, Chitosan®, or Ephedra®.

Cholesterol, thyroid, diabetes and steroids medications taken during the study will be restricted to stable doses. Any subject who needs modification of medication due to any reason will be evaluated on a case-by-case basis, and may be asked to withdraw from the study.

There is some expectation of decreased need for diabetes medication as documented in several previous studies with IHBG-10. The study will monitor the changes needed.

### Procedures:

The study visits will include the following procedures:
Screening visit (Visit 1):
Subjects will visit the clinic and complete the following:
   1. Informed consent.
   2. Subjects will be given a diet diary to be maintained over the next week and will be returned to the clinic at their next scheduled visit.
   3. Medical, psychological and dietary histories will be obtained.
   4. Current prescription, over-the-counter, and nutraceutical medications will be collected for medications taken within the last 30 days.
   5. Brief physical exam (heart/lungs/abdomen) will be performed.
   Labs will be completed for the following:
      a. Complete Metabolic Panel (CMP)
      b. Complete Blood Count (CBC)
      c. Lipid Panel (total cholesterol, LDL, HDL, triglycerides)
      d. Thyroid (TSH, Free T4)
      e. Serum HCG for all females regardless of age.
      f. Vital signs (blood pressure, pulse) will be measured.
      g. Weight and height will be measured.
      h. Body Mass Index (BMI) will be assessed.
   Subjects will have a whole body (DXA) scan scheduled to measure body fat prior to their next visit.

Visit 2 (2 weeks after visit 1, +/−7 working days):
Informed Consent will be reviewed.
Lab work from the previous visit will be discussed with the patient.
The diet diary will be reviewed and a new one will be dispensed (7 consecutive days to be filled out prior to the next visit).
Adverse Events or Serious Adverse Events will be collected and assessed. (A physical exam will be conducted if symptoms require further evaluation.)
Changes in Concomitant medications will be reviewed.
Vital Signs will be measured (blood pressure, heart rate, weight).
EKG will be completed.
Labs will be collected for the following:
   a. Stress hormones Cortisol and Adrenocorticotropin hormone (ACTH)
   b. Sex hormone (estrogen, progesterone, testosterone)
   c. Vitamin D-1,25 dihydroxy
   d. Inflammatory markers (SED rate, CRP)
   e. mRNA for analysis of GATA 2, GATA-3, PPARγ, C/EBPα and SREBP-1
   f. DNA will be collected for HTR 2C and HTR2A
   g. Serum pregnancy test if indicated.
Subjects will fill out the Activity Level Questionnaire.
   a. Waist and hip measurements will be documented.
   b. Waist-to-hip ratio will be calculated.
   c. Body mass index will be assessed.
   d. Study medication will be dispensed and patients will be instructed to take one pill 15 minutes prior to each meal to total 3 pills per day.
   e. Subjects who have a diagnosis of Schizophrenia will have a PANSS and Brief Psychiatric Rating Scale completed (BPRS).

Subjects who have a bipolar diagnosis will complete the Young Mania Rating Scale (YMRS) and Mood Disorders Questionnaire (MDQ).

Visit 3 (6 weeks after visit 2, +/−7 days):
Subject's Informed Consent will again be reviewed.
The diet diary will be collected and a new one dispensed.
Adverse Events or Serious Adverse Events will be collected and assessed. (A physical exam will be conducted if symptoms require further evaluation.)
Drug accountability will be completed. Subjects need to be >80% compliant with medications, or need to be reinstructed on medication use.
Study medication will be dispensed.
Concomitant medications will be reviewed.
The following measurements will be completed:
   a. Vital Signs (blood pressure, heart rate)
   b. Weight
   c. Waist and Hip measurements (hip-to-waist ratio)
   d. EKG, BMI
The following labs will be completed:
   a. mRNA for analysis of GATA 2, GATA-3, PPARγ, C/EBPα and SREBP-1
   b. CMP
   c. CBC
   d. Cortisol
   e. ACTH
   f. Immune assay (SED rate, CRP)
   g. Serum pregnancy test if indicated
Subjects who have a diagnosis of Schizophrenia will have a PANSS and the Brief Psychiatric Rating Scale (BPRS) completed.
Subjects who have a bipolar diagnosis will have the Young Mania Rating Scale (YMRS) completed.

Visit 4 (12 weeks after visit 2, +/−7 days):
Subject's Informed Consent will again be reviewed.
The diet diary will be collected and reviewed.
Drug accountability will be completed. Subjects need to be >80% compliant with medications, or need to be reinstructed on medication use.
Concomitant medications will be reviewed.
Subjects will fill out Activity Level Questionnaire.
A physical exam will be performed.
The following labs will be collected:
   a. CMP
   b. CBC
   c. mRNA for analysis of GATA 2, GATA-3, PPARγ, C/EBPα and SREBP-1
   d. Lipid Panel (total cholesterol, triglycerides, LDL, HDL)
   e. Thyroid (TSH, Free T4)
   f. Immune assay (SED rate, CRP)
   g. Cortisol
   h. ACTH
   i. Serum pregnancy test if indicated
The following measurements will be obtained and recorded:
   a. Vital Signs (blood pressure, heart rate, weight)
   b. EKG
   c. Body Fat measurement by whole body (DXA) scan
   d. Waist and Hip measurements
   e. Waist-to-hip ratio
   f. BMI
Subjects who have a diagnosis of Schizophrenia will have a PANSS and the Brief Psychiatric Rating Scale completed.
Subjects who have a bipolar diagnosis will have the Young Mania Rating Scale (YMRS) completed.

| Visit number | 1 | 2 | 3 | 4 |
|---|---|---|---|---|
| Day | 0 | 7 | 49 | 91 |
| Week | 0 | 1 | 6 | 12 |
| Informed Consent | X | | | |
| Medical History | x | | | |
| Dietary History | x | | | |
| Physical Exam | x | | | x |
| Informed Consent reinforcement | | x | x | x |
| Activity Level Questionnaire | | x | x | x |
| Dispense Diet Diary | x | x | x | |
| Collect Diet Diary | | X | X | x |
| Dispense medication | | x | x | |
| Perform Drug accountability | | | X | X |
| Concomitant Medications | x | x | x | x |
| Whole body DXA for body fat percentage | | x | | x |
| Assess Adverse Events | | | X | X |
| Waist and Hip measurements | | x | x | x |
| Waist to hip ratio | | X | x | x |
| Weight | x | x | x | x |
| Height | x | | | |
| BMI | x | X | x | x |
| Vital Signs (blood pressure, pulse) | x | x | x | x |
| CMP | x | | x | x |
| CBC | X | | X | x |
| Genetic Analysis (GATA-2 and -3, PPARγ, SREBP-1, | | x | x | x |
| C/EBPα) | | | | |
| Lipids (total cholesterol, trigylcerides, LDL, HDL) | x | | | x |
| Thyroid (TSH, FT4) | x | | | x |
| Serum Pregnancy test on all females | X | X* | X* | X* |
| Sex Hormone (estrogen progesterone and testosterone) | | X | | X |
| SED rate | | x | x | x |
| CRP | | x | x | x |
| Cortisol (am), ACTH | | X | x | x |
| EKG | | x | x | x |
| Vitamin D | | X | | x |
| DNA for HTR 2C and HTR 2A | | x | | |
| Positive and Negative Symptoms of Schizophrenia | | X | X | X |
| (PANSS) and Brief Psychiatric Rating Scale (BPRS) if | | | | |
| diagnosed with Schizophrenia | | | | |
| Young Mania Rating Scale (YMRS) if diagnosed with | | x | x | x |
| Bipolar Disorder | | | | |
| Mood Disorders Questionnaire (MDQ) | | x | | |

X* Female subjects who suspect they may be pregnant during the study will have a serum pregnancy test completed.

### Questionnaires:

### Young Mania Rating Scale (YMRS):

This is the most frequently utilized rating scale to assess manic symptoms in bipolar subjects. The scale has 11 items and is based upon the patient's subjective report of his or her clinical condition over the past 48 hours. Each item is given a severity rating. There are 4 items that are rated at twice the weight of the anchor points to grade the severity. Items are graded on a 0 to 4 scale.

### Mood Disorders Questionnaire (MDS):

This is a useful tool for and screening instrument for bipolar spectrum disorder in an outpatient setting. This is a self-report, single-page, inventory, which screens for a lifetime history of a manic or hypomanic syndrome. 13 items are rated on a 4-point scale ranging from “no problem” to “serious problem”.

### Brief Psychiatric Rating Scale (BPRS):

This is a widely used, brief scale that measures major psychotic and nonpyschotic symptoms in individuals with a major psychiatric disorder, particularly schizophrenia. This is an 18-items are rated on a seven point scale (1=not present, and 7=most severe).

### Positive and Negative Symptom Scale for Schizophrenia (PANSS):

This is a 30 item rating scale that is specifically developed to assess individuals with schizophrenia and is used widely in research. This scale is based upon the premise that schizophrenia has two distinct syndromes, a positive and negative syndrome. The positive syndrome includes productive features such as delusions and hallucinations, while the negative syndrome includes those features which are lacking/poorly developed in individuals with schizophrenia, such as social withdrawal and flattened or blunted affect.

Activity Level Questionnaire: This exercise questionnaire will contain the following questions and will be used to assess if subjects have maintained their habits during the study.

### Exercise Questionnaire

| Question | Response | | |
|---|---|---|---|
| Are you currently exercising? | Yes | No | |
| If you are not currently exercising, how long | | | |
| has it been since you have worked out regularly | | | |
| (weeks, months, years)? | | | |
| If you are currently exercising, how long have | | | |
| you been doing your current fitness routine | | | |
| (weeks, months, years)? | | | |
| How many days/week do you currently | | | |
| exercise? | | | |
| How long is each session of activity? | | | |
| What level of intensity is your typical activity | Light | Moderate | Heavy |
| session? | | | |
| What type of activity do you generally do | | | |
| for your activity session? | | | |
| Where do you exercise (home, gym, etc)? | | | |

| Level of | | |
|---|---|---|
| Intensity | RPE | Physical Cues |
| Light | Easy | Does not induce sweating unless it's |
| | | a hot, humid day. There is no |
| | | noticeable change in breathing |
| | | patterns. |
| Moderate | Somewhat | Will break a sweat after performing |
| | hard | the activity for about 10 minutes. |
| | | Breathing becomes deeper and more |
| | | frequent. You can carry on a |
| | | conversation but not sing. |
| Heavy | Hard | Will break a sweat after 3-5 minutes. |
| | | Breathing is deep and rapid. You can |
| | | only talk in short phrases. |

### Diet Log:

Subjects will keep a diet log of everything they eat and drink for seven consecutive days between visits 1-2, 2-3, and visits 3-4. Evaluation of the total number of calories, fat, carbohydrates, and protein will be assessed.

### Laboratory Analysis:

At the screening visit, a total of 10 ml (about 2 teaspoons) of blood will be drawn for the following tests: CMP, CBC, Lipid panel, Thyroid (TSH, Free T4), and beta HCG.

The randomization visit (Visit 2) will include at total of 24 ml (about 2 tablespoons) of blood to be drawn for the following tests: Cortisol, ACTH, estrogen, progesterone, testosterone, Vitamin D, SED rate, CRP, mRNA for genetic analysis of GATA-2 and GATA-3, PPARγ, SREBP-1, C/EBPα, DNA for HTR 2C and HTR 2A, serum pregnancy test if indicated.

Visit 3 labs will include CMP, CBC, mRNA, Cortisol, ACTH, SED rate, CRP, and serum pregnancy test if indicated. The total amount drawn at this visit will include 20 ml (about 1½ tablespoons).

Visit 4 labs will include CMP, CBC, mRNA, Cortisol, ACTH, Lipid Panel, TSH, Free T4, Vitamin D, and immune assay. The total amount of blood drawn will include 12 ml (about 2 teaspoons).

A Comprehensive Metabolic Panel (CMP) will include BUN, Albumin, ALT, AST, Total Bilirubin, Calcium, Carbon Dioxide, Chloride, Creatinine, Glucose, Alkaline Phosphatase, Potassium, Total Protein, and Sodium.

A Lipid Panel will include total cholesterol, triglycerides, LDL, HDL, and LDL/HDL ratio.

An Immune assay will include IL-6, TNFα, SED rate, C-reactive protein.

A Complete Blood Count (CBC) will include RBC, WBC, Hemoglobin, Hematocrit, Platelet Count, RBC Indices (MCV, MCH, MCHC), and Automated 5-part WBC Differential.

Subjects will need to be fasting for the CMP (on visits 1, 3, and 4).

Serum pregnancy tests will be performed on all females prior to randomization. For the duration of the study, in case of suspicion of pregnancy, another serum pregnancy test will be performed.

### Study Medication and Placebo:

The Avera McKennan Hospital Pharmacy will compound IHBG-10 in 250 mg capsules. Subjects will take two capsules 15 minutes prior to any meals. Subjects will receive study medication or placebo at Visit 2 (randomization) and Visit 3. At Visit 3 and Visit 5 (final visit), subjects will return the previously dispensed capsules. Study medication previously dispensed to a subject and returned medication may not be re-dispensed to another subject. Medication compliance will be assessed at Visit 3 and Visit 4.

### Blinding:

Subjects in the study will not be blinded to their treatment. Subjects will be sent letters at the conclusion of the study regarding their final laboratory work. At the principal investigator's discretion, any subject with laboratory measures outside of the range of normal that is in need of follow-up will be contacted by phone or subject's stated preferred method of contact.

### Concomitant Therapies and Non-Drug Therapies:

All concomitant medications taken since informed consent and during the study will be recorded in the source document. Subjects will be encouraged to maintain a steady dose of concomitant medications throughout the study. All changes in concomitant medications or adjustments to dosage will be recorded in the source document.

Subjects will be encouraged to maintain consistency with their diet, exercise habits, alcohol consumption, and smoking habits throughout the study.

### Subject Completion and Withdrawal:

Subjects will be considered to have completed the study when they have attended all visits, and all assessments have been performed. Any subject who enters the study but does not complete the study according to the above definition will be considered a withdrawal, irrespective of whether they have received any study medication.

Some reasons why a subject may withdraw from the study may include, but are not limited to the following situations:
A subject may request withdrawal from the study.
If in the investigator's opinion, continuation in the study would be detrimental to the subject's well being.
The sponsor requests the subject's withdrawal.

When a subject withdraws from the study, all medication and study devices should be returned and all attempts must be made to complete a final visit. Study-related medication that is returned from subjects who have withdrawn from the study may not be re-dispensed.

### Adverse Events and Serious Adverse Events:

### Adverse Event (AE):

An adverse event is defined as follows: Any untoward medical occurrence in the subject temporarily associated with the use of a medicinal product, whether or not considered related to the medicinal product. An AE can therefore be any unfavorable and unintended sign (including an abnormal laboratory finding), symptom or disease (new or exacerbated) temporally associated with the use of a medicinal product. For marketed medicinal products this also includes abuse or misuse of the product. All adverse events will be followed throughout the study.

### Serious Adverse Event (SAE):

A serious adverse event is any untoward medical occurrence that, at any dose:
results in death;
is life-threatening;
requires hospitalization;
results in disability/incapacity; or
is a congenital anomaly/birth defect.

All SAEs must be reported to the Avera Institutional Review Board within ten working days of the event, or upon learning of the event.

### Study Analysis:

Data Collection: Study staff will fill out source documentation with the specific data points as listed in the protocol. After subjects have been seen in the clinic and all data points collected, the study staff will enter the data points on a spreadsheet specifically designed for the data collection. Periodically, the data will be reviewed for accuracy as entered from the source document to the final spreadsheet. The spreadsheet will be used for statistical analysis.

Analysis: Descriptive statistics, such as means, standard deviations and statistical distributions, will be explored for the weight, BMI, and lab values. Further comparisons will be completed as determined during the analysis of lab and other outcomes. Statistical analysis of variance and covariance will be use to compare these outcome measures between intervention and control groups and to test whether there is a significant difference. Other advanced statistical models will be employed depending on the statistical distributions observed.

### Ethical Conduct of the Study:

This study will be conducted in accordance with “good clinical practice” (GCP) and all applicable regulatory requirements including, where applicable, the October 1996 version of the Declaration of Helsinki

All applicable regulatory documents will be reviewed by the Avera Institutional Review Board and will have received approval prior to any implementation of the protocol procedures.

Informed consent will be obtained before the subject can participate in the study. The contents and process of obtaining informed consent will be in accordance with all applicable regulatory requirements.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim herein:

1. A method for treating or preventing obesity in a subject in need thereof, comprising:
administering to said subject a therapeutically effective amount of berberine, berberine salt, berberine analog or combination thereof, wherein the subject consumes a high fat diet or wherein said subject has be exposed to a pharmacologic agent which induces adipogenesis as a side effect.

2. The method of claim 1, wherein said administering suppresses the appetite of said subject.

3. The method of claim 1, wherein the berberine salt is selected from the group consisting of berberine chloride, berberine phosphate, berberine sulphate, berberine bi-sulphate, berberine tannate, berberine hemisulphate, and berberine citrate.

4. The method of claim 1, wherein the berberine analog is an alkaloid extracted from Sanguinarine, Coptisine, or Goldenseal.

5. The method of claim 1, wherein the pharmacologic agent selected from the group consisting of a sedative, an antipsychotic, a tranquilizer, an antidepressant, and an anticonvulsant.

6. The method of claim 5, wherein the pharmacologic agent is an antipsychotic.

7. The method of claim 6, wherein the antipsychotic does not induce expression of fatty acid synthase (FAS).

8. The method of claim 6, wherein the antipsychotic is clozapine or risperidone.

9. The method of claim 1, further comprising a natural product.

10. The method of claim 9, wherein said natural product is amentoflavone.

11. A composition comprising berberine or a berberine analog and a pharmacologic agent selected from the group consisting of an antipsychotic, a tranquilizer, an antidepressant, and an anticonvulsant.

12. The composition of claim 11, wherein the pharmacologic agent is an antipsychotic.

13. The composition of claim 12, wherein the pharmacologic agent is clozapine or risperidone.

14. The composition of claim 13, wherein the pharmacologic agent does not induce fatty acid synthase expression.

15. The composition of claim 11, further comprising a natural product.

16. The composition of claim 15, wherein the natural product is amentoflavone.

17. A method for treating or preventing obesity in a subject comprising:
administering a therapeutically effective amount of a combination of berberine, berberine salt, or berberine analog and a natural product to a subject in need thereof, wherein said subject has been exposed to a pharmacologic agent which induces adipogenesis as a side effect.

18. The method of claim 17, wherein said combination enhances the inhibition of adipogenesis in said subject such that the amount of berberine, berberine salt, or berberine analog administered which is necessary to achieve said inhibition is reduced relative to administration of berberine, berberine salt, or berberine analog alone.

19. The method of claim 17, wherein said berberine salt is selected from the group consisting of berberine chloride, berberine phosphate, berberine sulphate, berberine bi-sulphate, berberine tannate, berberine hemisulphate and berberine citrate.

20. The method of claim 19, wherein said berberine analog is an alkaloid extracted from Sanguinarine, Coptisine, or Goldenseal.

21. The method of claim 17, wherein the pharmacologic agent is selected from the group consisting of a sedative, an antipsychotic, a tranquilizer, an antidepressant, and an anticonvulsant.

22. The method of claim 21, wherein the pharmacologic agent is an antipsychotic.

23. The method of claim 22, wherein the antipsychotic does not induce expression of fatty acid synthase (FAS).

24. The method of claim 22, wherein said antipsychotic is clozapine or risperidone.

25. The method of claim 17, wherein the natural product is amentoflavone.

26. The use of a composition comprising berberine or a berberine analog and a pharmacologic agent selected from the group consisting of an antipsychotic, a tranquilizer, an antidepressant, and an anticonvulsant in the manufacture of a medicament for the treatment of obesity in a subject in need thereof.

27. The use of claim 26, wherein the composition further comprises amentoflavone.

28. The use of claim 26, wherein the pharmacologic agent is an antipsychotic.

29. The use of claim 28, wherein the antipsychotic does not induce expression of fatty acid synthase (FAS).

30. The use of claim 26, wherein said antipsychotic is clozapine or risperidone.
